SEQUENCE LISTING (293 sequences)

SEQ ID NO: 1 - protein, 5 aa, synthetic construct
  REGION 1..5: note = human mAb CDR:HC1; clone: p2.3
DSYGM

SEQ ID NO: 2 - protein, 5 aa, synthetic construct
  REGION 1..5: note = human mAb CDR:HC1; clone: p2.8
DSYGM

SEQ ID NO: 3 - protein, 5 aa, synthetic construct
  REGION 1..5: note = human mAb CDR:HC1; clone: p2.9
SVYGM

SEQ ID NO: 4 - protein, 5 aa, synthetic construct
  REGION 1..5: note = human mAb CDR:HC1; clone: p2.10
SVYAM

SEQ ID NO: 5 - protein, 5 aa, synthetic construct
  REGION 1..5: note = human mAb CDR:HC1; clone: p2.4
SNYVM

SEQ ID NO: 6 - protein, 5 aa, synthetic construct
  REGION 1..5: note = human mAb CDR:HC1; clone: p2.11
GVYGM

SEQ ID NO: 7 - protein, 5 aa, synthetic construct
  REGION 1..5: note = human mAb CDR:HC1; clone: p2.12
SIYAM

SEQ ID NO: 8 - protein, 5 aa, synthetic construct
  REGION 1..5: note = human mAb CDR:HC1; clone: p2.13
SVYGM

SEQ ID NO: 9 - protein, 5 aa, synthetic construct
  REGION 1..5: note = human mAb CDR:HC1; clone: p2.14
DIYGM

SEQ ID NO: 10 - protein, 5 aa, synthetic construct
  REGION 1..5: note = human mAb CDR:HC1; clone: p2.15
DIYGM

SEQ ID NO: 11 - protein, 5 aa, synthetic construct
  REGION 1..5: note = human mAb CDR:HC1; clone: p2.16
SNYVM

SEQ ID NO: 12 - protein, 5 aa, synthetic construct
  REGION 1..5: note = human mAb CDR:HC1; clone: p2.17
NVYAM

SEQ ID NO: 13 - protein, 5 aa, synthetic construct
  REGION 1..5: note = human mAb CDR:HC1; clone: p2.18
DIYGM

SEQ ID NO: 14 - protein, 5 aa, synthetic construct
  REGION 1..5: note = human mAb CDR:HC1; clone: p2.19
SIYAM

SEQ ID NO: 15 - protein, 5 aa, synthetic construct
  REGION 1..5: note = human mAb CDR:HC1; clone: p2.20
DIYGM

SEQ ID NO: 16 - protein, 5 aa, synthetic construct
  REGION 1..5: note = human mAb CDR:HC1; clone: p2.21
SVYGM

SEQ ID NO: 17 - protein, 5 aa, synthetic construct
  REGION 1..5: note = human mAb CDR:HC1; clone: p1.22
INFAM

SEQ ID NO: 18 - protein, 5 aa, synthetic construct
  REGION 1..5: note = human mAb CDR:HC1; clone: p1.23
SSYAM

SEQ ID NO: 19 - protein, 5 aa, synthetic construct
  REGION 1..5: note = human mAb CDR:HC1; clone: p1.24
SSYAM

SEQ ID NO: 20 - protein, 5 aa, synthetic construct
  REGION 1..5: note = human mAb CDR:HC1; clone: p1.25
RNYGM

SEQ ID NO: 21 - protein, 5 aa, synthetic construct
  REGION 1..5: note = human mAb CDR:HC1; clone: p1.1
SSYAM

SEQ ID NO: 22 - protein, 5 aa, synthetic construct
  REGION 1..5: note = human mAb CDR:HC1; clone: p1.26
SSYAM

SEQ ID NO: 23 - protein, 5 aa, synthetic construct
  REGION 1..5: note = human mAb CDR:HC1; clone: p1.27
DVYAM

SEQ ID NO: 24 - protein, 5 aa, synthetic construct
  REGION 1..5: note = human mAb CDR:HC1; clone: p1.28
SSYAM

SEQ ID NO: 25 - protein, 5 aa, synthetic construct
  REGION 1..5: note = human mAb CDR:HC1; clone: p1.29
SSYAM

SEQ ID NO: 26 - protein, 5 aa, synthetic construct
  REGION 1..5: note = human mAb CDR:HC1; clone: p1.30
DVYAM

SEQ ID NO: 27 - protein, 5 aa, synthetic construct
  REGION 1..5: note = human mAb CDR:HC1; clone: p1.6
DVYAM

SEQ ID NO: 28 - protein, 5 aa, synthetic construct
  REGION 1..5: note = human mAb CDR:HC1; clone: p1.2
INFAM

SEQ ID NO: 29 - protein, 5 aa, synthetic construct
  REGION 1..5: note = human mAb CDR:HC1; clone: p1.31
INFAM

SEQ ID NO: 30 - protein, 5 aa, synthetic construct
  REGION 1..5: note = human mAb CDR:HC1; clone: p1.5
DVYAM

SEQ ID NO: 31 - protein, 5 aa, synthetic construct
  REGION 1..5: note = human mAb CDR:HC1; clone: p1.32
DVYAM

SEQ ID NO: 32 - protein, 5 aa, synthetic construct
  REGION 1..5: note = human mAb CDR:HC1; clone: p1.33
RNYGM

SEQ ID NO: 33 - protein, 5 aa, synthetic construct
  REGION 1..5: note = human mAb CDR:HC1; clone: p1.39
INFAM

SEQ ID NO: 34 - protein, 5 aa, synthetic construct
  REGION 1..5: note = human mAb CDR:HC1; clone: p1.22
INFAM

SEQ ID NO: 35 - protein, 5 aa, synthetic construct
  REGION 1..5: note = human mAb CDR:HC1; clone: p3.1.34
SNYAM

SEQ ID NO: 36 - protein, 5 aa, synthetic construct
  REGION 1..5: note = human mAb CDR:HC1; clone: p3.1.35
SSYGM

SEQ ID NO: 37 - protein, 5 aa, synthetic construct
  REGION 1..5: note = human mAb CDR:HC1; clone: p3.1.36
SYYAM

SEQ ID NO: 38 - protein, 5 aa, synthetic construct
  REGION 1..5: note = human mAb CDR:HC1; clone: p3.1.37
RSYAM

SEQ ID NO: 39 - protein, 5 aa, synthetic construct
  REGION 1..5: note = human mAb CDR:HC1; clone: p3.2.38
SSYGM

SEQ ID NO: 40 - protein, 5 aa, synthetic construct
  REGION 1..5: note = human mAb CDR:HC1; clone: p3.2.40
SYYAM

SEQ ID NO: 41 - protein, 15 aa, synthetic construct
  REGION 1..15: note = human mAb CDR:HC2; clone: p2.3
SINVGDNTYYADSVR

SEQ ID NO: 42 - protein, 15 aa, synthetic construct
  REGION 1..15: note = human mAb CDR:HC2; clone: p2.8
SINVGDNTYYADSVR

SEQ ID NO: 43 - protein, 16 aa, synthetic construct
  REGION 1..16: note = human mAb CDR:HC2; clone: p2.9
RISGSGDSTFYADSVK

SEQ ID NO: 44 - protein, 16 aa, synthetic construct
  REGION 1..16: note = human mAb CDR:HC2; clone: p2.10
RISGNGGSTYYADSVK

SEQ ID NO: 45 - protein, 16 aa, synthetic construct
  REGION 1..16: note = human mAb CDR:HC2; clone: p2.4
AISHSGGTTYYADSVE

SEQ ID NO: 46 - protein, 16 aa, synthetic construct
  REGION 1..16: note = human mAb CDR:HC2; clone: p2.11
RISGSGDSTFYADSVK

SEQ ID NO: 47 - protein, 16 aa, synthetic construct
  REGION 1..16: note = human mAb CDR:HC2; clone: p2.12
GISGSGALTYYADSVK

SEQ ID NO: 48 - protein, 16 aa, synthetic construct
  REGION 1..16: note = human mAb CDR:HC2; clone: p2.13
RISGSGDSTFYADSVK

SEQ ID NO: 49 - protein, 16 aa, synthetic construct
  REGION 1..16: note = human mAb CDR:HC2; clone: p2.14
RISVRGRGAEYADSVK

SEQ ID NO: 50 - protein, 16 aa, synthetic construct
  REGION 1..16: note = human mAb CDR:HC2; clone: p2.15
RISGNGGSTFYSESVK

SEQ ID NO: 51 - protein, 16 aa, synthetic construct
  REGION 1..16: note = human mAb CDR:HC2; clone: p2.16
AISHSGGTTYYADSVK

SEQ ID NO: 52 - protein, 16 aa, synthetic construct
  REGION 1..16: note = human mAb CDR:HC2; clone: p2.17
RISGNGGSTYYAESVK

SEQ ID NO: 53 - protein, 16 aa, synthetic construct
  REGION 1..16: note = human mAb CDR:HC2; clone: p2.18
RISGSGGTTFYAESVK

SEQ ID NO: 54 - protein, 16 aa, synthetic construct
  REGION 1..16: note = human mAb CDR:HC2; clone: p2.19
GISGSGALTYYADSVK

SEQ ID NO: 55 - protein, 16 aa, synthetic construct
  REGION 1..16: note = human mAb CDR:HC2; clone: p2.20
RISVRGRGAEYADSVK

SEQ ID NO: 56 - protein, 16 aa, synthetic construct
  REGION 1..16: note = human mAb CDR:HC2; clone: p2.21
RISGSGDSTFYADSVK

SEQ ID NO: 57 - protein, 18 aa, synthetic construct
  REGION 1..18: note = human mAb CDR:HC2; clone: p1.22
LISDDGRGNRPYYADSVK

SEQ ID NO: 58 - protein, 14 aa, synthetic construct
  REGION 1..14: note = human mAb CDR:HC2; clone: p1.23
RISDYTIYYADSVK

SEQ ID NO: 59 - protein, 14 aa, synthetic construct
  REGION 1..14: note = human mAb CDR:HC2; clone: p1.24
RISDYTIYYADSVK

SEQ ID NO: 60 - protein, 16 aa, synthetic construct
  REGION 1..16: note = human mAb CDR:HC2; clone: p1.25
VISGSGGSTYYIDSVK

SEQ ID NO: 61 - protein, 14 aa, synthetic construct
  REGION 1..14: note = human mAb CDR:HC2; clone: p1.1
RISDYTIYYADSVK

SEQ ID NO: 62 - protein, 14 aa, synthetic construct
  REGION 1..14: note = human mAb CDR:HC2; clone: p1.26
RISDYTIYYADSVK

SEQ ID NO: 63 - protein, 16 aa, synthetic construct
  REGION 1..16: note = human mAb CDR:HC2; clone: p1.27
LISGTGRMTYYADSVK

SEQ ID NO: 64 - protein, 14 aa, synthetic construct
  REGION 1..14: note = human mAb CDR:HC2; clone: p1.28
RISDYTIYYADSVK

SEQ ID NO: 65 - protein, 14 aa, synthetic construct
  REGION 1..14: note = human mAb CDR:HC2; clone: p1.29
RISDYTIYYADSVK

SEQ ID NO: 66 - protein, 16 aa, synthetic construct
  REGION 1..16: note = human mAb CDR:HC2; clone: p1.30
LISGTGRMTYYADSVK

SEQ ID NO: 67 - protein, 16 aa, synthetic construct
  REGION 1..16: note = human mAb CDR:HC2; clone: p1.6
LISGTGRMTYYADSVK

SEQ ID NO: 68 - protein, 18 aa, synthetic construct
  REGION 1..18: note = human mAb CDR:HC2; clone: p1.2
LISDDGRGNRPYYADSVK

SEQ ID NO: 69 - protein, 18 aa, synthetic construct
  REGION 1..18: note = human mAb CDR:HC2; clone: p1.31
LISDDGRGNRPYYADSVK

SEQ ID NO: 70 - protein, 16 aa, synthetic construct
  REGION 1..16: note = human mAb CDR:HC2; clone: p1.5
LISGTGRMTYYADSVK

SEQ ID NO: 71 - protein, 16 aa, synthetic construct
  REGION 1..16: note = human mAb CDR:HC2; clone: p1.32
LISGTGRMTYYADSVK

SEQ ID NO: 72 - protein, 16 aa, synthetic construct
  REGION 1..16: note = human mAb CDR:HC2; clone: p1.33
VISGSGGSTYYIDSVK

SEQ ID NO: 73 - protein, 18 aa, synthetic construct
  REGION 1..18: note = human mAb CDR:HC2; clone: p1.39
LISDDGRGNRPYYADSVK

SEQ ID NO: 74 - protein, 18 aa, synthetic construct
  REGION 1..18: note = human mAb CDR:HC2; clone: p1.22
LISDDGRGNRPYYADSVK

SEQ ID NO: 75 - protein, 21 aa, synthetic construct
  REGION 1..21: note = human mAb CDR:HC2; clone: p3.1.34
SISGNGISHSGGSTYYADSVK

SEQ ID NO: 76 - protein, 16 aa, synthetic construct
  REGION 1..16: note = human mAb CDR:HC2; clone: p3.1.35
IISGDGVITSYADSVK

SEQ ID NO: 77 - protein, 16 aa, synthetic construct
  REGION 1..16: note = human mAb CDR:HC2; clone: p3.1.36
RISGSGGSTFYADSVK

SEQ ID NO: 78 - protein, 21 aa, synthetic construct
  REGION 1..21: note = human mAb CDR:HC2; clone: p3.1.37
SISGNGISHSGGSTYYADSVK

SEQ ID NO: 79 - protein, 16 aa, synthetic construct
  REGION 1..16: note = human mAb CDR:HC2; clone: p3.2.38
IISGDGVITSYADSVK

SEQ ID NO: 80 - protein, 16 aa, synthetic construct
  REGION 1..16: note = human mAb CDR:HC2; clone: p3.2.40
RISGSGGSTFYADSVK

SEQ ID NO: 81 - protein, 8 aa, synthetic construct
  REGION 1..8: note = human mAb CDR:HC3; clone: p2.3
WNSFFDYW

SEQ ID NO: 82 - protein, 8 aa, synthetic construct
  REGION 1..8: note = human mAb CDR:HC3; clone: p2.8
WNSFFDYW

SEQ ID NO: 83 - protein, 9 aa, synthetic construct
  REGION 1..9: note = human mAb CDR:HC3; clone: p2.9
TWNNFFDYW

SEQ ID NO: 84 - protein, 9 aa, synthetic construct
  REGION 1..9: note = human mAb CDR:HC3; clone: p2.10
TLNNFFDYW

SEQ ID NO: 85 - protein, 12 aa, synthetic construct
  REGION 1..12: note = human mAb CDR:HC3; clone: p2.4
DLGYWNNFFDYW

SEQ ID NO: 86 - protein, 9 aa, synthetic construct
  REGION 1..9: note = human mAb CDR:HC3; clone: p2.11
TWNNFFDYW

SEQ ID NO: 87 - protein, 9 aa, synthetic construct
  REGION 1..9: note = human mAb CDR:HC3; clone: p2.12
PLNNFFDYW

SEQ ID NO: 88 - protein, 9 aa, synthetic construct
  REGION 1..9: note = human mAb CDR:HC3; clone: p2.13
TWNNFFDYW

SEQ ID NO: 89 - protein, 11 aa, synthetic construct
  REGION 1..11: note = human mAb CDR:HC3; clone: p2.14
ENNWNSFFDYW

SEQ ID NO: 90 - protein, 9 aa, synthetic construct
  REGION 1..9: note = human mAb CDR:HC3; clone: p2.15
TLNNFFDYW

SEQ ID NO: 91 - protein, 12 aa, synthetic construct
  REGION 1..12: note = human mAb CDR:HC3; clone: p2.16
DLGYWNNFFDYW

SEQ ID NO: 92 - protein, 9 aa, synthetic construct
  REGION 1..9: note = human mAb CDR:HC3; clone: p2.17
TWNNFFDYW

SEQ ID NO: 93 - protein, 9 aa, synthetic construct
  REGION 1..9: note = human mAb CDR:HC3; clone: p2.18
TLNNFFDYW

SEQ ID NO: 94 - protein, 9 aa, synthetic construct
  REGION 1..9: note = human mAb CDR:HC3; clone: p2.19
PLNNFFDYW

SEQ ID NO: 95 - protein, 11 aa, synthetic construct
  REGION 1..11: note = human mAb CDR:HC3; clone: p2.20
ENNWNSFFDYW

SEQ ID NO: 96 - protein, 9 aa, synthetic construct
  REGION 1..9: note = human mAb CDR:HC3; clone: p2.21
TWNNFFDYW

SEQ ID NO: 97 - protein, 10 aa, synthetic construct
  REGION 1..10: note = human mAb CDR:HC3; clone: p1.22
VSWDVFFDYW

SEQ ID NO: 98 - protein, 16 aa, synthetic construct
  REGION 1..16: note = human mAb CDR:HC3; clone: p1.23
SGPKSGKYWNNFFDSW

SEQ ID NO: 99 - protein, 16 aa, synthetic construct
  REGION 1..16: note = human mAb CDR:HC3; clone: p1.24
SGPKSGKYWNNFFDSW

SEQ ID NO: 100 - protein, 12 aa, synthetic construct
  REGION 1..12: note = human mAb CDR:HC3; clone: p1.25
DRGNSYGFYDYW

SEQ ID NO: 101 - protein, 16 aa, synthetic construct
  REGION 1..16: note = human mAb CDR:HC3; clone: p1.1
SGPKSGKYWNNFFDSW

SEQ ID NO: 102 - protein, 16 aa, synthetic construct
  REGION 1..16: note = human mAb CDR:HC3; clone: p1.26
SGPKSGKYWNNFFDSW

SEQ ID NO: 103 - protein, 11 aa, synthetic construct
  REGION 1..11: note = human mAb CDR:HC3; clone: p1.27
DPFGHGFFDYW

SEQ ID NO: 104 - protein, 16 aa, synthetic construct
  REGION 1..16: note = human mAb CDR:HC3; clone: p1.28
SGPKSGKYWNNFFDSW

SEQ ID NO: 105 - protein, 16 aa, synthetic construct
  REGION 1..16: note = human mAb CDR:HC3; clone: p1.29
SGPKSGKYWNNFFDSW

SEQ ID NO: 106 - protein, 11 aa, synthetic construct
  REGION 1..11: note = human mAb CDR:HC3; clone: p1.30
DPFGHGFFDYW

SEQ ID NO: 107 - protein, 11 aa, synthetic construct
  REGION 1..11: note = human mAb CDR:HC3; clone: p1.6
DPFGHGFFDYW

SEQ ID NO: 108 - protein, 10 aa, synthetic construct
  REGION 1..10: note = human mAb CDR:HC3; clone: p1.2
VSWDVFFDYW

SEQ ID NO: 109 - protein, 10 aa, synthetic construct
  REGION 1..10: note = human mAb CDR:HC3; clone: p1.31
VSWDVFFDYW

SEQ ID NO: 110 - protein, 11 aa, synthetic construct
  REGION 1..11: note = human mAb CDR:HC3; clone: p1.5
DPFGHGFFDYW

SEQ ID NO: 111 - protein, 11 aa, synthetic construct
  REGION 1..11: note = human mAb CDR:HC3; clone: p1.32
DPFGHGFFDYW

SEQ ID NO: 112 - protein, 12 aa, synthetic construct
  REGION 1..12: note = human mAb CDR:HC3; clone: p1.33
DRGNSYGFYDYW

SEQ ID NO: 113 - protein, 10 aa, synthetic construct
  REGION 1..10: note = human mAb CDR:HC3; clone: p1.39
VSWDVFFDYW

SEQ ID NO: 114 - protein, 10 aa, synthetic construct
  REGION 1..10: note = human mAb CDR:HC3; clone: p1.22
VSWDVFFDYW

SEQ ID NO: 115 - protein, 13 aa, synthetic construct
  REGION 1..13: note = human mAb CDR:HC3; clone: p3.1.34
DLGTWNSYGFDYW

SEQ ID NO: 116 - protein, 11 aa, synthetic construct
  REGION 1..11: note = human mAb CDR:HC3; clone: p3.1.35
RDHRSTFFDYW

SEQ ID NO: 117 - protein, 9 aa, synthetic construct
  REGION 1..9: note = human mAb CDR:HC3; clone: p3.1.36
TLNNFFDYW

SEQ ID NO: 118 - protein, 13 aa, synthetic construct
  REGION 1..13: note = human mAb CDR:HC3; clone: p3.1.37
DLGTWNSYGFDYW

SEQ ID NO: 119 - protein, 11 aa, synthetic construct
  REGION 1..11: note = human mAb CDR:HC3; clone: p3.2.38
RDHRSTFFDYW

SEQ ID NO: 120 - protein, 9 aa, synthetic construct
  REGION 1..9: note = human mAb CDR:HC3; clone: p3.2.40
TLNNFFDYW

SEQ ID NO: 121 - protein, 9 aa, synthetic construct
  REGION 1..9: note = human mAb CDR:LC1; clone: p2.3
SQNVRSDLA

SEQ ID NO: 122 - protein, 9 aa, synthetic construct
  REGION 1..9: note = human mAb CDR:LC1; clone: p2.8
SQSVNSKLA

SEQ ID NO: 123 - protein, 9 aa, synthetic construct
  REGION 1..9: note = human mAb CDR:LC1; clone: p2.9
SQTVKTYLA

SEQ ID NO: 124 - protein, 9 aa, synthetic construct
  REGION 1..9: note = human mAb CDR:LC1; clone: p2.10
SQGIVGNLA

SEQ ID NO: 125 - protein, 9 aa, synthetic construct
  REGION 1..9: note = human mAb CDR:LC1; clone: p2.4
SQNVRSDLA

SEQ ID NO: 126 - protein, 9 aa, synthetic construct
  REGION 1..9: note = human mAb CDR:LC1; clone: p2.11
SQTVKTYLA

SEQ ID NO: 127 - protein, 9 aa, synthetic construct
  REGION 1..9: note = human mAb CDR:LC1; clone: p2.12
SQSVNSNLA

SEQ ID NO: 128 - protein, 9 aa, synthetic construct
  REGION 1..9: note = human mAb CDR:LC1; clone: p2.13
SQTVKTYLA

SEQ ID NO: 129 - protein, 9 aa, synthetic construct
  REGION 1..9: note = human mAb CDR:LC1; clone: p2.14
SQSVSSYLA

SEQ ID NO: 130 - protein, 9 aa, synthetic construct
  REGION 1..9: note = human mAb CDR:LC1; clone: p2.15
SQTVGSKLA

SEQ ID NO: 131 - protein, 9 aa, synthetic construct
  REGION 1..9: note = human mAb CDR:LC1; clone: p2.16
SQSVHSYLA

SEQ ID NO: 132 - protein, 9 aa, synthetic construct
  REGION 1..9: note = human mAb CDR:LC1; clone: p2.17
SQSVKTYLA

SEQ ID NO: 133 - protein, 10 aa, synthetic construct
  REGION 1..10: note = human mAb CDR:LC1; clone: p2.18
SHSVSSTYLA

SEQ ID NO: 134 - protein, 9 aa, synthetic construct
  REGION 1..9: note = human mAb CDR:LC1; clone: p2.19
SQNVRSDLA

SEQ ID NO: 135 - protein, 9 aa, synthetic construct
  REGION 1..9: note = human mAb CDR:LC1; clone: p2.20
SQSVGSNLA

SEQ ID NO: 136 - protein, 9 aa, synthetic construct
  REGION 1..9: note = human mAb CDR:LC1; clone: p2.21
SQTVKTYLA

SEQ ID NO: 137 - protein, 9 aa, synthetic construct
  REGION 1..9: note = human mAb CDR:LC1; clone: p1.22
SQPIDVYLA

SEQ ID NO: 138 - protein, 9 aa, synthetic construct
  REGION 1..9: note = human mAb CDR:LC1; clone: p1.23
SQTVGSKLA

SEQ ID NO: 139 - protein, 10 aa, synthetic construct
  REGION 1..10: note = human mAb CDR:LC1; clone: p1.24
SQSVGSYYLA

SEQ ID NO: 140 - protein, 10 aa, synthetic construct
  REGION 1..10: note = human mAb CDR:LC1; clone: p1.25
SQSVSASQLA

SEQ ID NO: 141 - protein, 9 aa, synthetic construct
  REGION 1..9: note = human mAb CDR:LC1; clone: p1.1
SQSVKTDLA

SEQ ID NO: 142 - protein, 9 aa, synthetic construct
  REGION 1..9: note = human mAb CDR:LC1; clone: p1.26
SQSVKTDLA

SEQ ID NO: 143 - protein, 9 aa, synthetic construct
  REGION 1..9: note = human mAb CDR:LC1; clone: p1.27
SQSVNRNLA

SEQ ID NO: 144 - protein, 9 aa, synthetic construct
  REGION 1..9: note = human mAb CDR:LC1; clone: p1.28
SQPINTYLA

SEQ ID NO: 145 - protein, 9 aa, synthetic construct
  REGION 1..9: note = human mAb CDR:LC1; clone: p1.29
SQTVGSKLA

SEQ ID NO: 146 - protein, 9 aa, synthetic construct
  REGION 1..9: note = human mAb CDR:LC1; clone: p1.30
SQSVNRNLA

SEQ ID NO: 147 - protein, 9 aa, synthetic construct
  REGION 1..9: note = human mAb CDR:LC1; clone: p1.6
SQSVNRNLA

SEQ ID NO: 148 - protein, 9 aa, synthetic construct
  REGION 1..9: note = human mAb CDR:LC1; clone: p1.2
SQPIDVYLA

SEQ ID NO: 149 - protein, 9 aa, synthetic construct
  REGION 1..9: note = human mAb CDR:LC1; clone: p1.31
SQPIDVYLA

SEQ ID NO: 150 - protein, 9 aa, synthetic construct
  REGION 1..9: note = human mAb CDR:LC1; clone: p1.5
SQTVGSKLA

SEQ ID NO: 151 - protein, 9 aa, synthetic construct
  REGION 1..9: note = human mAb CDR:LC1; clone: p1.32
SQSVNHNLA

SEQ ID NO: 152 - protein, 9 aa, synthetic construct
  REGION 1..9: note = human mAb CDR:LC1; clone: p1.33
NRSILTSLA

SEQ ID NO: 153 - protein, 9 aa, synthetic construct
  REGION 1..9: note = human mAb CDR:LC1; clone: p1.39
SQPIDVYLA

SEQ ID NO: 154 - protein, 9 aa, synthetic construct
  REGION 1..9: note = human mAb CDR:LC1; clone: p1.22
SQPIDVYLA

SEQ ID NO: 155 - protein, 9 aa, synthetic construct
  REGION 1..9: note = human mAb CDR:LC1; clone: p3.1.34
SQTVNTKLA

SEQ ID NO: 156 - protein, 9 aa, synthetic construct
  REGION 1..9: note = human mAb CDR:LC1; clone: p3.1.35
SQSVSSNLA

SEQ ID NO: 157 - protein, 10 aa, synthetic construct
  REGION 1..10: note = human mAb CDR:LC1; clone: p3.1.36
SQSVSDTYLA

SEQ ID NO: 158 - protein, 9 aa, synthetic construct
  REGION 1..9: note = human mAb CDR:LC1; clone: p3.1.37
SQTVTTKLA

SEQ ID NO: 159 - protein, 9 aa, synthetic construct
  REGION 1..9: note = human mAb CDR:LC1; clone: p3.2.38
SQSVSSNLA

SEQ ID NO: 160 - protein, 10 aa, synthetic construct
  REGION 1..10: note = human mAb CDR:LC1; clone: p3.2.40
SQSVSDTYLA

SEQ ID NO: 161 - protein, 7 aa, synthetic construct
  REGION 1..7: note = human mAb CDR:LC2; clone: p2.3
DASNRAT

SEQ ID NO: 162 - protein, 7 aa, synthetic construct
  REGION 1..7: note = human mAb CDR:LC2; clone: p2.8
DASSRAT

SEQ ID NO: 163 - protein, 7 aa, synthetic construct
  REGION 1..7: note = human mAb CDR:LC2; clone: p2.9
AASSRAA

SEQ ID NO: 164 - protein, 7 aa, synthetic construct
  REGION 1..7: note = human mAb CDR:LC2; clone: p2.10
DASSRAT

SEQ ID NO: 165 - protein, 7 aa, synthetic construct
  REGION 1..7: note = human mAb CDR:LC2; clone: p2.4
GASTRAT

SEQ ID NO: 166 - protein, 7 aa, synthetic construct
  REGION 1..7: note = human mAb CDR:LC2; clone: p2.11
DASTRAT

SEQ ID NO: 167 - protein, 7 aa, synthetic construct
  REGION 1..7: note = human mAb CDR:LC2; clone: p2.12
DASSRAT

SEQ ID NO: 168 - protein, 7 aa, synthetic construct
  REGION 1..7: note = human mAb CDR:LC2; clone: p2.13
DASTRAT

SEQ ID NO: 169 - protein, 7 aa, synthetic construct
  REGION 1..7: note = human mAb CDR:LC2; clone: p2.14
GASTRAT

SEQ ID NO: 170 - protein, 7 aa, synthetic construct
  REGION 1..7: note = human mAb CDR:LC2; clone: p2.15
DASTRAT

SEQ ID NO: 171 - protein, 7 aa, synthetic construct
  REGION 1..7: note = human mAb CDR:LC2; clone: p2.16
DASSRAT

SEQ ID NO: 172 - protein, 7 aa, synthetic construct
  REGION 1..7: note = human mAb CDR:LC2; clone: p2.17
DASDRAT

SEQ ID NO: 173 - protein, 7 aa, synthetic construct
  REGION 1..7: note = human mAb CDR:LC2; clone: p2.18
DASTRAT

SEQ ID NO: 174 - protein, 7 aa, synthetic construct
  REGION 1..7: note = human mAb CDR:LC2; clone: p2.19
GASSRAT

SEQ ID NO: 175 - protein, 7 aa, synthetic construct
  REGION 1..7: note = human mAb CDR:LC2; clone: p2.20
AASSRAT

SEQ ID NO: 176 - protein, 7 aa, synthetic construct
  REGION 1..7: note = human mAb CDR:LC2; clone: p2.21
DASTKAT

SEQ ID NO: 177 - protein, 7 aa, synthetic construct
  REGION 1..7: note = human mAb CDR:LC2; clone: p1.22
GASRRAT

SEQ ID NO: 178 - protein, 7 aa, synthetic construct
  REGION 1..7: note = human mAb CDR:LC2; clone: p1.23
AASSRDT

SEQ ID NO: 179 - protein, 7 aa, synthetic construct
  REGION 1..7: note = human mAb CDR:LC2; clone: p1.24
GASRRAT

SEQ ID NO: 180 - protein, 7 aa, synthetic construct
  REGION 1..7: note = human mAb CDR:LC2; clone: p1.25
DATTRAT

SEQ ID NO: 181 - protein, 7 aa, synthetic construct
  REGION 1..7: note = human mAb CDR:LC2; clone: p1.1
GATTRAT

SEQ ID NO: 182 - protein, 7 aa, synthetic construct
  REGION 1..7: note = human mAb CDR:LC2; clone: p1.26
GATTRAT

SEQ ID NO: 183 - protein, 7 aa, synthetic construct
  REGION 1..7: note = human mAb CDR:LC2; clone: p1.27
DARTRAT

SEQ ID NO: 184 - protein, 7 aa, synthetic construct
  REGION 1..7: note = human mAb CDR:LC2; clone: p1.28
DASTRAT

SEQ ID NO: 185 - protein, 7 aa, synthetic construct
  REGION 1..7: note = human mAb CDR:LC2; clone: p1.29
SASSRAT

SEQ ID NO: 186 - protein, 7 aa, synthetic construct
  REGION 1..7: note = human mAb CDR:LC2; clone: p1.30
DARTRAT

SEQ ID NO: 187 - protein, 7 aa, synthetic construct
  REGION 1..7: note = human mAb CDR:LC2; clone: p1.6
DARTRAT

SEQ ID NO: 188 - protein, 7 aa, synthetic construct
  REGION 1..7: note = human mAb CDR:LC2; clone: p1.2
GANRRAI

SEQ ID NO: 189 - protein, 7 aa, synthetic construct
  REGION 1..7: note = human mAb CDR:LC2; clone: p1.31
GANRRAI

SEQ ID NO: 190 - protein, 7 aa, synthetic construct
  REGION 1..7: note = human mAb CDR:LC2; clone: p1.5
DASTRAT

SEQ ID NO: 191 - protein, 7 aa, synthetic construct
  REGION 1..7: note = human mAb CDR:LC2; clone: p1.32
DARTRAT

SEQ ID NO: 192 - protein, 7 aa, synthetic construct
  REGION 1..7: note = human mAb CDR:LC2; clone: p1.33
DASTRAT

SEQ ID NO: 193 - protein, 7 aa, synthetic construct
  REGION 1..7: note = human mAb CDR:LC2; clone: p1.39
GANRRAI

SEQ ID NO: 194 - protein, 7 aa, synthetic construct
  REGION 1..7: note = human mAb CDR:LC2; clone: p1.22
GASRRAT

SEQ ID NO: 195 - protein, 7 aa, synthetic construct
  REGION 1..7: note = human mAb CDR:LC2; clone: p3.1.34
DASTRAT

SEQ ID NO: 196 - protein, 7 aa, synthetic construct
  REGION 1..7: note = human mAb CDR:LC2; clone: p3.1.35
GANTRAT

SEQ ID NO: 197 - protein, 7 aa, synthetic construct
  REGION 1..7: note = human mAb CDR:LC2; clone: p3.1.36
DTSTRAT

SEQ ID NO: 198 - protein, 7 aa, synthetic construct
  REGION 1..7: note = human mAb CDR:LC2; clone: p3.1.37
DASTRAT

SEQ ID NO: 199 - protein, 7 aa, synthetic construct
  REGION 1..7: note = human mAb CDR:LC2; clone: p3.2.38
GANTRAT

SEQ ID NO: 200 - protein, 7 aa, synthetic construct
  REGION 1..7: note = human mAb CDR:LC2; clone: p3.2.40
DTSTRAT

SEQ ID NO: 201 - protein, 8 aa, synthetic construct
  REGION 1..8: note = human mAb CDR:LC3; clone: p2.3
QQYSNWPP

SEQ ID NO: 202 - protein, 8 aa, synthetic construct
  REGION 1..8: note = human mAb CDR:LC3; clone: p2.8
QQYNTWPP

SEQ ID NO: 203 - protein, 8 aa, synthetic construct
  REGION 1..8: note = human mAb CDR:LC3; clone: p2.9
QQYYDWPP

SEQ ID NO: 204 - protein, 8 aa, synthetic construct
  REGION 1..8: note = human mAb CDR:LC3; clone: p2.10
QQYYDWPP

SEQ ID NO: 205 - protein, 8 aa, synthetic construct
  REGION 1..8: note = human mAb CDR:LC3; clone: p2.4
QQYYDWPP

SEQ ID NO: 206 - protein, 8 aa, synthetic construct
  REGION 1..8: note = human mAb CDR:LC3; clone: p2.11
QQSYDWPP

SEQ ID NO: 207 - protein, 8 aa, synthetic construct
  REGION 1..8: note = human mAb CDR:LC3; clone: p2.12
QQYNNWPP

SEQ ID NO: 208 - protein, 8 aa, synthetic construct
  REGION 1..8: note = human mAb CDR:LC3; clone: p2.13
QQSYDWPP

SEQ ID NO: 209 - protein, 8 aa, synthetic construct
  REGION 1..8: note = human mAb CDR:LC3; clone: p2.14
QQYNNWPP

SEQ ID NO: 210 - protein, 8 aa, synthetic construct
  REGION 1..8: note = human mAb CDR:LC3; clone: p2.15
QQYYDWPP

SEQ ID NO: 211 - protein, 8 aa, synthetic construct
  REGION 1..8: note = human mAb CDR:LC3; clone: p2.16
QQYSNWPP

SEQ ID NO: 212 - protein, 8 aa, synthetic construct
  REGION 1..8: note = human mAb CDR:LC3; clone: p2.17
QQYYDWPP

SEQ ID NO: 213 - protein, 8 aa, synthetic construct
  REGION 1..8: note = human mAb CDR:LC3; clone: p2.18
QQYYDWPP

SEQ ID NO: 214 - protein, 8 aa, synthetic construct
  REGION 1..8: note = human mAb CDR:LC3; clone: p2.19
QQYNNWPP

SEQ ID NO: 215 - protein, 8 aa, synthetic construct
  REGION 1..8: note = human mAb CDR:LC3; clone: p2.20
QQYNNWPP

SEQ ID NO: 216 - protein, 8 aa, synthetic construct
  REGION 1..8: note = human mAb CDR:LC3; clone: p2.21
QQSYDWPP

SEQ ID NO: 217 - protein, 8 aa, synthetic construct
  REGION 1..8: note = human mAb CDR:LC3; clone: p1.22
QQYYDWPP

SEQ ID NO: 218 - protein, 8 aa, synthetic construct
  REGION 1..8: note = human mAb CDR:LC3; clone: p1.23
QQYYDWPP

SEQ ID NO: 219 - protein, 8 aa, synthetic construct
  REGION 1..8: note = human mAb CDR:LC3; clone: p1.24
QQYYDWPP

SEQ ID NO: 220 - protein, 8 aa, synthetic construct
  REGION 1..8: note = human mAb CDR:LC3; clone: p1.25
QQYYDWRP

SEQ ID NO: 221 - protein, 8 aa, synthetic construct
  REGION 1..8: note = human mAb CDR:LC3; clone: p1.1
QQYYVWPP

SEQ ID NO: 222 - protein, 8 aa, synthetic construct
  REGION 1..8: note = human mAb CDR:LC3; clone: p1.26
QQYYVWPP

SEQ ID NO: 223 - protein, 8 aa, synthetic construct
  REGION 1..8: note = human mAb CDR:LC3; clone: p1.27
QQYYDWPP

SEQ ID NO: 224 - protein, 8 aa, synthetic construct
  REGION 1..8: note = human mAb CDR:LC3; clone: p1.28
QQYYDWPP

SEQ ID NO: 225 - protein, 8 aa, synthetic construct
  REGION 1..8: note = human mAb CDR:LC3; clone: p1.29
QQYNNWPP

SEQ ID NO: 226 - protein, 8 aa, synthetic construct
  REGION 1..8: note = human mAb CDR:LC3; clone: p1.30
QQYYDWPP

SEQ ID NO: 227 - protein, 8 aa, synthetic construct
  REGION 1..8: note = human mAb CDR:LC3; clone: p1.6
QQYYDWPP

SEQ ID NO: 228 - protein, 8 aa, synthetic construct
  REGION 1..8: note = human mAb CDR:LC3; clone: p1.2
QQYMTWPP

SEQ ID NO: 229 - protein, 8 aa, synthetic construct
  REGION 1..8: note = human mAb CDR:LC3; clone: p1.31
QQYMTWPP

SEQ ID NO: 230 - protein, 8 aa, synthetic construct
  REGION 1..8: note = human mAb CDR:LC3; clone: p1.5
QQYNNWPP

SEQ ID NO: 231 - protein, 8 aa, synthetic construct
  REGION 1..8: note = human mAb CDR:LC3; clone: p1.32
QQYYDWPP

SEQ ID NO: 232 - protein, 8 aa, synthetic construct
  REGION 1..8: note = human mAb CDR:LC3; clone: p1.33
QQYYDWRP

SEQ ID NO: 233 - protein, 8 aa, synthetic construct
  REGION 1..8: note = human mAb CDR:LC3; clone: p1.39
QQYMTWPP

SEQ ID NO: 234 - protein, 8 aa, synthetic construct
  REGION 1..8: note = human mAb CDR:LC3; clone: p1.22
QQYYDWPP

SEQ ID NO: 235 - protein, 8 aa, synthetic construct
  REGION 1..8: note = human mAb CDR:LC3; clone: p3.1.34
QQYYDWPP

SEQ ID NO: 236 - protein, 8 aa, synthetic construct
  REGION 1..8: note = human mAb CDR:LC3; clone: p3.1.35
QQYYDWPP

SEQ ID NO: 237 - protein, 8 aa, synthetic construct
  REGION 1..8: note = human mAb CDR:LC3; clone: p3.1.36
QQYYDWPP

SEQ ID NO: 238 - protein, 8 aa, synthetic construct
  REGION 1..8: note = human mAb CDR:LC3; clone: p3.1.37
QQYYDWPP

SEQ ID NO: 239 - protein, 8 aa, synthetic construct
  REGION 1..8: note = human mAb CDR:LC3; clone: p3.2.38
QQYYDWPP

SEQ ID NO: 240 - protein, 8 aa, synthetic construct
  REGION 1..8: note = human mAb CDR:LC3; clone: p3.2.40
QQYYDWPP

SEQ ID NO: 241 - protein, 1124 aa, Homo sapiens
MDSLASLVLCGVSLLLSGTVEGAMDLILINSLPLVSDAETSLTCIASGWRPHEPITIGRDFEALMNQHQDPLEVTQDVTREWAKKVVWKREKASKINGAYFCEGRVRGEAIRIRTMKMRQQASFLPATLTMTVDKGDNVNISFKKVLIKEEDAVIYKNGSFIHSVPRHEVPDILEVHLPHAQPQDAGVYSARYIGGNLFTSAFTRLIVRRCEAQKWGPECNHLCTACMNNGVCHEDTGECICPPGFMGRTCEKACELHTFGRTCKERCSGQEGCKSYVFCLPDPYGCSCATGWKGLQCNEACHPGFYGPDCKLRCSCNNGEMCDRFQGCLCSPGWQGLQCEREGIQRMTPKIVDLPDHIEVNSGKFNPICKASGWPLPTNEEMTLVKPDGTVLHPKDFNHTDHFSVAIFTIHRILPPDSGVWVCSVNTVAGMVEKPFNISVKVLPKPLNAPNVIDTGHNFAVINISSEPYFGDGPIKSKKLLYKPVNHYEAWQHIQVTNEIVTLNYLEPRTEYELCVQLVRRGEGGEGHPGPVRRFTTASIGLPPPRGLNLLPKSQTTLNLTWQPIFPSSEDDFYVEVERRSVQKSDQQNIKVPGNLTSVLLNNLHPREQYVVRARVNTKAQGEWSEDLTAWTLSDILPPQPENIKISNITHSSAVISWTILDGYSISSITIRYKVQGKNEDQHVDVKIKNATITQYQLKGLEPETAYQVDIFAENNIGSSNPAFSHELVTLPESQAPADLGGGKMLLIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 242 - protein, 122 aa, synthetic construct
  REGION 1..122: note = human mab p1.1- VH
DVQLVESGGGVVRPGESLRLSCAASGFTFSSYAMSWVRQAPGEGLEWVARISDYTIYYADSVKGRFTISRDNSKNTLYLQMNSLRAEDTAVYYCAKSGPKSGKYWNNFFDSWGLGTLVTVSS

SEQ ID NO: 243 - protein, 108 aa, synthetic construct
  REGION 1..108: note = human mab p1.1 - VL
EIVLTQSPGTLSVSPGERATLTCRASQSVKTDLAWYQQKPGQAPRLLIYGATTRATGIPARFSGSGSGTEFTLTITNLQSEDFAVYYCQQYYVWPPLTFGGGTKVEIK

SEQ ID NO: 244 - protein, 120 aa, synthetic construct
  REGION 1..120: note = human Mab p1.2 - VH
DVQLVESGGGVVRPGESLRLSCAASGFSFINFAMTWVRQAPGEGPEWVSLISDDGRGNRPYYADSVKGRFTISRDNSKNILYLQMNSLRVEDTATYYCAKVSWDVFFDYWGLGTVVTVSS

SEQ ID NO: 245 - protein, 108 aa, synthetic construct
  REGION 1..108: note = human Mab p1.2 VL
EIVLTQSPGTVSLSPGERVTLSCRASQPIDVYLAWYQQKPGQAPRLLIYGANRRAIDIPDRFSGSGSGTEFTLTINSLQNEDFAVYFCQQYMTWPPVTFGGGTKVEIK

SEQ ID NO: 246 - protein, 115 aa, synthetic construct
  REGION 1..115: note = human Mab p2.3 - VH
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSS

SEQ ID NO: 247 - protein, 108 aa, synthetic construct
  REGION 1..108: note = human Mab p2.3 - VL
EIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIK

SEQ ID NO: 248 - protein, 115 aa, synthetic construct; identical to SEQ ID NO: 246
  REGION 1..115: note = human mAb p4.1.7 - VH

SEQ ID NO: 249 - protein, 108 aa, synthetic construct
  REGION 1..108: note = human mAb p4.1.7 - VL
EIVLTQSPGTLSLSPGERATLSCRASQSVSSNLAWYQQKPGQAPRLLIYDASSRATGIPARFSGSGSGTEFTLTISSLQSEDFALYYCQQYNNWPPLTFGGGTKVEIK

SEQ ID NO: 250 - protein, 117 aa, synthetic construct
  REGION 1..117: note = human mAb p4.3.41 - VH
DVQLVESGGGVVRPGESLRLSCAASGFTFSIYAMNWVRQAPGEGLEWISGISGSGALTYYADSVKGRFTISRDNSKNTLYLQMNSLRAEDTAVYYCAKPLNNFFDYWGLGTLVTVSS

SEQ ID NO: 251 - protein, 108 aa, synthetic construct
  REGION 1..108: note = human mAb p4.3.41 - VL
EIVLTQSPGTLSLSPGERATLSCRASQSVGSKLAWYQQKPGQAPRLLIFYASSRATGIPDRFSGSGSGTDFTLTIRSLQSEDFAVYYCQQYHDWPPLTFGGGTKVEIK

SEQ ID NO: 252 - protein, 115 aa, synthetic construct; identical to SEQ ID NO: 246
  REGION 1..115: note = human mAb p4.3.42 - VH

SEQ ID NO: 253 - protein, 108 aa, synthetic construct
  REGION 1..108: note = human mAb p4.3.42 - VL
EIVLTQSPGTLSSSPGERATLSCRASQTVGSYLAWYQQKPGQAPRLLIYAASSRATGIPDRFSGSGSGTEFTLTISSLQSEDFAVYYCQQYKDWPPLTFGGGTKVEIK

SEQ ID NO: 254 - protein, 118 aa, synthetic construct
  REGION 1..118: note = human mAb p5.43 - VH
DVQLVESGGGLVQPGESLRLSCTASGFTFPTYALSWVRQAPGEGLEWVSSTIWGGDTTYYAESVKGRFIISRDSSRNTLYLQMNSLRAEDTAVYYCAQGTWNIFFDYWGLGVLVTVSS

SEQ ID NO: 255 - protein, 108 aa, synthetic construct
  REGION 1..108: note = human mAb p5.43 - VL
EIVLTQSPGTLSLSPGERATLSCRASQGIAGNLAWYQQKPGQAPRLLIYGASSRATGIPDRFSGSGSGTEFTLTISSLQSEDFAVYYCQQYNNWPPLTFGGGTKVEIK

SEQ ID NO: 256 - protein, 117 aa, synthetic construct
  REGION 1..117: note = human mAb p4.2.44 - VH
DVQLVESGGGVVRPGESLRLSCAASGFSFSLYAMNWVRQAPGEGLEWVSRISGRDGYTYYADSVKGRFTVSRDNSKNTLYLQMNNLRAEDTAVYYCAKTLNNFFDYWGLGTLVTVSL

SEQ ID NO: 257 - protein, 109 aa, synthetic construct
  REGION 1..109: note = human mAb p4.2.44 - VL
EIVLTQSPGTLILSPGERATLSCRASQSISNTYLAWYQQKPGQAPRLLIYDASNRAADIPDRFSGSGSGTDFTLTITRLEPEDFAVYYCQQYYDWPPLTFGGGTKVEIK

SEQ ID NO: 258 - protein, 114 aa, synthetic construct
  REGION 1..114: note = human mAb p5.45 VH
DVQLVESGGGVVRPGESLTLSCAASGFTFSSYGMSWVRQAPGEGLEWVSAINGGSTFYADSVKGRFTISRDNSKNILYLQMNSLRAEDTAIYYCANWNSFFDYWGLGTLVTVSS

SEQ ID NO: 259 - protein, 108 aa, synthetic construct
  REGION 1..108: note = human mAb p5.45 VL
EIVLTQSPGTLSLSPGERATLTCRASQHVNTHVAWYQQRPGQAPRLIIYDATNRANDVPDRFSGSGSGTDFTLTISSLEPEDFAIYYCQQYNNWPPLTFGGGTKVEIK

SEQ ID NO: 260 - protein, 114 aa, synthetic construct
  REGION 1..114: note = human mAb p5.46 - VH
DVQLVESGGGVVRPGESLTLSCVASGFTFSSYGMSWVRQAPGEGLEWVSAINGGSTFYADSVKGRFTISRDNSKNILYLQMNSLRAEDTAIYYCANWNSFFDYWGLGTLVTVSS

SEQ ID NO: 261 - protein, 108 aa, synthetic construct; identical to SEQ ID NO: 259
  REGION 1..108: note = human mAb p5.46 - VL

SEQ ID NO: 262 - protein, 119 aa, synthetic construct
  REGION 1..119: note = human mAb p1.6 - VH
DVQLVESGGGVVRPGESLRLSCTASGFSFDVYAMTWVRQAPGKGLEWLTLISGTGRMTYYADSVKGRFTVSRDNSKKTLYLQMNSLRAEDTAVYYCAKDPFGHGFFDYWGLGTLVTVSS

SEQ ID NO: 263 - protein, 108 aa, synthetic construct
  REGION 1..108: note = human mAb p1.6 - VL
EIVLTQSPGTLTLSPGETATLTCRASQSVNRNLAWYQEKPDQAPRLLIYDARTRATGVPDRFSGSGSGTDFTLTINSLQSEDFAVYVCQQYYDWPPFTFGGGTKVEIK

SEQ ID NO: 264 - protein, 122 aa, synthetic construct
  REGION 1..122: note = human mAb p4.2.47 - VH
DVQLVESGGGEVRPGEPLRLSCAASGFTFNTYGMSWVRQAPGEGLEWVSVISGSGGSTYHADSVKGRFTISRDNSKNTLYLQMNSLRVEDTAIYYCAKDSGSWTSSGFFDYWGLGTLVTVSS

SEQ ID NO: 265 - protein, 108 aa, synthetic construct
  REGION 1..108: note = human mAb p4.2.47 - VL
EIVLTQSPGTLSVSPGEKATLNCRASQNINSKLAWYQQKPGQAPRLLIYDAGNRAAGIPDRFSGSGSGTEFTLTISSLQSEDFAVYYCQQYYDWRPLTFGGGTKVEIK

SEQ ID NO: 266 - protein, 117 aa, synthetic construct
  REGION 1..117: note = human mAb p5.48 - VH
DVQLVESGGAVVRPGESLRLSCAASGFSFDIYGMNWVRQAPGKGLEWVSRISGNGGSTFYSESVKGRFTISRDNSKSTLYLQMNSLRAEDTAIYYCARTLNNFFDYWGLGTLVTVSS

SEQ ID NO: 267 - protein, 108 aa, synthetic construct
  REGION 1..108: note = human mAb p5.48 - VL
EIVLTQSPGTLSVSPGERATLSCRASQHVGGNLAWYQQKPGQAPRLLIYDASNRATGIPDRFSGSGSGTDFSLTISRLEPEDFAVYYCQQYYDWPPLTFGGGTKVEIK

SEQ ID NO: 268 - protein, 119 aa, synthetic construct; identical to SEQ ID NO: 262
  REGION 1..119: note = human mAb p1.5 - VH

SEQ ID NO: 269 - protein, 108 aa, synthetic construct
  REGION 1..108: note = human mAb p1.5 - VL
EIVLTQSPGTLSLSPGERATLSCRASQTVGSKLAWYQQKPGQAPRLLIYDASTRATGIPDRFSGSGSGTDFTLTISSLQSEDFAVYYCQQYNNWPPLTFGGGTKVEIK

SEQ ID NO: 270 - protein, 120 aa, synthetic construct
  REGION 1..120: note = human mAb p3.2.49 - VH
DVQLVESGGGVVRPGESLRLSCSASGFTFDIYAMTWVRQAPGEGLEWVSTLTGISGRGGSTFYADSVKGRFTISRDSSDNILYLQMNSLRAEDTAIYYCAGGTDIFFDYWGLGILVTVSS

SEQ ID NO: 271 - protein, 108 aa, synthetic construct
  REGION 1..108: note = human mAb p3.2.49 - VL
EMVLTQSPGTLSLSPGERATLSCRASQSVGNYLAWYQQKPGQAPRLLIYDASSRATGIPARFSGSGSGTEFTLTISSLQSEDFAVYYCQQYNNWPPLTFGGGTKVEIK

SEQ ID NO: 272 - protein, 120 aa, synthetic construct
  REGION 1..120: note = human mAb p1.31 - VH
DVQLVESGGGVVRPGESPRLSCAASGFSFINFAMTWVRQAPGEGPEWVSLISDDGRGNRPYYADSVKGRFTISRDNSKNILYLQMNSLRVEDTATYYCAKVSWDVFFDYWGLGTLVTVSS

SEQ ID NO: 273 - protein, 108 aa, synthetic construct; identical to SEQ ID NO: 245
  REGION 1..108: note = human mAb p1.31 - VL

SEQ ID NO: 274 - protein, 445 aa, synthetic construct
  REGION 1..445: note = Artificial p2.3 with pro-hexamerizing and ADCC/CDC abrogating Fc mutations (heavy chain)
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 275 - protein, 215 aa, synthetic construct
  REGION 1..215: note = Artificial p2.3 with pro-hexamerizing and ADCC/CDC abrogating Fc mutations (light chain (kappa))
EIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 696 aa, synthetic construct
  REGION 1..696: note = Artificial p2.3 IgG-scFv fusion tetravalent mAb with HC C-terminal p2.3-scFv and ADCC/CDC abrogating Fc mutations (heavy chain)
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKGQSSRSSGGGGSSGGGGSDVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSS

SEQ ID NO: 277 - protein, 215 aa, synthetic construct; identical to SEQ ID NO: 275
  REGION 1..215: note = Artificial p2.3 IgG-scFv fusion tetravalent mAb with HC C-terminal p2.3-scFv and ADCC/CDC abrogating Fc mutations (light chain (kappa))

SEQ ID NO: 278 - protein, 696 aa, synthetic construct
  REGION 1..696: note = Artificial p2.3 IgG-scFv fusion tetravalent mAb with HC N-terminal p2.3-scFv and ADCC/CDC abrogating Fc mutations (heavy chain)
EIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKGQSSRSSGGGGSSGGGGSDVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSGGGGSGGGGSDVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 215 aa, synthetic construct; identical to SEQ ID NO: 275
  REGION 1..215: note = Artificial p2.3 IgG-scFv fusion tetravalent mAb with HC N-terminal p2.3-scFv and ADCC/CDC abrogating Fc mutations (light chain (kappa))

SEQ ID NO: 280 - protein, 701 aa, synthetic construct
  REGION 1..701: note = Artificial p2.3 IgG-scFv fusion biparatopic mAb with HC C-terminal p1.2-scFv and ADCC/CDC abrogating Fc mutations (heavy chain)
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEIVLTQSPGTVSLSPGERVTLSCRASQPIDVYLAWYQQKPGQAPRLLIYGANRRAIDIPDRFSGSGSGTEFTLTINSLQNEDFAVYFCQQYMTWPPVTFGGGTKVEIKGQSSRSSGGGGSSGGGGSDVQLVESGGGVVRPGESLRLSCAASGFSFINFAMTWVRQAPGEGPEWVSLISDDGRGNRPYYADSVKGRFTISRDNSKNILYLQMNSLRVEDTATYYCAKVSWDVFFDYWGLGTVVTVSS

SEQ ID NO: 281 - protein, 215 aa, synthetic construct; identical to SEQ ID NO: 275
  REGION 1..215: note = Artificial p2.3 IgG-scFv fusion biparatopic mAb with HC C-terminal p1.2-scFv and ADCC/CDC abrogating Fc mutations (light chain (kappa))

SEQ ID NO: 282 - protein, 701 aa, synthetic construct
  REGION 1..701: note = Artificial p2.3 IgG-scFv fusion Tie2/VEGF bispecific mAb with HC C-terminal VEGF-scFv and ADCC/CDC abrogating Fc mutations (heavy chain)
DVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSDIQMTQSPSSLSASVGDRVTITCRASQVIRRSLAWYQQKPGKAPKLLIYAASNLASGVPSRFSGSGSGTDFTLTISSLQPEDFATYYCQQSNTSPLTFGQGTKVEIKGQSSRSSGGGGSSGGGGSEVQLVESGGGLVQPGGSLRLSCAASGFSINGSWIFWVRQAPGKGLEWVGAIWPFGGYTHYADSVKGRFTISADTSKNTAYLQMNSLRAEDTAVYYCARWGHSTSPWAMDYWGQGTLVTVSS

SEQ ID NO: 283 - protein, 215 aa, synthetic construct; identical to SEQ ID NO: 275
  REGION 1..215: note = Artificial p2.3 IgG-scFv fusion Tie2/VEGF bispecific mAb with HC C-terminal VEGF-scFv and ADCC/CDC abrogating Fc mutations (light chain (kappa))

SEQ ID NO: 284 - protein, 682 aa, synthetic construct
  REGION 1..682: note = Aflibercept-p2.3 scFv fusion Tie2/VEGF bispecific mAb with C-terminal p2.3-scFv
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKGQSSRSSGGGGSSGGGGSDVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSS

SEQ ID NO: 285 - protein, 682 aa, synthetic construct
  REGION 1..682: note = Aflibercept-p2.3 scFv fusion Tie2/VEGF bispecific mAb with C-terminal p2.3-scFv and ADCC/CDC abrogating Fc mutations
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSEIVLTQSPGTLSLSPGEIATLSCRASQNVRSDLAWYQQKPGQAPRLLIYDASNRATGIPDRFSGSGSGTDFTLTISSLQSEDFAVYYCQQYSNWPPLTFGGGTKVEIKGQSSRSSGGGGSSGGGGSDVQLVESGGGVVRPGESLRLSCTASGFTFDSYGMSWVRQAPGKGLEWVSSINVGDNTYYADSVRGRFIISRDSSRNTLYLQMNSLTAEDTAVYYCANWNSFFDYWGLGTLVTVSS

SEQ ID NO: 286 - protein, 696 aa, synthetic construct; identical to SEQ ID NO: 276
  REGION 1..696: note = p2.3-ranibizumab Tie2/VEGF bispecific mAb with HC C-terminal ranibizumab-scFv and ADCC/CDC abrogating Fc mutations (heavy chain)

SEQ ID NO: 287 - protein, 215 aa, synthetic construct; identical to SEQ ID NO: 275
  REGION 1..215: note = p2.3-ranibizumab Tie2/VEGF bispecific mAb with HC C-terminal ranibizumab-scFv and ADCC/CDC abrogating Fc mutations (light chain (kappa))

SEQ ID NO: 288 - protein, 696 aa, synthetic construct; identical to SEQ ID NO: 278
  REGION 1..696: note = p2.3-ranibizumab Tie2/VEGF bispecific mAb with HC N-terminal ranibizumab-scFv and ADCC/CDC abrogating Fc mutations (heavy chain)

SEQ ID NO: 289 - protein, 215 aa, synthetic construct; identical to SEQ ID NO: 275
  REGION 1..215: note = p2.3-ranibizumab Tie2/VEGF bispecific mAb with HC N-terminal ranibizumab-scFv and ADCC/CDC abrogating Fc mutations (light chain (kappa))

SEQ ID NO: 290 - protein, 50 aa, synthetic construct
  VARIANT 6..50: note = Wherein up to 9 copies of GGGGS may be omitted
GGGGSGGGGSGGGGSGGGGSGGGGSGGGGSGGGGSGGGGSGGGGSGGGGS

SEQ ID NO: 291 - protein, 50 aa, synthetic construct
  VARIANT 6..50: note = Wherein up to 9 copies of SSSSG may be omitted
SSSSGSSSSGSSSSGSSSSGSSSSGSSSSGSSSSGSSSSGSSSSGSSSSG

SEQ ID NO: 292 - protein, 54 aa, synthetic construct
  VARIANT 10..54: note = Wherein up to 9 copies of SGGGG may be omitted
GGGGSGGGGSGGGGSGGGGSGGGGSGGGGSGGGGSGGGGSGGGGSGGGGSGGGG

SEQ ID NO: 293 - protein, 50 aa, synthetic construct
  VARIANT 6..50: note = Wherein up to 9 copies of EAAAK may be omitted
EAAAKEAAAKEAAAKEAAAKEAAAKEAAAKEAAAKEAAAKEAAAKEAAAK

(19) United States
(12) Patent Application Publication
(10) Pub. No.: US 2024/0392012 A1
(43) Pub. Date: Nov. 28, 2024

(54) Antibodies Directed to TIE-2 and Methods of Use

(71) Applicant: Unity Biotechnology, Inc., South San Francisco, CA (US)

(72) Inventors: Jason Damiano, South San Francisco, CA (US); Kristina Oresic Bender, South San Francisco, CA (US); Richard Theolis, South San Francisco, CA (US); Deepika Bhatnagar, South San Francisco, CA (US); Nina Ly, South San Francisco, CA (US)

(21) Appl. No.: 18/596,448

(22) Filed: Mar. 5, 2024

Related U.S. Application Data
(62) Division of application No. 16/953,165, filed on Nov. 19, 2020, now abandoned.
(60) Provisional application No. 62/938,816, filed on Nov. 21, 2019.

(51) Int. Cl. C07K 16/28; A61K 31/713; A61P 27/02; C12N 5/10; C12N 15/63

(52) U.S. Cl. CPC C07K 16/2863; A61K 31/713; A61P 27/02; C12N 5/10; C12N 15/63; C07K 2317/21; C07K 2317/31; C07K 2317/33; C07K 2317/60; C07K 2317/622; C07K 2317/75; C07K 2317/92

(57) ABSTRACT

The invention relates to anti-Tie2 antibodies and methods of using the same.

### CROSS-REFERENCE TO RELATED APPLICATIONS

This application claims the benefit of priority to U.S. Provisional Application No. 62/938,816, filed Nov. 21, 2019, and to U.S. application Ser. No. 16/953,165, filed Nov. 19, 2020, the disclosures of which are incorporated herein by reference in their entirety.

### INCORPORATION BY REFERENCE OF SEQUENCE LISTING PROVIDED AS A SEQUENCE LISTING XML FILE

A Sequence Listing is provided herewith as a Sequence Listing XML, “UNITY-022DIV_SEQ_LIST_MAY_2024” created on May 20, 2024, and having a size of 382,024 bytes. The contents of the Sequence Listing XML are incorporated by reference herein in their entirety.

### FIELD OF THE INVENTION

The invention relates to anti-Tie2 antibodies and methods of using the same.

### BACKGROUND OF THE INVENTION

Tie2 is a receptor tyrosine kinase that is predominantly expressed on the surface of endothelial cells, where it plays a central role in vessel stability, survival and maturation (Suri, C., et al., 1996. Requisite Role of Angiopoietin-1, a Ligand for the TIE2 Receptor, during Embryonic Angiogenesis. Cell 87 (7): 1171-80); Thurston, G., et al., 1999. Leakage-Resistant Blood Vessels in Mice Transgenically Overexpressing Angiopoietin-1. Science 286 (5449): 2511-14.; Saharinen, et al., 2010. How Do Angiopoietins Tie with Vascular Endothelial Growth Factors? Current Opinion in Hematology; Augustin, et al., 2009. Control of Vascular Morphogenesis and Homeostasis through the Angiopoietin-Tie System. Nature Reviews. Molecular Cell Biology 10 (3): 165-77; Milam, et al., 2015. The Angiopoietin-Tie2 Signaling Axis in the Vascular Leakage of Systemic Inflammation. Tissue Barriers 3 (1-2)). Tie2 activity is tightly regulated by at least four soluble protein factors known as angiopoietins 1 to 4. Angiopoietin-1 (Ang1) and angiopoietin-2 (Ang2) are believed to be the predominant regulators of Tie2 function. Under normal physiological conditions, high Ang1 levels and low Ang2 levels maintain constitutive activation of the Tie2 signaling axis. Specifically, the Ang1 agonist ligand binds directly to the Tie2 receptor, leading to Tie2 clustering, autophosphorylation and downstream signaling events including the activation of the PI3-kinase/Akt and MAPK pathways.

Gene-targeting experiments have indicated that the Ang/Tie signaling system is needed for physiological and pathological remodeling of both lymphatic and blood vessels in embryonic, postnatal and adult mice (Eklund L, Kangas J, Saharinen P. Angiopoietin-Tie signaling in the cardiovascular and lymphatic systems. Clin Sci (Lond) 2017; 131: 87-103). In humans, an altered expression of angiopoietins is implicated in many vascular diseases (Saharinen P, Eklund L, Alitalo K. Therapeutic targeting of the angiopoietin-TIE pathway. Nat Rev Drug Discov 2017; 16: 635-61).

There remains a need for anti-Tie2 antibodies with improved properties as well as therapeutic and diagnostic uses thereof.

### SUMMARY OF THE INVENTION

The present invention provides anti-Tie2 antibodies and methods of using the same for therapeutic and diagnostic purposes. The anti-Tie2 antibodies of the invention demonstrate unique properties that make them particularly suitable for use in therapy.

Specifically contemplated as part of the disclosed invention is:

Embodiment 1. An isolated anti-Tie2 antibody, or an antigen-binding fragment thereof, comprising three heavy chain complementarity-determining regions (CDR H1-3) and three light chain CDRs (CDR L1-3) as follows:

| | HCDR1 | HCDR2 | HCDR3 | LCDR1 | LCDR2 | LCDR3 |
|---|---|---|---|---|---|---|
| Antibody | SEQ | SEQ | SEQ | SEQ | SEQ | SEQ |
| Clone # | ID No. | ID No. | ID No. | ID No. | ID No. | ID No. |
| 3 | 1 | 41 | 81 | 121 | 161 | 201 |
| 8 | 2 | 42 | 82 | 122 | 162 | 202 |
| 9 | 3 | 43 | 83 | 123 | 163 | 203 |
| 10 | 4 | 44 | 84 | 124 | 164 | 204 |
| 4 | 5 | 45 | 85 | 125 | 165 | 205 |
| 11 | 6 | 46 | 86 | 126 | 166 | 206 |
| 12 | 7 | 47 | 87 | 127 | 167 | 207 |
| 13 | 8 | 48 | 88 | 128 | 168 | 208 |
| 14 | 9 | 49 | 89 | 129 | 169 | 209 |
| 15 | 10 | 50 | 90 | 130 | 170 | 210 |
| 16 | 11 | 51 | 91 | 131 | 171 | 211 |
| 17 | 12 | 52 | 92 | 132 | 172 | 212 |
| 18 | 13 | 53 | 93 | 133 | 173 | 213 |
| 19 | 14 | 54 | 94 | 134 | 174 | 214 |
| 20 | 15 | 55 | 95 | 135 | 175 | 215 |
| 21 | 16 | 56 | 96 | 136 | 176 | 216 |
| 22 | 17 | 57 | 97 | 137 | 177 | 217 |
| 23 | 18 | 58 | 98 | 138 | 178 | 218 |
| 24 | 19 | 59 | 99 | 139 | 179 | 219 |
| 25 | 20 | 60 | 100 | 140 | 180 | 220 |
| 1 | 21 | 61 | 101 | 141 | 181 | 221 |
| 26 | 22 | 62 | 102 | 142 | 182 | 222 |
| 27 | 23 | 63 | 103 | 143 | 183 | 223 |
| 28 | 24 | 64 | 104 | 144 | 184 | 224 |
| 29 | 25 | 65 | 105 | 145 | 185 | 225 |
| 30 | 26 | 66 | 106 | 146 | 186 | 226 |
| 6 | 27 | 67 | 107 | 147 | 187 | 227 |
| 2 | 28 | 68 | 108 | 148 | 188 | 228 |
| 31 | 29 | 69 | 109 | 149 | 189 | 229 |
| 5 | 30 | 70 | 110 | 150 | 190 | 230 |
| 32 | 31 | 71 | 111 | 151 | 191 | 231 |
| 33 | 32 | 72 | 112 | 152 | 192 | 232 |
| 39 | 33 | 73 | 113 | 153 | 193 | 233 |
| 22 | 34 | 74 | 114 | 154 | 194 | 234 |
| 34 | 35 | 75 | 115 | 155 | 195 | 235 |
| 35 | 36 | 76 | 116 | 156 | 196 | 236 |
| 36 | 37 | 77 | 117 | 157 | 197 | 237 |
| 37 | 38 | 78 | 118 | 158 | 198 | 238 |
| 38 | 39 | 79 | 119 | 159 | 199 | 239 |
| 40 | 40 | 80 | 120 | 160 | 200 | 240 |

Embodiment 2. An isolated anti-Tie2 antibody, or an antigen-binding fragment thereof comprising:
a heavy chain variable (VH) domain comprising an amino acid sequence of SEQ ID NO.242 and a light chain variable (VL) domain comprising an amino acid sequence of SEQ ID NO.243;
a heavy chain variable (VH) domain comprising an amino acid sequence of SEQ ID NO.244 and a light chain variable (VL) domain comprising an amino acid sequence of SEQ ID NO.245;
a heavy chain variable (VH) domain comprising an amino acid sequence of SEQ ID NO.246 and a light chain variable (VL) domain comprising an amino acid sequence of SEQ ID NO.247;
a heavy chain variable (VH) domain comprising an amino acid sequence of SEQ ID NO.248 and a light chain variable (VL) domain comprising an amino acid sequence of SEQ ID NO.249;
a heavy chain variable (VH) domain comprising an amino acid sequence of SEQ ID NO.250 and a light chain variable (VL) domain comprising an amino acid sequence of SEQ ID NO.251;
a heavy chain variable (VH) domain comprising an amino acid sequence of SEQ ID NO.252 and a light chain variable (VL) domain comprising an amino acid sequence of SEQ ID NO.253;
a heavy chain variable (VH) domain comprising an amino acid sequence of SEQ ID NO.254 and a light chain variable (VL) domain comprising an amino acid sequence of SEQ ID NO.255;
a heavy chain variable (VH) domain comprising an amino acid sequence of SEQ ID NO.256 and a light chain variable (VL) domain comprising an amino acid sequence of SEQ ID NO.257;
a heavy chain variable (VH) domain comprising an amino acid sequence of SEQ ID NO.258 and a light chain variable (VL) domain comprising an amino acid sequence of SEQ ID NO.259;
a heavy chain variable (VH) domain comprising an amino acid sequence of SEQ ID NO.260 and a light chain variable (VL) domain comprising an amino acid sequence of SEQ ID NO.261;
a heavy chain variable (VH) domain comprising an amino acid sequence of SEQ ID NO.262 and a light chain variable (VL) domain comprising an amino acid sequence of SEQ ID NO.263;
a heavy chain variable (VH) domain comprising an amino acid sequence of SEQ ID NO.264 and a light chain variable (VL) domain comprising an amino acid sequence of SEQ ID NO.265;
a heavy chain variable (VH) domain comprising an amino acid sequence of SEQ ID NO.266 and a light chain variable (VL) domain comprising an amino acid sequence of SEQ ID NO.267;
a heavy chain variable (VH) domain comprising an amino acid sequence of SEQ ID NO.268 and a light chain variable (VL) domain comprising an amino acid sequence of SEQ ID NO.269; or
a heavy chain variable (VH) domain comprising an amino acid sequence of SEQ ID NO.270 and a light chain variable (VL) domain comprising an amino acid sequence of SEQ ID NO.271.

Embodiment 3. An isolated anti-Tie2 antibody, wherein the antibody specifically binds to an epitope within the human Tie2 extracellular domain, said epitope comprising amino acid residues K312, S316, C332, H358, K387, and T391, according to EU numbering as in Kabat, as measured by crosslink mass spectrometry.

Embodiment 4. The antibody of embodiments 1-3, wherein the antibody is an allosteric activator of Tie2.

Embodiment 5. The antibody of embodiments 1-4, wherein the antibody is a non-ligand competitive binder of Tie2.

Embodiment 6. The antibody of embodiments 3-5, wherein the antibody is cross-reactive against human, mouse, rat, rabbit and monkey Tie2.

Embodiment 7. The antibody of embodiments 1-6, wherein said antibody is fully human, humanized, monoclonal, or chimeric.

Embodiment 8. The antibody of embodiments 1-7, wherein said antibody is monospecific.

Embodiment 9. The antibody of embodiments 1-7, wherein said antibody is multispecific.

Embodiment 10. The antibody of embodiment 9, wherein the multispecific antibody is bispecific.

Embodiment 11. The antibody of embodiment 10, wherein the bispecific antibody comprises one binding arm that specifically binds human Tie-2 of claim 8 and a second binding arm that specifically binds VEGF-A, VEGF-B, VEGF-C, VEGF variants, Ang-1,Ang-2, Ang-3, Ang-4, PDGF-β, interleukin-1β, VE-PTP, complement factor C3, integrin α5β1, amyloid beta, PD-1, PD-L1, or CTLA-4.

Embodiment 12. The antibody of embodiment 9, wherein the multispecific antibody is a biparatopic antibody.

Embodiment 13. The antibody of embodiment 12, wherein the biparatopic antibody comprises one binding arm that specifically binds a first epitope on the ECD of human Tie2 and the other binding arm that specifically binds to a second epitope on the ECD of human Tie2.

Embodiment 14. The antibody of embodiment 9, wherein the multispecific antibody is a trivalent, a tetravalent, a pentavalent, a hexavalent antibody, wherein the trivalent, tetravalent, pentavalent, or hexavalent antibody comprises at least one binding arm that specifically binds human Tie2 of claim 8 and other remaining binding arms that specifically binds VEGF-A, VEGF-B, VEGF-C, VEGF variants, Ang-1, Ang-2, Ang-3, Ang-4, PDGF-β, interleukin-1β, VE-PTP, complement factor C3, integrin α5β1, amyloid beta, PD-1, PD-L1, or CTLA-4.

Embodiment 15. The antibody of embodiments 1-14, wherein said antibody is an antibody fragment that specifically binds human Tie2.

Embodiment 16. The antibody of embodiment 15, wherein the antibody fragment is a Fab, a Fab′-SH, a Fv, a scFv, or a (Fab′)₂ fragment.

Embodiment 17. The antibody of embodiment 16, wherein the multispecific antibody is comprised of scFv antibody fragments linked together by a polypeptide linker.

Embodiment 18. The antibody of embodiments 1-17, wherein the antibody possesses reduced effector function.

Embodiment 19. The antibody of embodiment 18, wherein the antibody comprises at least one substitution mutation at amino acid residue N297, L234, L235, P329, D265, and E430 according to EU numbering as in Kabat.

Embodiment 20. The antibody of embodiment 19, wherein the at least one substitution mutation is selected from the group consisting of amino acid residue N297G, N297A, L234A, L235A, P329G, D265A, and E430G according to EU numbering as in Kabat.

Embodiment 21. The antibody of embodiment 20, wherein the antibody comprises the substitution mutation at residue N297A or N297G.

Embodiment 22. The antibody of embodiment 20, wherein the antibody comprises the substitution mutation at residues L234A, L235A and P329G.

Embodiment 23. The antibody of embodiment 20, wherein the antibody comprises the substitution mutation at residues D265A and N297G.

Embodiment 24. The antibody of embodiment 21, wherein the antibody further comprises the substitution mutation at residue E430G.

Embodiment 25. The antibody of embodiment 22, wherein the antibody further comprises the substitution mutation at residue E430G.

Embodiment 26. The antibody of embodiment 23, wherein the antibody further comprises the substitution mutation at residue E430G.

Embodiment 27. The antibody of embodiment 25, wherein the antibody comprises a heavy chain comprising an amino acid sequence of SEQ ID NO:174 and a light chain comprising an amino acid sequence of SEQ ID NO:175.

Embodiment 28. The antibody of embodiment 22 comprising:
a heavy chain comprising an amino acid sequence of SEQ ID NO:276 and a light chain comprising an amino acid sequence of SEQ ID NO:277;
a heavy chain comprising an amino acid sequence of SEQ ID NO:278 and a light chain comprising an amino acid sequence of SEQ ID NO:279;
a heavy chain comprising an amino acid sequence of SEQ ID NO:280 and a light chain comprising an amino acid sequence of SEQ ID NO:281;
a heavy chain comprising an amino acid sequence of SEQ ID NO:282 and a light chain comprising an amino acid sequence of SEQ ID NO:283;
a heavy chain comprising an amino acid sequence of SEQ ID NO:286 and a light chain comprising an amino acid sequence of SEQ ID NO:287; or
a heavy chain comprising an amino acid sequence of SEQ ID NO:288 and a light chain comprising an amino acid sequence of SEQ ID NO:289.

Embodiment 29. The antibody of embodiment 10 comprising:
an amino acid sequence of SEQ ID NO:284; or
an amino acid sequence of SEQ ID NO:285.

Embodiment 30. An isolated nucleic acid encoding the antibody of embodiments 1-29.

Embodiment 31. A vector comprising the isolated nucleic acid of embodiment 30.

Embodiment 32. A host cell comprising the vector of embodiment 31.

Embodiment 33. A method of producing the antibody of embodiments 1-29, the method comprising culturing the host cell of embodiment 32 in a culture medium and isolating the resulting antibody.

Embodiment 34. An immunoconjugate comprising the antibody of embodiments 1-29.

Embodiment 35. A fusion polypeptide comprising the antibody of embodiments 1-29.

Embodiment 36. A pharmaceutical composition comprising the antibody of embodiments 1-29, the immunoconjugate of embodiment 34, or the fusion polypeptide of embodiment 35.

Embodiment 37. The pharmaceutical composition of embodiment 36, wherein the antibody, the immunoconjugate, or the fusion polypeptide is co-formulated with an anti-VEGF antibody or a VEGF extracellular trap protein.

Embodiment 38. A method of treating a Tie2 dysregulated disease in a subject in need thereof, the method comprising administering to the subject the pharmaceutical composition of embodiment 36.

Embodiment 39. A method of treating a Tie2 dysregulated disease in a subject in need thereof, the method comprising administering to the subject the pharmaceutical composition of embodiment 37.

Embodiment 40. The method of embodiment 38, further comprising co-administering to the subject a pharmaceutical composition comprising an anti-VEGF antibody or a VEGF extracellular trap protein.

Embodiment 41. The method of embodiments 38-40, wherein the Tie2 dysregulated disease comprise infectious diseases, acute respiratory distress syndrome (ARDS), ischemic injuries, ocular disorders, radiation injury, cancer, systemic sclerosis, traumatic brain injury, neuroinflammation, radiation injury, wound healing, myocardial infarction, blood brain barrier compromise, cerebral cavernous malformations, Duchenne Muscular dystrophy (DMD) or Clarkson Disease.

Embodiment 42. The method of embodiment 38-40, wherein the Tie2 dysregulated infectious diseases comprise sepsis, dengue virus infection, tuberculosis, or influenza.

Embodiment 43. The method of embodiment 38-40, wherein the Tie2 dysregulated ischemic injuries comprise diabetic nephropathy, acute kidney injury, chronic kidney disease, organ transplant, critical limb ischemia, traumatic brain injury or stroke.

Embodiment 44. The method of embodiment 38-40, wherein the Tie2 dysregulated ocular disorders comprise diabetic retinopathy, diabetic macular edema (DME), proliferative diabetic retinopathy (PDR) age-related macular degeneration (AMD), retinopathy of prematurity (ROP), or glaucoma.

Embodiment 45. The isolated anti-Tie2 antibody of embodiments 1-29, or the immunoconjugate of embodiment 34, or the fusion polypeptide of embodiment 35 for use in the treatment of the Tie2 dysregulated diseases of embodiments 41-44.

Embodiment 46. Use of the isolated anti-Tie2 antibody of embodiments 1-29, or the immunoconjugate of embodiment 34, or the fusion polypeptide of embodiment 35 for the manufacture of a medicament for treating the Tie2 dysregulated diseases of embodiments 41-44.

### BRIEF DESCRIPTION OF THE DRAWINGS

FIGS. 1A to 1C. Anti-Tie2 antibodies increase the levels of phospho-Tie2 (pTie2) as well as phospho-Akt (pAkt) and phospho-ERK (PERK) as determined by western blotting. HUVEC cells were serum starved and then treated with either PBS, Ang1 (12 nM), control hIgG1 (12 nM), or human anti-Tie2 antibodies #1, #2 and #3 or a wild-type (WT) chicken anti-Tie2 antibody (12 nM) for 20 minutes, followed by cell lysis. FIG. 1A shows Tie2 was immunoprecipitated from lysates using anti-Tie2 antibodies and levels of pTie2 were determined by western blotting with an anti-phospho-tyrosine antibody. For pAkt and pERK analysis, western blotting for pAkt, and total Akt (FIG. 1B) or pERK and total ERK (FIG. 1C) in whole cell lysates was performed. See Example 3.

FIG. 2. Functional potencies of anti-Tie2 antibodies are comparable to Ang1 in vitro. HUVEC cells were serum starved and treated with Ang1, human anti-Tie2 antibodies #1, #2, and #3, or WT chicken anti-Tie2 antibody at varying concentrations in nM, followed by cell lysis. Western blotting using pAkt-or total Akt-specific antibodies was performed. pAkt and total Akt signal intensity was measured using a LI-COR scanning fluorometer and levels of pAkt normalized to total Akt (pAkt/Akt ratio) were plotted on Y-axis using Graphpad™ prism software, which was used to determine EC50 values. See Example 4.

FIG. 3. A schematic of a simplified in vitro model of an endothelial barrier is depicted. See Example 5.

FIG. 4. Anti-Tie2 antibodies are able to reduce VEGF-induced cell permeability in an in vitro model of endothelial barrier leak. Results are represented as % of Ang1 activity (normalized to PBS treated cells). For statistical analyses (using the one-way ANOVA test), the effect of each antibody was compared to the effect of non-specific IgG1 control antibodies. Symbol meaning: **P≤0.01; ****≤0.0001. See Example 5.

FIGS. 5A to 5F. Anti-Tie2 antibodies in combination with saturating amounts of Ang2 have an additive effect on Tie2 downstream signaling in HUVEC cells. HUVEC cells were serum starved, treated with decreasing amounts of Ang2, lysed, and analyzed for the levels of pERK and total ERK. Normalized values are plotted on the Y-axis; concentrations of Ang2 are plotted on X axis (FIG. 5A). Images were quantitated using a LI-COR scanning fluorometer and pERK levels were normalized to total ERK. Serum starved HUVEC cells were treated with 43 nM Ang2 and 12 nM anti-Tie2 antibodies for 20 minutes. Lysates were submitted to western blotting and images were quantitated for pERK and ERK (FIG. 5C), and pAKT and AKT (FIG. 5E) respectively. pERK (FIG. 5D) and pAKT (FIG. 5F) levels were normalized to total ERK and Akt levels, respectively, to control for equivalent protein loading. See Example 6.

FIGS. 6A to 6B. Anti-Tie2 antibodies do not compete with Ang1 (FIG. 6A) or Ang2 (FIG. 6B) for binding to Tie2 extracellular domain (ECD). Representative competition binding data using biolayer interferometry (BLI) is depicted. The left-side panels show loading of his-tagged Tie2 ECD protein (100 nM) onto the capture biosensor (Ni-NTA) with initiation of exposure indicated by arrow (1), followed by exposure to solutions containing individual anti-Tie2 antibodies (100 nM) as indicated by arrow (2), followed by exposure to solutions containing Ang1 (FIG. 6A) or Ang2 ligand (FIG. 6B) (both at 100 nM) as indicated by arrow (3). Y-axis depicts magnitude of binding to biosensor, x-axis is time elapsed. See Example 7.

FIG. 7. Anti-Tie2 antibodies show diversity in epitope recognition by BLI binding assessment. Recombinant human Tie2 ECD protein was loaded onto each biosensor, which were subsequently exposed to a solution containing an anti-Tie2 antibody (y-axis), followed by exposure to a 2nd anti-Tie2 antibody (x-axis). Total wavelength shift (in nm) following exposure to the second antibody is annotated in each box in white numbers. Black boxes indicate lack of binding (values<0.2 nm) of the second antibody. Lack of binding was observed only with the same antibody pairs, indicating that all the antibodies tested in this panel bind to different epitopes found on the Tie2 ECD. See Example 9.

FIGS. 8A to 8B. Systemic administration of an anti-Tie2 antibody did not appear to reduce neovascularization (NV) (FIG. 8A) but reduces vaso-obliteration (VO) in an oxygen-induced retinopathy (OIR) model (FIG. 8B). Each data point represents an average of 2 eyes from single pup. Bars represent means+/−SEM; N=7-8 1-way ANOVA; Tukey's post hoc |*p<0.05; **p<0.01; ***p<0.001. See Example 10.

FIGS. 9A to 9B. Anti-Tie2 antibodies inhibit laser-induced choroidal neovascularization (CNV) in a mouse model. Quantification of neovascularization in lesion area and vascular density in retina was carried out by Image J. P values were assessed by Student's t test (significant change, p<0.05). See Example 11.

FIGS. 10A to 10B. Schematics of Tie2/VEGF bispecific constructs of the invention. FIG. 10A shows a schematic of an anti-Tie2 antibody linked via polypeptide linkers to two VEGF scFv antibody fragments. Exemplary constructs are embodied in antibody clone #57 and in antibody clone #58. FIG. 10B shows a schematic of two anti-Tie2 scFv antibody fragments linked to two VEGF-trap proteins comprised of R1-D2 and R3-D3. Exemplary constructs are embodied in antibody clone #55 and #56. See Example 13.

FIG. 11. Tie2 agonism of Tie2/VEGF bispecific constructs was assessed by western blot. Controls used were untreated and an anti-Ang1 antibody (Ang1). Positive controls were an anti-Tie2 antibody clone #3 or a non-specific antibody (NS). Test bispecific antibodies were an anti-Tie2/VEGF bispecific (antibody clone #54) or a non-specific antibody/VEGF bispecific (NS/VEGF) or a VEGF-trap/aTie2 (antibody clone #55) or a VEGF-trap alone. See Examples 3 and 14.

FIGS. 12A to 12C. Schematics of tetravalent and biparatopic anti-Tie2 antibody constructs of the invention to increase valency to Tie2 or cross-link multiple Tie2 epitopes. Exemplary tetravalent anti-Tie2 constructs having sequences of the heavy and light chains of an anti-Tie2 antibody linked via a polypeptide linker to an anti-Tie2 scFv sequence on the C-terminus of the IgG heavy chain (FIG. 12A) or the N-terminus of the heavy chain (FIG. 12B). Exemplary biparatopic anti-Tie2 antibody constructs having sequences of two anti-Tie2 scFvs from one antibody clone (aTie2 scFv B) linked via polypeptide linkers to the C-terminus of the IgG heavy chain of a second, different anti-Tie2 antibody clone (aTie2 Fab A) (FIG. 12C). In all cases, the Tie2 binding site is indicated by the hatched boxes. See Example 15.

FIG. 13. Schematic of a hexamerized anti-Tie2 antibody construct of the invention to enhance oligomerization and thereby increase agonism of Tie2. An exemplary hexamerized anti-Tie2 construct having sequences of the heavy and light chains of an anti-Tie2 antibody possessing the E430G Fc mutation is shown. See Example 15.

FIG. 14. Tie2 agonism of various anti-Tie2 antibody constructs of the invention were all assessed by a pAKT/pERK western blot. Controls used were untreated, an anti-Ang1 antibody (Ang1), anti-Tie2 antibody clone #3 (aTie2) or a non-specific antibody (NS). Test antibody constructs were a hexamerized anti-Tie2 antibody with the E430G Fc mutation (antibody clone #50) and its control, a non-specific hexamerized antibody (NS E430G), two tetravalent anti-Tie2 antibody constructs (antibody clones #51 and #52), and a divalent anti-Tie2 antibody clone #2. See Examples 3, 14 and 15.

### DETAILED DESCRIPTION OF THE INVENTION

### I. Definitions

“Tie2” is also known as angiopoietin-1 receptor, or TEK receptor tyrosine kinase, or CD202B (cluster of differentiation 202B), and is a protein that in humans is encoded by the TEK gene (Partanen Jet al., (April 1992). A novel endothelial cell surface receptor tyrosine kinase with extracellular epidermal growth factor homology domains. Molecular and Cellular Biology. 12 (4): 1698-707). This receptor possesses a unique extracellular domain containing three immunoglobulin-like loops, three epidermal growth factor-like repeats and three fibronectin type III-like repeats (see Fiedler et al., 2006. Angiopoietins: A Link between Angiogenesis and Inflammation. Trends in Immunology 27 (12): 552-58; Barton et al., Crystal structures of the Tie2 receptor ectodomain and the angiopoietin-2—Tie2 complex. Nature Struc. & Mol. Biology, 13, pp 524-532 (2006)). The contact residues for angiopoietin-1 and angiopoietin-2 are mostly overlapping on the Tie-2 receptor and are predominantly located in the second Ig-like loop, as suggested by the analysis of the crystal structure of the Ang2/Tie2 complex (Barton et al., Nat Str Biol 2006). Other work supports the concept that the binding domains for Ang1 and Ang2 are similar or identical (Fiedler et al., Angiopoietin-1 and angiopoietin-2 share the same binding domains in the Tie-2 receptor involving the first Ig-like loop and the epidermal growth factor-like repeats. JBC. Vol. 278 (3): 1721-7 (2003)). The amino acid sequence of an exemplary human Tie2 may be found under UniProt Accession Number Q02763 (SEQ ID NO:241).

The term “about” as used herein refers to the usual error range for the respective value readily known to the skilled person in this technical field. Reference to “about” a value or parameter herein includes (and describes) embodiments that are directed to that value or parameter per se.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Active” or “activity” or “biological activity” in the context of an antibody of the present invention is the ability to agonize (partially or fully activate) a biological activity of its target, for example, in vitro and/or in vivo. One example of a biological activity of an antibody is the ability to achieve a measurable improvement in the state, e.g., pathology, of a disorder associated with its target. For example, for an anti-Tie2 antibody, the disorder may be a Tie2-associated disorder, such as, for example, AMD (e.g., geographic atrophy). The activity of an anti-Tie2 antibody can be determined in in vitro or in vivo tests, including binding assays, activity assays (e.g., FRET-based activity assays (e.g., using an H2-Opt substrate) or mass spectrometry-based activity assays or signal transduction assays), using a relevant animal model, or human clinical trials. The activity of an anti-Tie2 antibody of the invention can be determined in in vitro or in vivo tests, including binding assays, alternative pathway hemolysis assays (e.g., assays measuring inhibition of the alternative pathway complement activity or activation), using a relevant animal model, or human clinical trials.

The term “active site on Tie2” is defined as the Ang 1/Ang 2 binding domain on Tie2, which is known to be within the Ig2-like domain of the extracellular domain of Tie2.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein.

An “affinity matured” antibody refers to an antibody with one or more alterations in one or more hypervariable regions (CDRs) and/or framework regions (FRs), compared to a parent antibody which does not possess such alterations, such alterations resulting in an improvement in the affinity of the antibody for antigen.

“Allosteric activation of Tie2” is the activation of Tie2 by an agonistic anti-Tie2 antibody that specifically interacts with regions of Tie2 outside of the described ligand binding or active site, such that the binding results in a change in Tie2 conformation or clustering that enhances the receptor's activity.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire light (L) chain along with the variable region domain of the heavy (H) chain (VH), and the first constant domain of one heavy chain (CH1). Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md. (1991).

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

“Single-chain Fv” also abbreviated as “sFv” or “scFv” are antibody fragments that comprise the VH and VL antibody domains connected into a single polypeptide chain. The sFv polypeptide may further comprise a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448, 1993.

A “blocking” antibody or an “antagonist” antibody is one which inhibits or reduces biological activity of the antigen it binds. Certain blocking antibodies or antagonist antibodies substantially or completely inhibit the biological activity of the antigen.

An “agonistic” or “activating” antibody is one which activates, stimulates or increases biological or signaling activity of the antigen it binds. In some situations, it is contemplated that an agonistic antibody can act in a similar manner to how a ligand engages and activates its cognate receptor. In other situations, it is contemplated that the anti-Tie2 antibodies of the invention are considered agonistic if they induce Tie2 signaling as determined by increased levels of one or more of intracellular phosphorylated Tie2 (pTie2), and/or phosphorylated Akt (pAkt), and/or phosphorylated ERK (PERK), as described in Example 3. In addition, it is further contemplated that an agonistic Tie2 antibody of the invention can also be capable of activating downstream signaling of its target antigen in the presence or absence of endogenous activating (i.e. Ang1) or inhibitory (i.e. Ang2) ligands, as described in Examples 6 and 7.

An “antibody that binds to the same epitope” as a reference antibody refers to an antibody that contacts an overlapping set of amino acid residues of the antigen as compared to the reference antibody or blocks binding of the reference antibody to its antigen in a competition assay by 50% or more. The amino acid residues of an antibody that contact an antigen can be determined, for example, by determining the crystal structure of the antibody in complex with the antigen or by performing hydrogen/deuterium exchange. In some embodiments, residues of an antibody that are within 5 angstroms of the antigen are considered to contact the antigen. In some embodiments, an antibody that binds to the same epitope as a reference antibody blocks binding of the reference antibody to its antigen in a competition assay by 50% or more, and conversely, the reference antibody blocks binding of the antibody to its antigen in a competition assay by 50% or more. An exemplary competition assay is provided herein.

The term “biparatopic” as used herein, refers to a bispecific antibody where the first antigen-binding moiety and the second antigen-binding moiety bind to different epitopes on the same antigen.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ and μ, respectively.

“Complement factors” means the various proteins and glycoproteins that make up the complement cascade which is a part of the immune system that enhances (complements) the ability of antibodies and phagocytic cells to clear microbes and damaged cells from an organism, promote inflammation, and attack the pathogen's cell membrane. It is part of the innate immune system. Complement factors contemplated herein include, for example, C1, C2, C2a, C2b, C3, C3a, C3b, C4, C4a, C4b, C5, C5a, C5b, C6, C7, C8 and C9.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation.

“Framework” or “framework region” or “FR” refers to variable domain residues other than hypervariable region (CDR) residues. The FR of a variable domain generally consists of four FR domains: FR1, FR2, FR3, and FR4.

The terms “full-length antibody,” “intact antibody,” and “whole antibody” are used herein interchangeably to refer to an antibody having a structure substantially similar to a native antibody structure or having heavy chains that contain an Fc region as defined herein.

The term “fusion polypeptide” encompasses anti-Tie2 antibodies of the invention fused to, for example, an immunoglobulin Fc region. An Fc region may comprise, for example, a CH3 domain of an immunoglobulin, which may be naturally occurring or modified in some way. Such Fc fusion polypeptides can exhibit a greater half-life in vivo than the unfused counterpart. Also, a fusion to an Fc region allows for dimerization/multimerization of the fusion polypeptide. As contemplated, the Fc region may be a naturally occurring Fc region, or may be altered to improve certain qualities, such as therapeutic qualities, circulation time, decrease aggregation problems, for example. In another embodiment, a fusion polypeptide contemplates anti-Tie2 antibody fragments fused to a ligand, such as, for example, Ang1 to enhance agonistic activity of the fusion polypeptide. In another embodiment, a fusion polypeptide contemplates an anti-Tie2 antibody fragments fused to, for example, a cytokine to elicit other desired biology.

As used herein, a “hexamerized antibody” is one in which the introduction of the E430G mutation in the Fc region facilitates the natural process of antibody hexamer formation through increased intermolecular Fc-Fc interactions upon binding to membrane-bound antigens at the cell surface (Diebolder et al., Science. 2014; de Jong et al., PLoS Biol. 2016).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup Ill as in Kabat et al., supra.

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, FR residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr Op. Struct. Biol. 2:593-596 (1992).

The term “variable” refers to the fact that certain segments of the variable domains differ extensively in sequence among antibodies. The variable or “V” domain mediates antigen binding and defines specificity of a particular antibody for its particular antigen. However, the variability is not evenly distributed across the span of the variable domains. Instead, the V regions consist of relatively invariant stretches called framework regions (FRs) of 15-30 amino acids separated by shorter regions of extreme variability called “hypervariable regions” that are each 9-12 amino acids long. The term “hypervariable region” or “CDR” when used herein refers to the amino acid residues of an antibody which are responsible for antigen-binding. The hypervariable region generally comprises amino acid residues from, for example, around about residues 24-34 (L1), 50-56 (L2) and 89-97 (L3) in the VL, and around about residues 26-35 (H1), 49-65 (H2) and 95-102 (H3) in the VH (in one embodiment, H1 is around about residues 31-35); Kabat et al., Sequences of Proteins of Immunological Interest, 5th Ed. Public Health Service, National Institutes of Health, Bethesda, Md. (1991)) and/or those residues from a “hypervariable loop” (e.g., residues 26-32 (L1), 50-52 (L2), and 91-96 (L3) in the VL, and 26-32 (H1), 53-55 (H2), and 96-101 (H3) in the VH; Chothia and Lesk, J. Mol. Biol. 196:901-917 (1987). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). Accordingly, the CDR and FR sequences generally appear in the following sequence in VH (or VL): FR1-H1 (L1)-FR2-H2 (L2)-FR3-H3 (L3)-FR4. The constant domains are not involved directly in binding an antibody to an antigen, but exhibit various effector functions, such as participation of the antibody in antibody dependent cellular cytotoxicity (ADCC).

The terms “residue numbering as in Kabat,” “Kabat amino acid residue,” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or CDR of the variable domain. For example, a heavy chain variable domain may include a single amino acid insert (residue 52a according to Kabat) after residue 52 of H2 and inserted residues (e.g. residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g, Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system. Unless otherwise indicated, CDR residues and other residues in the variable domain (e.g., FR residues) are numbered herein according to Kabat et al., supra.

An “immunoconjugate” is an antibody conjugated to one or more heterologous molecule(s) that delivers a cell-killing or cell-altering activity, including but not limited to a small molecule drug (inhibitor or activator), or cytotoxic agent, such as chemotherapeutic agents or drugs, growth inhibitory agents, toxins (e.g., protein toxins, enzymatically active toxins of bacterial, fungal, plant, or animal origin, or fragments thereof), or radioactive isotopes.

The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) approaches. For a review of methods for assessment of antibody purity, see, for example, Flatman et al., J. Chromatogr. B 848:79-87 (2007). In certain embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “multispecific antibody” is used in the broadest sense and specifically covers an antibody or antibody fragment having polyepitopic specificity (i.e., is capable of binding to two different epitopes on one biological molecule or each epitope on a different biological molecule). Such multispecific antibodies include, but are not limited to, full-length antibodies, antibodies having two or more VL and VH domains, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). “Dual specificity” or “bispecificity” refers to the ability to specifically bind to two different epitopes on the same or different target(s). However, in contrast to bispecific antibodies, dual-specific antibodies have two antigen-binding arms that are identical in amino acid sequence and each Fab arm is capable of recognizing two antigens. Dual-specificity allows the antibodies to interact with high affinity with two different antigens as a single Fab or IgG molecule. According to one embodiment, the multispecific antibody in an IgG1 form binds to each epitope with an affinity of 5 μM to 0.001 pM, 3 μM to 0.001 pM, 1 μM to 0.001 pM, 0.5 μM to 0.001 pM or 0.1 μM to 0.001 pM. “Monospecific” refers to an antibody having the ability to bind only one epitope on a particular antigen.

With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In one embodiment, the term “specific binding” refers to binding where a molecule, such as an antibody of the invention, binds to a particular polypeptide or epitope on a particular polypeptide without substantially binding to any other polypeptide or polypeptide epitope.

The term “non-ligand competitive binder” is an anti-Tie2 antibody of the invention that does not compete for the active site of Tie2 with either Ang 1 or Ang 2, while still allowing either Ang 1 or Ang 2 to bind at the active site.

A “nucleic acid encoding an antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell. In some embodiments, the nucleic acid encodes an anti-Tie2 antibody.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

A protein, including an antibody, is said to be “stable” if it essentially retains the intact conformational structure and biological activity. Various analytical techniques for measuring protein stability are available in the art and are reviewed in, e.g., Peptide and Protein Drug Delivery, 247-301, Vincent Lee Ed., Marcel Dekker, Inc., New York, N.Y., Pubs. (1991) and Jones (1993) Adv. Drug Delivery Rev. 10: 29-90. An antibody variant with “improved stability” refers to an antibody variant that is more stable comparing to the starting reference antibody. Antibody variants with improved stability are variants of the reference (wild-type) antibodies in which specific amino acid residues are altered for the purpose of improving physical stability, and/or chemical stability, and/or biological activity, and/or reducing immunogenicity of the native antibodies.

In certain embodiments, the anti-Tie2 antibody can be used as a therapeutic agent in targeting and interfering with diseases or conditions wherein the Tie2 activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062).

The term “trispecific” as used herein, refers to a type of antibody which possesses three antigen-recognition and binding sites, some of which may bind to Tie2.In another case, at least one binding arm specifically binds Tie2 and other binding sites may bind to either Tie2 or another antigen of interest (listed under “bispecific antibodies”). In one aspect, such trispecific antibodies comprise antibody fragments (e.g., Fabs, scFvs, single-domain antibodies). As a non-limiting example, three antibody-binding fragments of the invention may be assembled into a trispecific antibody such that at least one antibody-binding fragments bind Tie2 and the remaining antibody-binding fragments bind another antigen, such as, for example, VEGF. See Runcie et al., Bi-specific and Tri-specific antibodies—the next big thing in solid tumor therapeutics., Mol. Med., 24, (50)(2018).

The term “tetravalent” as used herein, refers to a type of antibody which possesses four antigen-recognition and binding sites, some of which may bind to Tie2. In another case, at least one binding arm specifically binds Tie2 and other binding sites may bind to either Tie2 or another antigen of interest (listed under “bispecific antibodies”). In one aspect, such tetravalent antibodies comprise antibody fragments (e.g., Fabs, scFvs, single-domain antibodies). As a non-limiting example, four antibody-binding fragments of the invention may be assembled into a tetravalent antibody such that two antibody-binding fragments bind Tie2 and the other two antibody-binding fragments bind another antigen, such as, for example, VEGF.

The term “pentavalent” as used herein, refers to a type of antibody which possesses five antigen-recognition and binding sites, some of which may bind to Tie2. In another case, at least one binding arm specifically binds Tie2 and other binding sites may bind to either Tie2 or another antigen of interest (listed under “bispecific antibodies”. In one aspect, such pentavalent antibodies comprise antibody fragments (e.g., Fabs, scFvs, single-domain antibodies). As a non-limiting example, five antibody-binding fragments of the invention may be assembled into a pentavalent antibody such that at least one antibody-binding fragment binds Tie2 and the other antibody-binding fragments bind another antigen, such as, for example, VEGF.

The term “hexavalent” as used herein, refers to a type of antibody which possesses six antigen-recognition and binding sites, some of which may bind to Tie2. In another case, at least one binding arm specifically binds Tie2 and other binding sites may bind to either Tie2 or another antigen of interest, listed under “bispecific antibodies”. In one aspect, such hexavalent antibodies comprise antibody fragments (e.g., Fabs, scFvs, single-domain antibodies). As a non-limiting example, six antibody-binding fragments of the invention may be assembled into a hexavalent antibody such that at least one antibody-binding fragment binds Tie2 and the other antibody-binding fragments bind another antigen, such as, for example, VEGF.

A “polypeptide linker” as used herein is a polypeptide comprising two or more amino acid residues joined by peptide bonds that are used to link two polypeptides (e.g., a VH and VL domain, two scFv antibody fragments or a variable domain and an extracellular trap protein or a scFv antibody fragment and an extracellular trap protein). Linkers may be flexible or rigid/non-flexible. Examples of such linker polypeptides are well known in the art (see, e.g., Hollinger P, et al., PNAS USA. 90:6444-6448 (1993); Poljak R J. Structure 2: 1121-1123 (1994)). Non-limiting examples of suitable, non-immunogenic linker peptides are: (G4S)n (SEQ ID NO:290), (SG4)n (SEQ ID NO:291) or G4(SG4)n (SEQ ID NO:292) flexible peptide linkers, or rigid/non-flexible linkers (EAAAK)n (SEQ ID NO:293) or (XP)n, in both cases where “n” is a number between 1 and 10, or between 1 and 4, as well as oligomers of such linkers.

A “Tie2 dysregulation disease” is any condition that would benefit from treatment with the anti-Tie2 antibody of the invention. Non-limiting examples of diseases to be treated herein include, without limitation, any disease or disorder that results from an imbalance or disruption of the Ang-1, Ang-2, Ang-3 or Ang-4 and Tie2 interactions. Non-limiting examples may encompass, for example, infectious diseases, acute respiratory distress syndrome (ARDS), ischemic injuries, ocular disorders, radiation injury, cancer, systemic sclerosis, traumatic brain injury, radiation injury, wound healing, myocardial infarction, blood brain barrier compromise (i.e. in Alzheimer's disease or other neurodegenerative diseases), neuroinflammation, cerebral cavernous malformations, Duchenne Muscular dystrophy (DMD) or Clarkson Disease. In one embodiment, the Tie2 dysregulated infectious disease comprise sepsis, dengue virus infection, tuberculosis, or influenza. In another embodiment, the Tie2 dysregulated disease may encompass ischemic injuries, such as, for example, diabetic nephropathy, acute kidney injury, chronic kidney disease, kidney or other organ transplantation, critical limb ischemia, traumatic brain injury or stroke. In another embodiment, the Tie2 dysregulated ocular disorders may include, for example, diabetic retinopathy, diabetic macular edema (DME), proliferative diabetic retinopathy (PDR) age-related macular degeneration (AMD), retinopathy of prematurity (ROP) or glaucoma.

As used herein, “administering” is meant a method of giving a dosage of a therapeutic (e.g., an anti-Tie2 antibody of the invention, a nucleic acid encoding an anti-Tie2 antibody of the invention) or a composition (e.g., a pharmaceutical composition, e.g., a pharmaceutical composition including an anti-Tie2 antibody of the invention) to a subject in need thereof. The compositions utilized in the methods described herein can be administered, for example, intravitreally (e.g., by intravitreal injection), ocularly (e.g., by ocular injection), intraocularly (e.g., by intraocular injection), sub-cutaneously or intravenously. The compositions utilized in the methods described herein can also be administered systemically or locally. The method of administration can vary depending on various factors (e.g., the compound or composition being administered, and the severity of the condition, disease, or disorder being treated).

As used herein, “co-administering” means the administration of two or more separate therapeutics (e.g., an anti-Tie2 antibody of the invention and an anti-VEGF antibody therapeutic or a recombinant VEGF fusion protein therapeutic) or compositions (e.g., an anti-Tie2 antibody pharmaceutical composition of the invention and an anti-VEGF antibody composition or a recombinant VEGF fusion protein composition) concurrently or at the same time to a subject in need thereof.

As used herein, “co-formulated” means two or more separate therapeutics (e.g., an anti-Tie2 antibody of the invention and an anti-VEGF antibody or a recombinant VEGF fusion protein) or compositions (e.g., an anti-Tie2 antibody pharmaceutical composition of the invention and an anti-VEGF antibody composition or a recombinant VEGF fusion protein composition) that are combined in a single formulation that is administered to a subject in need thereof.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. A “subject” may be a “patient.”

As used herein, “treatment” (and “treat” or “treating”) refers to clinical intervention in an attempt to alter the natural course of the individual being treated and can be performed either for prophylaxis or during the course of clinical pathology. Desirable effects of treatment include, but are not limited to, preventing recurrence of the disease or disorder, alleviation of symptoms, diminishment of any direct or indirect pathological consequences of the disease or disorder, decreasing the rate of disease progression, amelioration or palliation of the disease or disorder state, and remission or improved prognosis. In some embodiments, antibodies of the invention are used to delay development of a disease or disorder or to slow the progression of a disease or disorder.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

A “mutation” is a deletion, insertion, or substitution of a nucleotide(s) relative to a reference nucleotide sequence, such as a wild-type sequence.

A “variant” or “mutant” of a starting or reference polypeptide (e.g., a reference antibody or its variable domain(s)/CDR(s)), is a polypeptide that (1) has an amino acid sequence different from that of the starting or reference polypeptide and (2) was derived from the starting or reference polypeptide through either natural or artificial (man-made) mutagenesis. Such variants include, for example, deletions from, and/or insertions into and/or substitutions of, residues within the amino acid sequence of the polypeptide of interest, referred to herein as “amino acid residue alterations.” Thus, a variant CDR refers to a CDR comprising a variant sequence with respect to a starting or reference polypeptide sequence (such as that of a source antibody or antigen binding fragment). An amino acid residue alteration, in this context, refers to an amino acid different from the amino acid at the corresponding position in a starting or reference polypeptide sequence (such as that of a reference antibody or fragment thereof). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites.

A “VEGF extracellular trap protein” or “VEGF-Trap” as used herein is otherwise known as aflibercept (Eylea®, Regeneron-Bayer HealthCare, Tarrytown, NY, US). It consists of ligand-binding elements taken from the extracellular components of VEGF receptors 1 and 2 fused to the Fc portion of IgG1. It binds all isoforms of VEGF-A as well as VEGF-B and placental growth factor (PIGF) with a high affinity and essentially renders the VEGF-A and PIGF ligands unable to bind and activate cell receptors.

A “wild-type (WT)” or “reference” sequence or the sequence of a “wild-type” or “reference” protein/polypeptide, such as an CDR or a variable domain of a reference antibody, may be the reference sequence from which variant polypeptides are derived through the introduction of mutations. In general, the “wild-type” sequence for a given protein is the sequence that is most common in nature. Similarly, a “wild-type” gene sequence is the sequence for that gene which is most commonly found in nature. Mutations may be introduced into a “wild-type” gene (and thus the protein it encodes) either through natural processes or through man-induced means. The products of such processes are “variant” or “mutant” forms of the original “wild-type” protein or gene.

A “reference antibody,” as used herein, refers to an antibody or fragment thereof whose antigen-binding sequence serves as the template sequence upon which diversification according to the criteria described herein is performed. An antigen-binding sequence generally includes an antibody variable region, at least one CDR, including framework regions.

### Compositions and Methods

The invention provides novel antibodies that bind to Tie2, and methods of making and using the same, for example, for therapeutic and diagnostic uses. Antibodies of the invention are useful, e.g., for the diagnosis or treatment of various disorders, including Tie2 dysregulated disease, described herein.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds, Harwood Academic Publishers, 1995).

The anti-Tie2 antibodies of the invention described herein, as well as any of the antibodies for use in a method described herein, may have any of the features, singly or in combination, described herein.

In certain embodiments, an anti-Tie2 antibody provided herein has a dissociation constant (KD) of about 1 μM, about 100 nM, about 10 nM, about 1 nM, about 0.1 nM, about 0.01 nM, or about 0.001 nM (e.g., 10⁻⁸ M or less, e.g., from 10⁻⁸ M to 10⁻¹³ M, e.g., from 10⁻⁹ M to 10⁻¹³ M). For example, in some instances, an antibody provided herein binds human Tie2 (huTie2) with a KD of about 10 nM or lower. In some instances, an antibody provided herein binds huTie2 with a KD of about 5 nM or lower. In some instances, an antibody provided herein binds huTie2 with a KD of about 2 nM or lower. For example, in some instances, the antibody binds huTie2 with a KD between about 25 pM and about 2 nM (e.g., about 25 pM, about 50 pM, about 75 pM, about 100 pM, about 125 pM, about 150 pM, about 175 pM, about 200 pM, about 225 pM, about 250 pM, about 275 pM, about 300 pM, about 325 pM, about 350 pM, about 375 pM, about 400 pM, about 425 pM, about 450 pM, about 475 pM, about 500 pM, about 525 pM, about 550 pM, about 575 pM, about 600 pM, about 625 pM, about 650 pM, about 675 pM, about 700 pM, about 725 pM, about 750 pM, about 775 pM, about 800 pM, about 825 pM, about 850 pM, about 875 pM, about 900 pM, about 925 pM, about 950 pM, about 975 pM, about 1 nM, about 1.1 nM, about 1.2 nM, about 1.3 nM, about 1.4 nM, about 1.5 nM, about 1.6 nM, about 1.7 nM, about 1.8 nM, about 1.9 nM, or about 2 nM). In some instances, the antibody binds huTie2 with a KD between about 75 pM and about 600 pM (e.g., about 75 pM, about 100 pM, about 125 pM, about 150 pM, about 175 pM, about 200 pM, about 225 pM, about 250 pM, about 275 pM, about 300 pM, about 325 pM, about 350 pM, about 375 pM, about 400 pM, about 425 pM, about 450 pM, about 475 pM, about 500 pM, about 525 pM, about 550 pM, about 575 pM, about 600 pM). In some instances, the antibody binds huTie2 with a KD between about 75 pM and about 500 pM. In some instances, the antibody binds huTie2 with a KD between about 75 pM and about 400 pM. In some instances, the antibody binds huTie2 with a KD between about 75 pM and about 300 pM. In some instances, the antibody binds huTie2 with a KD between about 75 pM and about 200 pM. In some instances, the antibody binds huTie2 with a KD between about 75 pM and about 150 pM. In some instances, the antibody binds huTie2 with a KD between about 75 pM and about 125 pM. In some instances, the antibody binds huTie2 with a KD between about 75 pM and about 100 pM. In some instances, the antibody binds huTie2 with a KD of about 80 pM. In some instances, the antibody binds huTie2 with a KD of about 60 pM. In some instances, the antibody binds huTie2 with a KD of about 40 pM.

In one embodiment, KD is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER™ multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 PM [¹²⁵I]-antigen are mixed with serial dilutions of a Fab of interest (see Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20™) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, KD is measured using a BIACORE™ surface plasmon resonance (SPR) assay. For example, an assay using a BIACORE™-2000 or a BIACORE™-3000 (BIAcore, Inc., Piscataway, N.J.) is performed at 25° C. with immobilized antigen CM5 chips at about 10 response units (RU). In one embodiment, carboxymethylated dextran biosensor chips (CM5, BIAcore, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (about 0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN™-20) surfactant (PBST) at 25° C. at a flow rate of approximately 25 μl/min. Association rates (kₒₙ) and dissociation rates (k_{off}) are calculated using a simple one-to-one Langmuir binding model (BIACORE™ Evaluation Software version 3.2) by simultaneously fitting the association and dissociation sensorgrams. The equilibrium dissociation constant (KD) is calculated as the ratio k_{off}/kₒₙ. See, for example, Chen et al., J. Mol. Biol. 293:865-881 (1999). If the on-rate exceeds 10⁶ M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. KD may also be measured using a BIACORE™ SPR assay, known in the art.

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al., Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

In some instances, an anti-Tie2 antibody of the invention provided herein is a Fab.

In some instances, the Fab binds to Tie2 and may include at least one, two, three, four, five, or six CDRs selected from (a) a CDR-H1 comprising the amino acid sequences of any one of SEQ ID NOs: 1-40; (b) a CDR-H2 comprising the amino acid sequence of any one of SEQ ID Nos: 41-80; (c) a CDR-H3 comprising the amino acid sequence of any one of SEQ ID NOs: 81-120; (d) a CDR-L1 comprising the amino acid sequence of any one of SEQ ID NOs: 121-160; (e) a CDR-L2 comprising the amino acid sequence of any one of SEQ ID NOs: 161-200; and (f) a CDR-L3 comprising the amino acid sequence of any one of SEQ ID NOs: 201-240, or a combination of one or more of the above CDRs and one or more variants thereof having at least about 95% sequence identity (e.g., at least 95%, 96%, 97%, 98%, or 99% identity) to any one of SEQ ID NOS: 1-240.

In some instances, the Fab binds to Tie2 and comprises HCDRs 1-3 and LCDRs 1-3 comprising the SEQ ID Nos amino acid sequences as the following antibody clones:

| | HCDR1 | HCDR2 | HCDR3 | LCDR1 | LCDR2 | LCDR3 |
|---|---|---|---|---|---|---|
| Antibody | SEQ | SEQ | SEQ | SEQ | SEQ | SEQ |
| Clone # | ID No. | ID No. | ID No. | ID No. | ID No. | ID No. |
| 3 | 1 | 41 | 81 | 121 | 161 | 201 |
| 8 | 2 | 42 | 82 | 122 | 162 | 202 |
| 9 | 3 | 43 | 83 | 123 | 163 | 203 |
| 10 | 4 | 44 | 84 | 124 | 164 | 204 |
| 4 | 5 | 45 | 85 | 125 | 165 | 205 |
| 11 | 6 | 46 | 86 | 126 | 166 | 206 |
| 12 | 7 | 47 | 87 | 127 | 167 | 207 |
| 13 | 8 | 48 | 88 | 128 | 168 | 208 |
| 14 | 9 | 49 | 89 | 129 | 169 | 209 |
| 15 | 10 | 50 | 90 | 130 | 170 | 210 |
| 16 | 11 | 51 | 91 | 131 | 171 | 211 |
| 17 | 12 | 52 | 92 | 132 | 172 | 212 |
| 18 | 13 | 53 | 93 | 133 | 173 | 213 |
| 19 | 14 | 54 | 94 | 134 | 174 | 214 |
| 20 | 15 | 55 | 95 | 135 | 175 | 215 |
| 21 | 16 | 56 | 96 | 136 | 176 | 216 |
| 22 | 17 | 57 | 97 | 137 | 177 | 217 |
| 23 | 18 | 58 | 98 | 138 | 178 | 218 |
| 24 | 19 | 59 | 99 | 139 | 179 | 219 |
| 25 | 20 | 60 | 100 | 140 | 180 | 220 |
| 1 | 21 | 61 | 101 | 141 | 181 | 221 |
| 26 | 22 | 62 | 102 | 142 | 182 | 222 |
| 27 | 23 | 63 | 103 | 143 | 183 | 223 |
| 28 | 24 | 64 | 104 | 144 | 184 | 224 |
| 29 | 25 | 65 | 105 | 145 | 185 | 225 |
| 30 | 26 | 66 | 106 | 146 | 186 | 226 |
| 6 | 27 | 67 | 107 | 147 | 187 | 227 |
| 2 | 28 | 68 | 108 | 148 | 188 | 228 |
| 31 | 29 | 69 | 109 | 149 | 189 | 229 |
| 5 | 30 | 70 | 110 | 150 | 190 | 230 |
| 32 | 31 | 71 | 111 | 151 | 191 | 231 |
| 33 | 32 | 72 | 112 | 152 | 192 | 232 |
| 39 | 33 | 73 | 113 | 153 | 193 | 233 |
| 22 | 34 | 74 | 114 | 154 | 194 | 234 |
| 34 | 35 | 75 | 115 | 155 | 195 | 235 |
| 35 | 36 | 76 | 116 | 156 | 196 | 236 |
| 36 | 37 | 77 | 117 | 157 | 197 | 237 |
| 37 | 38 | 78 | 118 | 158 | 198 | 238 |
| 38 | 39 | 79 | 119 | 159 | 199 | 239 |
| 40 | 40 | 80 | 120 | 160 | 200 | 240 |

In some instances, the Fab binds to Tie2 and comprises: (a) a VH domain comprising an amino acid sequence having at least about 95% sequence identity (e.g., at least 95%, 96%, 97%, 98%, or 99% sequence identity) to, or has 100% the sequence of, the amino acid sequence of any one of SEQ ID NOs: 242, 244, 246, 248, 250, 252, 254, 256, 258, 260, 262, 264, 266, 268, 270, or 272; and (b) a VL domain comprising an amino acid sequence having at least about 95% sequence identity (e.g., at least 95%, 96%, 97%, 98%, or 99% sequence identity) to, or has 100% the sequence of, the amino acid sequence of any one of SEQ ID NOs: 242, 243, 245, 247, 249, 251, 253, 255, 257, 259, 261, 263, 265, 267, 269, 271, or 273.

In some instances, the Fab binds to Tie2 and comprises a VH domain and a VL domain comprising the amino acid sequences in the following combinations:

| Antibody Clone # | VH Domain SEQ ID No: | VL Domain SEQ ID No: |
|---|---|---|
| 1 | 242 | 243 |
| 2 | 244 | 245 |
| 3 | 246 | 247 |
| 7 | 248 | 249 |
| 41 | 250 | 251 |
| 42 | 252 | 253 |
| 43 | 254 | 255 |
| 44 | 256 | 257 |
| 45 | 258 | 259 |
| 46 | 260 | 261 |
| 6 | 262 | 263 |
| 47 | 264 | 265 |
| 48 | 266 | 267 |
| 5 | 268 | 269 |
| 49 | 270 | 271 |
| 31 | 272 | 273 |

In certain embodiments, an antibody provided herein is a chimeric antibody. Certain chimeric antibodies are described, for example, in U.S. Pat. No. 4,816,567; and Morrison et al., Proc. Natl. Acad. Sci. USA, 81:6851-6855 (1984). In one example, a chimeric antibody comprises a non-human variable region (e.g., a variable domain derived from a mouse, rat, hamster, rabbit, or non-human primate, such as a monkey) and a human constant domain. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which CDRs, for example, CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, for example, in Almagro and Fransson, Front. Biosci. 13:1619-1633 (2008), and are further described, for example, in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al., J. Immunol. 151:2296 (1993)); framework regions derived from the consensus sequence of human antibodies of a particular subgroup of light or heavy chain variable regions (see, e.g., Carter et al., Proc. Natl. Acad. Sci. USA, 89:4285 (1992); and Presta et al., J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In certain embodiments, an antibody provided herein is a human antibody. Human antibodies can be produced using various techniques known in the art. Human antibodies are described generally in van Dijk et al., Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. For review of methods for obtaining human antibodies from transgenic animals, see Lonberg, Nat. Biotech. 23:1117-1125(2005). See also, for example, U.S. Pat. Nos. 6,075,181 and 6,150,584 describing XENOMOUSE™ technology; U.S. Pat. No. 5,770,429 describing HUANTIBODIES™ technology; U.S. Pat. No. 7,041,870 describing K-M MOUSE™ technology, and U.S. Patent Application Publication No. US 2007/0061900, describing VELOCIMOUSE™ technology, and U.S. Pat. Nos 9,809,642 and 9,380,769, describing OmniChicken™ technology). Human variable regions from intact antibodies generated by such animals may be further modified, for example, by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222:581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227:381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

In certain embodiments, an antibody provided herein is a multispecific antibody, such as, for example, a bispecific antibody, a biparatopic antibody, a trispecific, a tetravalent antibody, a pentavalent antibody, a hexavalent antibody, to name a few examples. Multispecific antibodies are monoclonal antibodies that have binding specificities for at least two different sites. In certain embodiments, bispecific antibodies may bind to two or more different epitopes of Tie2. In certain embodiments, one of the binding specificities is for Tie2 and the other is for any other antigen, such as, for example, VEGF. Bispecific antibodies can be prepared as full-length antibodies or antibody fragments. Any of the anti-Tie2 antibodies described herein may be used to engineer a multispecific antibody.

In some instances, the multispecific anti-Tie2 antibodies of the invention are bispecific antibodies in which one arm binds Tie2 and the other arm binds VEGF. In other embodiments, such bispecific anti-Tie2 antibodies of the invention also have Fc mutations that abrogate ADCC and/or CDC as described herein. Such bispecific antibodies of the invention comprise a heavy chain and a light chain comprising the amino acid sequences in the following combinations:

| Antibody Clone # | Heavy Chain SEQ ID No: | Light Chain SEQ ID No: |
|---|---|---|
| 51 | 276 | 277 |
| 52 | 278 | 279 |
| 53 | 280 | 281 |
| 54 | 282 | 283 |
| 57 | 286 | 287 |
| 58 | 288 | 289 |

In other instances, the multispecific anti-Tie2 antibodies of the invention are fused with a VEGF extracellular trap protein. In still other instances, such multispecific anti-Tie2 antibody fusion proteins also have Fc mutations that abrogate ADCC and/or CDC as described herein. Non-limiting examples of such multispecific antibody fusion proteins comprise the following amino acid sequences:

| Antibody Clone # | SEQ ID No: |
|---|---|
| 55 | 284 |
| 56 | 285 |

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829,and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. immunol., 152:5368 (1994)); and preparing trispecific antibodies as described, for example, in Tutt et al., J. Immunol. 147:60 (1991).

Engineered antibodies with three or more functional antigen binding sites, including “Octopus antibodies,” are also included herein (see, e.g., US 2006/0025576A1).

The antibody or fragment herein also includes a “Dual Acting FAb” or “DAF” comprising an antigen binding site that binds to Tie2 as well as another, different antigen (see, e.g., US 2008/0069820).

In certain embodiments, amino acid sequence variants (e.g., antibody variants including one or more amino acid residue alterations) of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen binding.

In certain embodiments, antibody variants having one or more amino acid substitutions are provided. Sites of interest for substitutional mutagenesis include the CDRs and FRs. Conservative substitutions are contemplated, and such are well-known in the art.

Other amino acid substitutions are described below in reference to amino acid side chain classes. Amino acid substitutions may be introduced into an antibody of interest and the products screened for a desired activity, for example, retained/improved antigen binding, decreased immunogenicity, or improved or decreased ADCC or CDC. Amino acids may be grouped according to common side-chain properties:
(1) hydrophobic: Norleucine, Met, Ala, Val, Leu, lie:
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues and/or FR residues of a parent antibody (e.g., a humanized or human antibody). Generally, the resulting variant(s) selected for further study will have modifications (e.g., improvements) in certain biological properties (e.g., increased affinity, increased stability, increased expression, altered pI, and/or reduced immunogenicity) relative to the parent antibody and/or will have substantially retained certain biological properties of the parent antibody. An exemplary substitutional variant is an affinity matured antibody, which may be conveniently generated, for example, using phage display-based affinity maturation techniques such as those described herein. Briefly, one or more CDR residues are mutated and the variant antibodies displayed on phage and screened for a particular biological activity (e.g., binding affinity).

Alterations (e.g., substitutions) may be made in CDRs, for example, to improve antibody affinity. Such alterations may be made in CDR “hotspots,” i.e., residues encoded by codons that undergo mutation at high frequency during the somatic maturation process (see, e.g., Chowdhury, Methods Mol Biol. 207:179-196 (2008)), and/or residues that contact antigen, with the resulting variant VH or VL being tested for binding affinity. Affinity maturation by constructing and reselecting from secondary libraries has been described, for example, in Hoogenboom et al., in Methods in Molecular Biology 178:1-37 (O'Brien et al., ed., Human Press, Totowa, N.J., (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

In certain embodiments, substitutions, insertions, or deletions may occur within one or more FRs so long as such alterations do not substantially reduce the ability of the antibody to bind antigen. Such alterations may, for example, improve antibody affinity and/or stability (e.g., as assessed by an increased melting temperature).

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid residue alteration (e.g., a substitution) at one or more amino acid positions. In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity) but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991).

Non-limiting examples of in vitro assays to assess ADCC activity of a molecule of interest are described in U.S. Pat. No. 5,500,362 (see, e.g., Hellstrom et al., Proc. Nat'l Acad. Sci. USA 83:7059-7063 (1986) and Hellstrom et al., Proc. Nat'l Acad. Sci. USA 82:1499-1502 (1985); U.S. Pat. No. 5,821,337; and Bruggemann et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc., Mountain View, Calif.; and CYTOTOX 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, for example, in an animal model such as that disclosed in Clynes et al., Proc. Nat'l Acad. Sci. USA 95:652-656 (1998). C1q binding assays may also be carried out to confirm that the antibody is unable to bind C1q and hence lacks CDC activity. See, for example, C1q and C3c binding ELISA in WO 2006/029879 and WO 2005/100402. To assess complement activation, a CDC assay may be performed (see, e.g., Gazzano-Santoro et al., J. Immunol Methods 202:163 (1996); Cragg et al., Blood 101:1045-1052 (2003); and Cragg et al., Blood 103:2738-2743 (2004)). FcRn binding and in vivo clearance/half-life determinations can also be performed using methods known in the art (see, e.g., Petkova et al., Int'l. Immunol. 18(12):1759-1769 (2006)).

Antibodies of the invention may be engineered with reduced effector functions, such as decreased complement-dependent cytotoxicity (CDC), antibody-dependent cellular cytotoxicity (ADCC) and antibody-dependent cellular phagocytosis (ADCP) through a reduced affinity to the human FcγRIIIA and/or FcγRIIA and/or FcγRI and/or CIq binding. In some instances, such reduced effector functions are achieved by amino acid substitutions of one or more of the following Fc region residues: N297, L234, L235, D265 and P329, according to EU numbering as in Kabat. See U.S. Pat. Nos. 6,737,056, 7,332,581and WO/2012/130831. In some embodiments, the substitution mutation is one or more of N297G, N297A, L234A, L235A, D265A, and/or P329G. In some embodiments, the substitution mutation is an N297A or an N297G substitution mutation. In some embodiments, the substitution mutation includes the so-called “DANA” Fc mutant with substitution of residues D265 and N297 to alanine (U.S. Pat. No. 7,332,581). In some embodiments, the substitution mutation includes the so-called “DANG” mutations having residues substituted as D265A and N297G. In some embodiments, the substitution mutation includes the “LALA” Fc mutant with substitution of residues L234 and L235 to alanine (see Lund, J., et al., (1992) Mol. Immunol., 29, 53-59; and Tamm, A. and Schmidt, R. E. (1997) Int. Rev. Immunol., 16, 57-85). In other embodiments, the substitution mutation includes the “LALA-PG” Fc mutant with substitution of residues L234 and L235to alanine and the P329 to glycine (see Brünker, P., et al. (2016) Mol. Cancer Ther).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)).

In some embodiments, substitution mutations are made in the Fc region that result in a hexamerized antibody as described herein. In some embodiments, such a substitution mutation is E430G. In other embodiments, the E430G mutation may be combined with any of the reduced effector function mutations discussed above in order to abrogate CDC functioning and/or ADCC effector function. In one embodiment, the anti-Tie2 antibodies of the invention comprise a heavy chain and a light chain comprising the amino acid sequences harboring an E430G mutation and the L234A, L235A, P329G mutations in the following combination:

| Antibody Clone # | Heavy Chain SEQ ID No: | Light Chain SEQ ID No: |
|---|---|---|
| 50 | 174 | 175 |

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan & Winter, Nature 322:738-40 (1988); U.S. Pat. Nos. 5,648,260;5,624,821; and WO 94/29351 concerning other examples of Fc region variants.

The invention also provides immunoconjugates comprising an anti-Tie2 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

In another embodiment, an immunoconjugate comprises an antibody as described herein conjugated to a non-cytotoxic agent, such as, for example, the artemisinins, such as artusenate, or cannabinoids, or naltrexone, or aspirin, or statins, or metabolic agents, such as metformin, doxycycline and anthelmintic.

Conjugates of an antibody and cytotoxic or non-cytotoxic agents may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to, such conjugates prepared with cross-linker reagents including, but not limited to, BMPS, EMCS, GMBS, HBVS, LC-SMCC, MBS, MPBH, SBAP, SIA, STAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone) benzoate) which are commercially available (e.g., from Pierce Biotechnology, Inc., Rockford, III., U.S.A).

In one aspect, anti-Tie2 antibodies of the invention are useful for detecting the presence of Tie2 in a biological sample. The term “detecting” as used herein encompasses quantitative or qualitative detection. In certain embodiments, a biological sample comprises a cell or tissue. In certain embodiments, such tissues include normal and/or cancerous tissues that express Tie2 at higher levels relative to other tissues.

In one embodiment, an anti-Tie2 antibody for use in a method of diagnosis or detection is provided. In a further aspect, a method of detecting the presence of Tie2 in a biological sample is provided. In certain embodiments, the method comprises contacting the biological sample with an anti-Tie2 antibody as described herein under conditions permissive for binding of the anti-Tie2 antibody to Tie2 and detecting whether a complex is formed between the anti-Tie2 antibody and Tie2. Such method may be an in vitro or in vivo method. In one embodiment, an anti-Tie2 antibody is used to select subjects eligible for therapy with an anti-Tie2 antibody, e.g. where Tie2 is a biomarker for selection of patients.

Any of the antibodies (e.g., anti-Tie2 antibodies) described herein may be produced using recombinant methods and compositions, for example, as described in U.S. Pat. No. 4,816,567. In one embodiment, an isolated nucleic acid encoding an anti-Tie2 antibody described herein is provided. Such a nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such a nucleic acid are provided. In a further embodiment, a host cell comprising such a nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, for example, a Chinese Hamster Ovary (CHO) cell or lymphoid cell (e.g., YO, NSO, Sp20 cell). In one embodiment, a method of making an anti-Tie2 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-Tie2 antibody, nucleic acid encoding an antibody, for example, as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, for example, U.S. Pat. Nos. 5,648,237, 5,789,199, and 5,840,523. See also Charlton, Methods in Molecular Biology, Vol. 248 (B. K. C. Lo, ed., Humana Press, Totowa, N.J., 2003), pp. 245-254, describing expression of antibody fragments in E. coli. After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, for example, U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (T_{M4} cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, for example, Yazaki and Wu, Methods in Molecular Biology, Vol. 248 (B. K. C. Lo, ed., Humana Press, Totowa, N.J.), pp. 255-268 (2003).

The anti-Tie2 antibodies provided herein may be identified, screened for, or characterized for their physical/chemical properties and/or biological activities by various assays known in the art and described herein in the Examples and throughout the specification.

In one aspect, an antibody of the invention is tested for its antigen binding activity, e.g., by known methods such as ELISA, Western blot, surface plasmon resonance assays (e.g., BIACORE™), etc.

In one aspect, antigen binding activity (e.g., as indicated by KD) is measured using a BIACORE™ surface plasmon resonance (SPR) assay. For example, an assay using a BIACORE™-2000 or a BIACORE™-3000 (BIAcore, Inc., Piscataway, N.J.) is performed at 25° C. with immobilized antigen CM5 chips at about 10 response units (RU). In one embodiment, carboxymethylated dextran biosensor chips (CM5, BIAcore, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (about 0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN™-20) surfactant (PBST) at 25° C. at a flow rate of approximately 25 μl/min. Association rates (kₒₙ) and dissociation rates (k_{off}) are calculated using a simple one-to-one Langmuir binding model (BIACORE™ Evaluation Software version 3.2) by simultaneously fitting the association and dissociation sensorgrams. The equilibrium dissociation constant (KD) is calculated as the ratio k_{off}/kₒₙ. See, for example, Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16nm band-pass) at 25° C. of a 20 nM antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette. KD may also be measured using a BIACORE™ SPR assay.

In another aspect, competition assays may be used to identify an antibody that competes with an antibody as described herein for binding to Tie2. In certain embodiments, such a competing antibody binds to the same epitope (e.g., a linear or a conformational epitope) that is bound by an antibody as described herein. Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized Tie2 is incubated in a solution comprising a first labeled antibody that binds to Tie2 and a second unlabeled antibody that is being tested for its ability to compete with the first antibody for binding to Tie2. The second antibody may be present in a hybridoma supernatant. As a control, immobilized Tie2 is incubated in a solution comprising the first labeled antibody but not the second unlabeled antibody. After incubation under conditions permissive for binding of the first antibody to Tie2, excess unbound antibody is removed, and the amount of label associated with immobilized Tie2 is measured. If the amount of label associated with immobilized Tie2 is substantially reduced in the test sample relative to the control sample, then that indicates that the second antibody is competing with the first antibody for binding to Tie2. See Harlow and Lane (1988) Antibodies: A Laboratory Manual ch. 14 (Cold Spring Harbor Laboratory, Cold Spring Harbor, N.Y.).

In one aspect, assays are provided for identifying anti-Tie2 antibodies thereof having biological activity. See the Examples section. Biological activity may include, for example, activating, agonizing, increasing, enhancing one or more biological activities of Tie2. Antibodies having such biological activity in vivo and/or in vitro are also provided.

In certain embodiments, an antibody of the invention is tested for such biological activity. In certain embodiments, an anti-Tie2 antibody binds to Tie2 and reduces or inhibits its serine protease activity for one or more Tie2 substrates, including, for example, the H2-Opt substrate, α-casein, β-casein, or BODIPY™M FL casein substrates, known in the art, or any other suitable Tie2 substrate. In certain embodiments, an anti-Tie2 antibody inhibits Tie2 activity with an IC50 of less than 50 nM, 30 nM, 25 nM, 20 nM, 15 nM, 10 nM, 5 nM, 3 nM, 2.5 nM, 2 nM, 1 nM, 800 pM, 600 pM, 500 pM, 400 pM, 300 pM, 200 pM, 100 pM, 50 pM, or less for one or more Tie2 substrates.

In certain embodiments, labeled anti-Tie2 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like. In another embodiment of the invention, the antibody need not be labeled, and the presence thereof can be detected using a labeled antibody which binds to the antibody, such as, for example, an anti-horseradish peroxidase antibody, well-known in the art.

The antibodies of the present invention may be employed in any known assay method, such as competitive binding assays, direct and indirect sandwich assays, and immunoprecipitation assays. Zola et al., Monoclonal Antibodies: A Manual of Techniques, pp. 147-158 (CRC Press, Inc. 1987).

Competitive binding assays rely on the ability of a labeled standard to compete with the test sample analyze for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyze that are bound to the antibodies may conveniently be separated from the standard and analyze which remain unbound.

Sandwich assays involve the use of two antibodies, each capable of binding to a different immunogenic portion, or epitope, of the protein to be detected. In a sandwich assay, the test sample analyte is bound by a first antibody which is immobilized on a solid support, and thereafter a second antibody binds to the analyte, thus forming an insoluble three-part complex. See, for example, U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

For immunohistochemistry, the sample may be fresh or frozen or may be embedded in paraffin and fixed with a preservative such as formalin, for example.

### Pharmaceutical Formulations

Therapeutic formulations of the anti-Tie2 antibody or antibody fragments or variants thereof of the invention or immunoconjugates of the invention or fusion polypeptides of the invention including combinations thereof with an anti-VEGF antibody or a recombinant VEGF fusion protein as provided herein may be prepared for storage as lyophilized formulations or aqueous solutions by mixing the polypeptide having the desired degree of purity with optional “pharmaceutically-acceptable” carriers, excipients, or stabilizers typically employed in the art (all of which are termed “excipients”). For example, buffering agents, stabilizing agents, preservatives, isotonifiers, non-ionic detergents, antioxidants and other miscellaneous additives. See e.g., Remington's Pharmaceutical Sciences, 16^{th} edition, A. Osol, Ed. (1980). Such additives must be nontoxic to the recipients at the dosages and concentrations employed.

### Therapeutic Methods and Compositions

Any of the anti-Tie2 antibodies and antibody fragments of the invention or immunoconjugates of the invention or fusion polypeptides of the invention including combinations thereof with an anti-VEGF antibody or a recombinant VEGF fusion protein as provided herein may be used in therapeutic methods for treating, preventing, and/or mitigating various diseases, including any Tie2 dysregulated disease, defined herein.

### EXAMPLES

The following are examples of methods and compositions of the invention. It is understood that various other embodiments may be practiced, given the general description provided above.

### Example 1: Immunization of Transgenic Chickens to Isolate Fully Human Anti-Tie2 Antibodies

Because the extracellular domain (ECD) of the human and chicken Tie2 orthologs are only 62% identical as opposed to the 90% identity observed between murine and human Tie2 orthologs, using chickens to generate Tie2 antibodies will vastly increase the epitope coverage that can be accessed by antibodies.

Antibodies were generated by immunizing proprietary transgenic chickens, OmniChicken® (Crystal Biosciences, Inc; Emeryville, CA) with recombinantly produced and purified human Tie2 ECD. Through the deletion of endogenous immunoglobulin-encoding genes from the chicken genome and their replacement by human immunoglobulin-encoding genes, OmniChickens are “humanized” and can produce fully human antibodies (see U.S. Pat. Nos 9,809,642 and 9,380,769).

Next, a high throughput single B cell screening and cloning approach, a gel encapsulated microenvironment (GEM) technology (Crystal Bioscience, Inc, Emeryville, CA) was used to rapidly screen millions of B cells for antibodies that specifically bind to Tie2 (see U.S. Pat. Nos 8,415,173 and 8,030,095). GEM involves co-localizing single antibody secreting B lymphocytes from immunized animals inside a gel micro-droplet containing one or multiple particulate reporters (Mettler Izquierdo et al. 2016). The reporters used were polystyrene beads coated with Tie2 antigen (ECD of human or mouse Tie2 recombinant protein) and/or cells expressing human Tie2. When a Tie2 antibody producing B cell was incorporated into the GEM, binding to antigen coated beads or Tie2-expressing cells was detected using a red fluorescent secondary antibody. Through the use of single B cell cloning technology, many properly paired heavy and light chain variable domains were found. These sequences were cloned into an antibody expression vector. A total of 236 recombinant antibodies were expressed via transient transfection for confirmation of specificity and assessment of functional activity in downstream assays.

### Example 2: Primary Antibody Screening

All 236 antibodies generated as described above in Example 1 were tested for binding to recombinant human Tie2 ECD protein using ELISA methods. The majority of the antibodies screened were found to be strong binders by ELISA, many having EC50 values in the low pM range.

Cross-reactivity to mouse Tie2 by binding to recombinant mouse Tie2 ECD protein was used as a primary screen. These binding studies demonstrated that ˜90% of the anti-Tie2 antibodies generated were cross-reactive between mouse and human Tie2 proteins, making them potentially suitable for in vivo testing in established animal models.

Next, a screen for recognition and binding to human full-length Tie2 expressed in its native conformation on the plasma membrane of living cells was performed. For these experiments, human umbilical vein endothelial cells (HUVECs) were obtained from donors. Using flow cytometry, ˜70% of the antibodies that bound to human Tie2 ECD protein by ELISA also demonstrated binding to native Tie2 expressed on cells [data not shown].

### Example 3: Agonistic Antibodies Identified by a Functional Screen

All Tie2 antibodies that bound HUVEC cells as described above in Example 2 were then tested for agonistic properties using a homogeneous immune-assay (AlphaLISA™) screening platform designed to detect antibodies-induced intracellular levels of phosphorylated (activated) ERK (PERK or p42/p44) and phosphorylated (active) Akt (pAkt), both of which are known downstream signal transduction effectors of Tie2.

In this screening method, HUVEC cells were plated in 96 well plates, serum starved for 3 hours, and then treated with Ang1 (positive control), hIgG1 (non-specific human IgG1 negative control antibody) or anti-Tie2 antibodies at a concentration of 180nM. Following a 20-minute incubation, cells were lysed and lysates were analyzed for the presence of pERK and total ERK, as well as pAkt and total Akt by specific AlphaLISA assays. To be designated as “active agonistic anti-Tie2 antibody”, antibodies clones were required to induce Tie2 signaling as determined by increased levels of intracellular pAkt and pERK (normalized to total Akt and total ERK, respectively). Antibodies that were able to induce increased levels of pERK and pAkt levels were ranked based on their activity relative to the levels of pERK and pAkt induced by Ang1 treatment. Antibodies that were capable of increasing pERK and pAkt levels ≥75% of those seen following Ang1 treatment were considered comparable to Ang1 and carried forward for further testing.

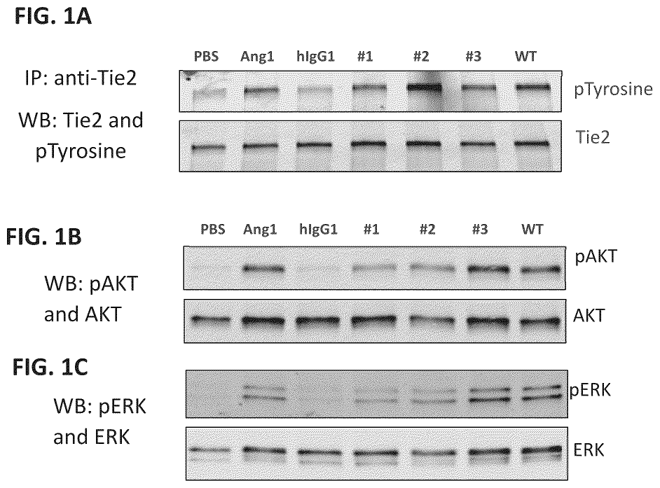

After hits were identified in the primary functional screen, the variable domains from the anti-Tie2 antibodies with the most activity were reformatted onto a different human IgG scaffold more amenable for therapeutic use. Specifically, the variable domains of all lead candidates were cloned into an expression plasmid containing a Fc domain from human IgG1 which contains an asparagine to alanine mutation at amino acid position 297 (N297A). The Tie2 agonist activity of these reformatted antibodies was confirmed in western blot analyses of antibody-induced increase in phospho-Tie2 (pTie2) (FIG. 1A) as well as by the previously described pERK and pAkt (FIGS. 1B and 1C).

Thus, anti-Tie2 antibodies of the invention were found to activate both proximal and distal Tie2 signaling events and were advanced for further studies.

### Example 4: Determining Antibody Potency In Vitro

To enable rank-ordering of anti-Tie2 antibodies of the invention based on potency, an assay with high resolution for determining antibody EC50 values was developed.

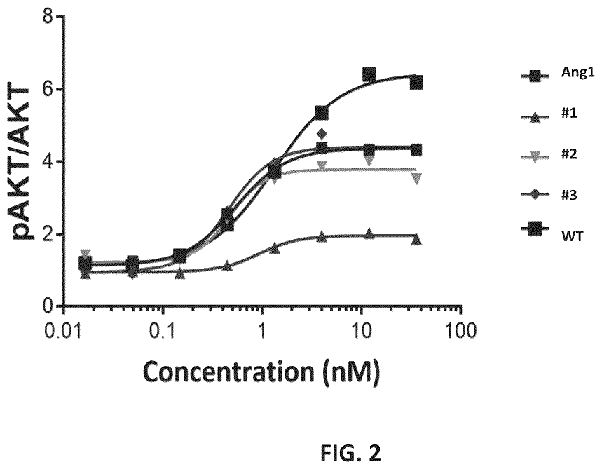

In this assay, HUVEC cells were serum starved for 3 hours, then treated with increasing concentrations of Ang1, anti-Tie2 antibodies, or negative control hIgG1 for 20 minutes prior to cell lysis. Cell lysates were then subjected to western blotting and quantitative fluorescence imaging to determine the levels of pAkt relative to total Akt and analyzed to determine respective EC50 values as follows: Ang1=0.54 nM, Ab #1=0.91 nM, Ab #2=0.48 nM, Ab #3=0.45 nM, and WT=1.33 nM. See FIG. 2.

Analysis of EC50 values revealed that most of the antibodies demonstrated potent Tie2 activation at sub-nanomolar concentrations, comparable to the potency of Ang1 in the same assay. Taken together, the functional assays utilized identified anti-Tie2 antibodies that have the ability induce Tie2 signaling to levels comparable to those seen with Ang1.

### Example 5: Anti-Tie2 Antibodies Decrease Fluid Leak in an In Vitro Model of the Endothelial Barrier

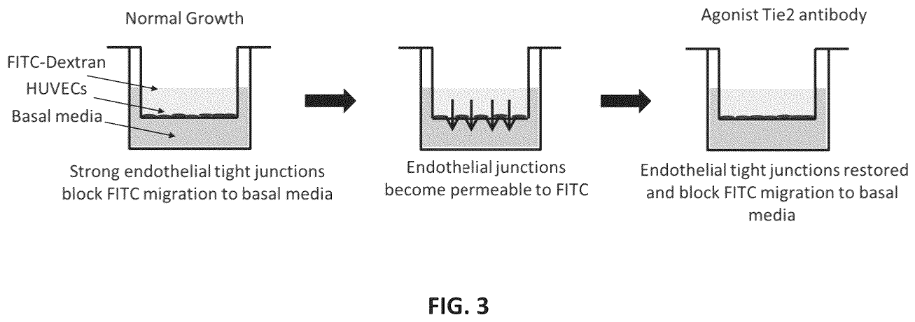

In order to investigate the potential physiological effects of anti-Tie2 antibodies on cellular ability to control permeability, a simplified in vitro model of an endothelial barrier was established. This model allowed the further characterization of the antibodies based on their ability to enhance and/or protect physiological permeability induced by increased levels of VEGF in the microenvironment. See FIG. 3 for a schematic of the model set up.

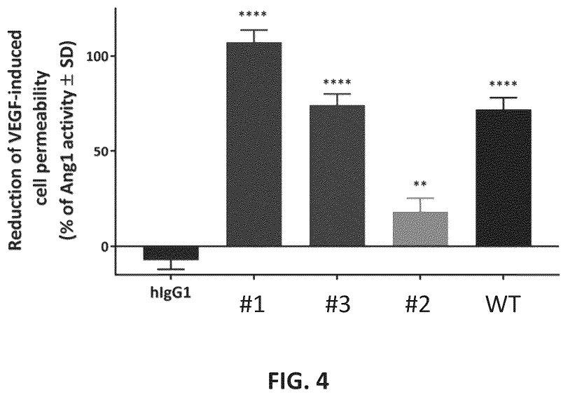

An intact confluent monolayer of HUVEC cells was cultured on a semi-permeable membrane to form an adherent structure with tight junctions. Cells were treated with 100 ng/ml of VEGF with or without Ang1 or anti-Tie2 antibodies. Permeability was assayed at different time points for 6 hours by measuring the amount of fluorescein-conjugated dextran that permeated the cell monolayer into the receiver well below the membrane. The rate of leak was measured as number of fluorescein units accumulated in the receiving well over time. The differential in endothelial barrier leak between PBS (negative control) and Ang1 (positive control) was determined. The ability of anti-Tie2 antibodies to decrease endothelial barrier leak was normalized to the effect Ang1 treatment in the same assay (% Ang1 activity). See FIG. 4.

Statistical analyses revealed that anti-Tie2 antibodies significantly reduced VEGF-induced fluid leak through this matrix of primary endothelial cells, while the human IgG1 negative control did not. Thus, anti-Tie2 antibodies were able to both stimulate downstream Tie2 signaling in biochemical assays and were also able to reduce leak in an orthogonal in vitro physiological assay.

### Example 6: Anti-Tie2 Agonist Antibodies Activate Tie2 in the Presence of High Levels of Ang2

Patients suffering from diabetic macular edema (DME) show significantly increased systemic and intravitreal levels of Ang2 (Loukovaara et al. 2013b; Regula et al. 2017). Given the potential for high levels of Ang2 to interfere with anti-Tie2 antibodies activity due to sharing a similar binding site on Tie2 or if binding of an antibody is impacted by an allosteric effect of Ang2 on Tie2 structure, the functional properties of anti-Tie2 antibodies in the presence of saturating concentrations of Ang2 were determined. Ang2 functions as a weak Tie2 agonist able to induce signaling in in vitro assays in the absence of Ang1, albeit at a lower level than can be achieved by Ang1 (Yuan et al. 2009). The in vitro experiments described below corroborated these findings.

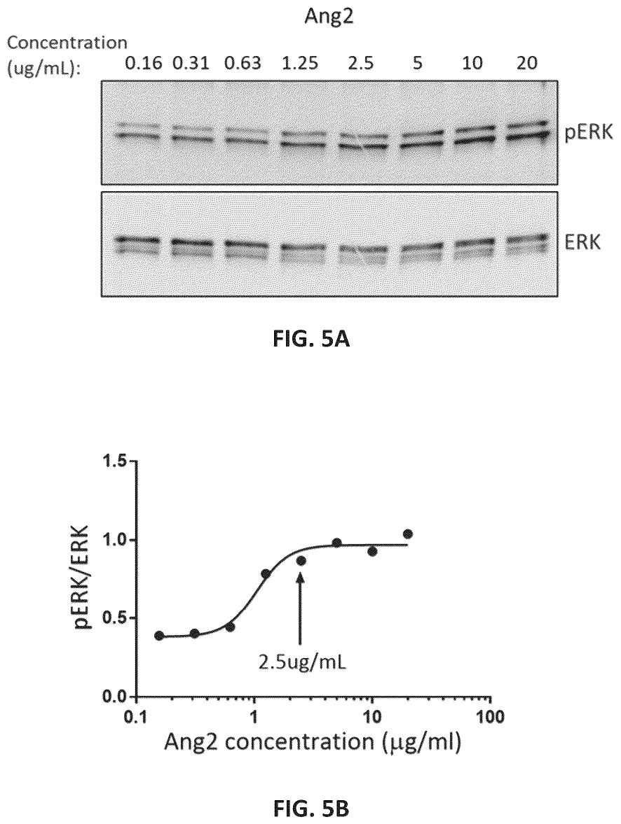

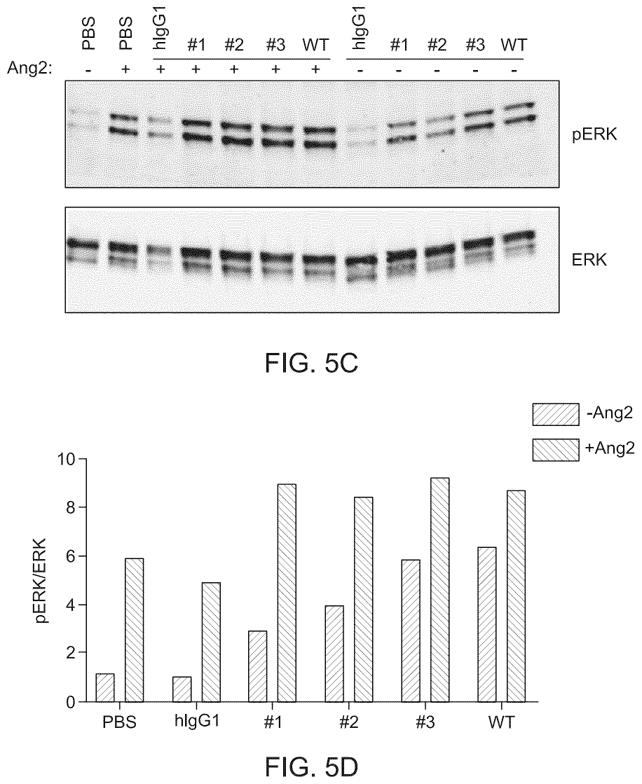

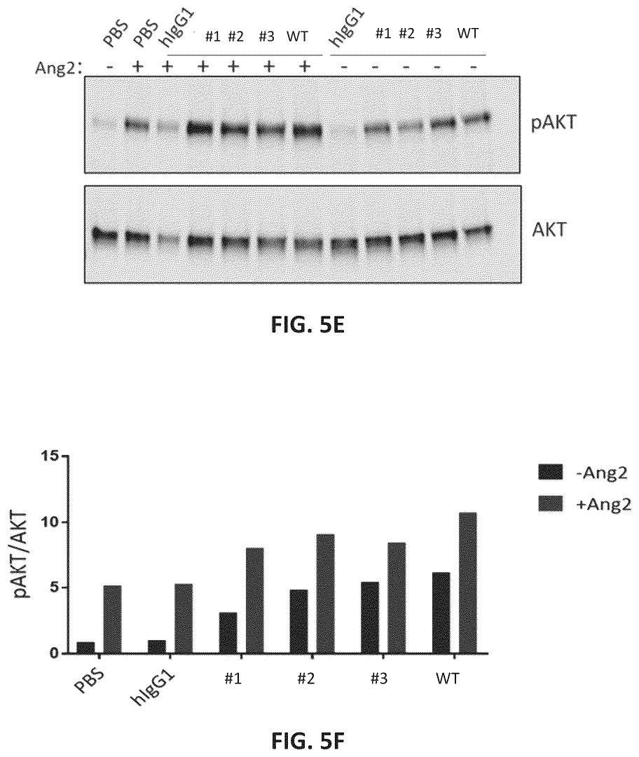

In order to determine saturating levels of Ang2 on Tie2 signaling in an in vitro model system, HUVEC cells were serum starved for 3 hours, treated with increasing concentrations of Ang2, followed by lysis and analysis of pERK/Erk levels by western blotting. See FIG. 4A. Blots were analyzed using quantitative fluorescence imaging and levels of normalized pERK were plotted as a function of Ang2 concentration. The minimum concentration of Ang2 which saturates Tie2 signaling was determined to be 2.5 ug/mL See FIG. 5B. In subsequent experiments, HUVEC cells were co-treated with Ang2 at a concentration of 2.5 ug/mL (43 nM) and anti-Tie2 antibodies at a concentration of 12 nM for 20 minutes before cell lysis and analysis of pErk/Erk (FIGS. 5C and 5D) and pAkt/Akt (FIGS. 5E and 5F) levels.

The results of these experiments demonstrated that anti-Tie2 antibodies are potent Tie2 pathway activators even in the presence of high Ang2 concentrations. Surprisingly, the Tie2 activity in cells exposed to both anti-Tie2 antibodies and Ang2 was higher than the activity seen with either treatment alone.

### Example 7: Anti Tie2 Antibodies Utilize a Non-Ligand Competitive Binding Mechanism

To further investigate the interplay between the anti-Tie2 antibodies and the angiopoietins at the level of the Tie2 receptor, a competitive binding assay was established using recombinant proteins and biolayer interferometry technology (BLI), a label-free technology for measuring biomolecular interactions.

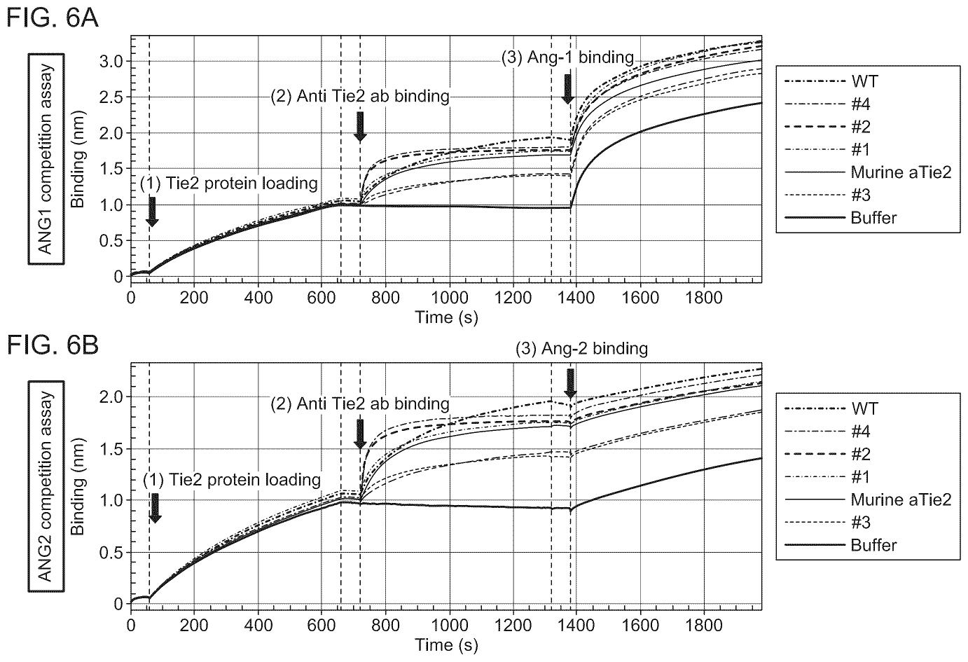

In these experiments, recombinant his-tagged Tie2 ECD was captured onto a Ni-NTA biosensor, followed by a sequential exposure to specific anti-Tie2 antibodies and then to Ang1 or Ang2 to assess potential binding competition. In these experiments, Ang1 and Ang2 were able to bind to Tie2 receptors that were pre-complexed with anti-Tie2 antibodies. See FIGS. 6A-B. Thus, the anti-Tie2 agonist antibodies of the invention were found to bind to Tie2 in a non-Ang1 and Ang2 ligand competitive manner and enhance Tie2 signaling in the presence of Ang2 to a higher level than anti-Tie2 antibody alone.

When taken together with the Tie2 pathway signaling results described above, the data characterizing the binding mechanism of the anti-Tie2 antibodies of the invention demonstrates a mode of action that is completely independent of angiopoietin levels.

### Example 8: Anti-Tie2 Agonist Antibodies are Cross-Reactive to Tie2 Variants Expressed by Other Animal Species

The cross-reactivity profile of anti-Tie2 antibodies to Tie2 orthologs expressed by preclinical animal model species was investigated, using engineered cell lines that overexpress the mouse, rat, rabbit, pig, and cynomolgus monkey Tie2 orthologs.

Briefly, expression plasmids containing the full-length Tie2 coding sequence for each species were used to transfect primary human endothelial cells (HUVECs) as well as human embryonic kidney (HEK293) cells. A stable cell population was selected by treating transfectants with puromycin antibiotic over the course of 2 weeks and single cell clones were generated using fluorescence activated cell sorting. Upon confirmation of cell surface expression of these Tie2 variants, cell lines were used to determine apparent Kd (EC50) values of anti-Tie2 antibodies on the various Tie2 proteins. To obtain this data, cross-reactivity profile of anti-Tie2 antibodies of the invention was determined. Cells were labeled with decreasing concentrations of anti-Tie2 antibodies #1, #2, and #3 (10, 5, 2.5, 1.25, 0.63, 0.31, and 0.16 ug/mL) or non-specific hIgG1 antibody (10 ug/mL), followed by labeling with a rabbit anti-human IgG antibody conjugated to an Alexa Fluor 488 fluorophore (20 ug/ml). EC50 values were generated using ForeCyt™ software. See Tables 1 and 2.

TABLE 1
Cross-Reactivity Screen (mAb at 10 ug/mL)

| | | HEK293/ | HEK293/ | HEK293/ |
|---|---|---|---|---|
| | HEK293 | Human Tie2 | Murine Tie2 | Cyno Tie2 |
| Ab #3 | 0.1 | 40.2 | 32.5 | 40.2 |
| Ab #1 | 0.2 | 60.7 | 0.4 | 74.1 |
| Ab #2 | 0.1 | 64.9 | 41.7 | 53.2 |
| Ab #5 | 0.2 | 60.2 | 0.2 | 73.1 |
| Ab #31 | 0.2 | 65.3 | 28.5 | 47.5 |
| Ab #6 | 0.1 | 63.7 | 0.3 | 78.8 |

TABLE 2

| | Apparent Kd in nM (EC50 binding) | | | | | |
|---|---|---|---|---|---|---|
| | | HEK/ | HEK/ | HEK/ | HEK/ | HEK/ |
| | HUVEC | huTie2 | mTie2 | ratTie2 | RbTie2 | cyno Tie2 |
| Ab #3 | 1.7 | 1.9 | 6.3 | 4.6 | 3.3 | 3.7 |
| Ab #1 | 2.0 | 8.0 | ND | ND | ND | 7.8 |
| Ab #2 | 1.0 | 2.7 | ND | 1.4 | 4.1 | 7.7 |

These experiments demonstrate the cross-reactive binding of the anti-Tie2 antibodies across human and preclinical species.

### Example 9: Anti-Tie2 Antibodies Recognize Distinct Epitopes on Human Tie2

In order to understand the diversity of epitope coverage of the Tie2 antibody panel, anti-Tie2 antibodies were assessed for cross-competition using BLI technology.

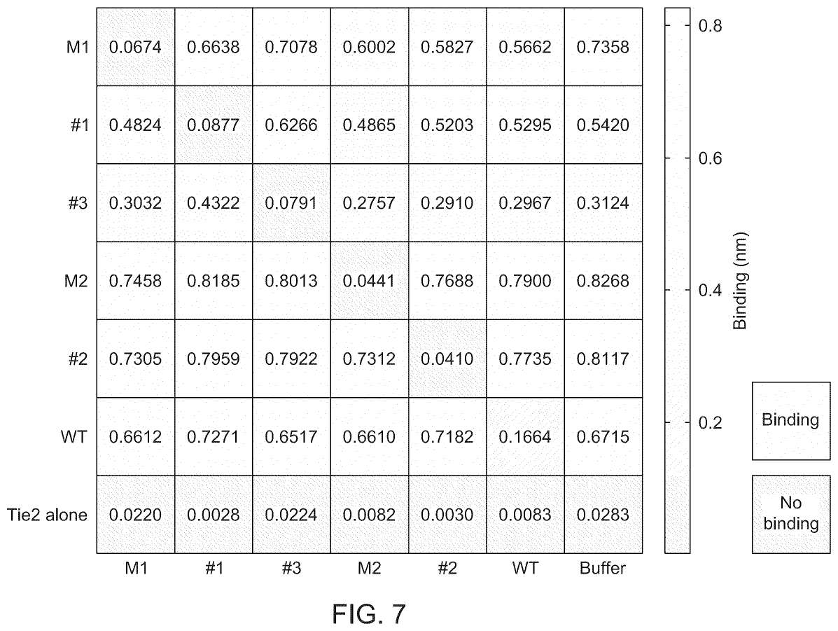

Recombinant human Tie2 ECD is loaded onto the biosensor and then exposed to anti-Tie2 antibodies (M1=murine anti-Tie2 mAb; Ab #1; Ab #3; M2=murine anti-Tie2; Ab #2; WT) to assess initial binding. The probe is then exposed to a 2nd anti-Tie2 antibody to assess the second binding event. A positive wavelength shift indicates that the second antibody can bind to the previously formed Tie2-antibody complex, indicating these antibodies bind different epitopes and do not compete for binding to Tie2 protein. Positive wavelength shifts indicating co-binding of two anti-Tie2 antibodies are indicated in grey and light grey, with shift values (nm) embedded in each cell. As expected, antibodies that were pre-bound to Tie2 prevented the binding of the same antibodies to the complex (areas shaded in black). See FIG. 7.

These experiments demonstrated that across a panel of 6 anti-Tie2 agonist antibodies, there was no cross-competition for binding to Tie2, indicating that each candidate recognizes a distinct epitope on the receptor.

### Example 10: Oxygen-Induced Retinopathy (OIR) Mouse Model

In this study, 40 C57BL/6J pups at postnatal day 7 (P7) were housed in a hyperoxic chamber (75% O₂) for 5 days (n=10 per cage) leading to vessel regression in the center of the retina. CD-1 fostering mothers were rotated before and 2-3 days after entering the chamber. At P12, pups were returned to room air where the relative hypoxia triggers abnormal neovascularization, then endotoxin free 1× PBS vehicle, 10 mg/kg; HuIgG isotype control or 10 mg/kg anti-Tie2 clone #3) were dosed intraperitoneally. At P17, all groups, including naïve OIR mice were euthanized. Eyes were enucleated and fixed in 4% paraformaldehyde for 1 hour.

Retinas were dissected and incubated overnight with rhodamine-labeled lectin from Bandeiraea simplicifolia (Griffonia simplicifolia) (1:100) in 1 mM CaCl₂ in PBS to visualize vaso-obliterated (VO) or neovascular (NV) areas. Stained retinas were flat mounted onto slides and imaged on the Zeiss® AxioScan. Images were analyzed on Visiopharm® to determine % VO or % NV of total retina.

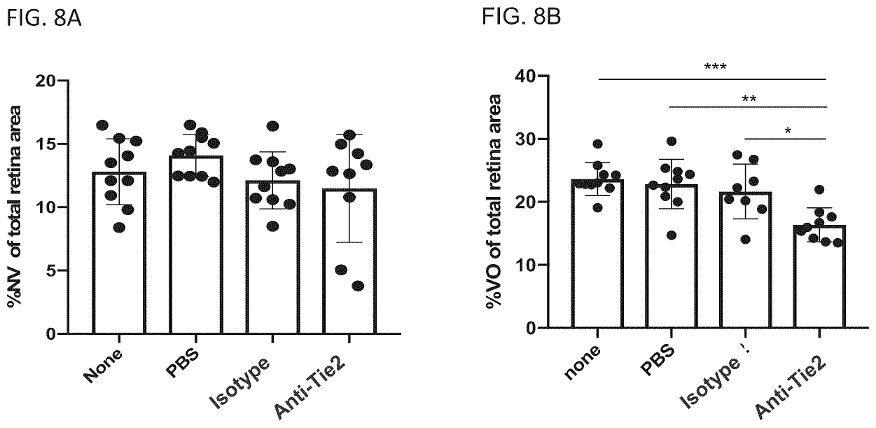

As shown in FIG. 8B, anti-Tie2 mAb, but not isotype control mAb or vehicle, was found to have a significant positive impact on the vaso-obliterated (i.e. avascular) area but did not appear to reduce NV (FIG. 8A). The promotion of the regrowth of healthy vasculature may be beneficial to mitigate the adverse consequence of chronic anti-VEGF therapies, such as geographic atrophy or capillary dropout in patients with ocular diseases. See Kim J, et al., Science Advances, Vol. 5 February 13(2019); M. Young, et al., Retina 34, 1308-1315 (2014); T. Kurihara, et al., J. Clin. Invest. 122, 4213-4217 (2012).

### Example 11: Laser-Induced Choroidal Neovascularization (CNV) Mouse Model

Male C57BL/6J mice (6-8 week) were anesthetized with a ketamine/xylazine cocktail before laser treatment. CNV lesions were induced by laser photocoagulation using a diode laser (IRIDEX®, Oculight® GL) and a slit lamp (Zeiss®) with a spot size of 50 um, power of 180 mW and exposure duration of 100 ms. Four laser burns were typically induced at 3, 6, 9 and 12 o'clock position around the optic disc in each eye. A Tie2-specific (clone #3) or isotype control antibodies or an anti-mouse VEGF control antibody (B20) were injected (10 mg/kg) intraperitoneally one day before laser induction and a total of 3 injections was performed every 3 days. Nine days after laser induction, mice were perfused with FITC-lectin or TRITC-dextran via tail vein. 5 minutes after perfusion, eyes were enucleated and fixed in 4% paraformaldehyde (PFA) for 15 min.

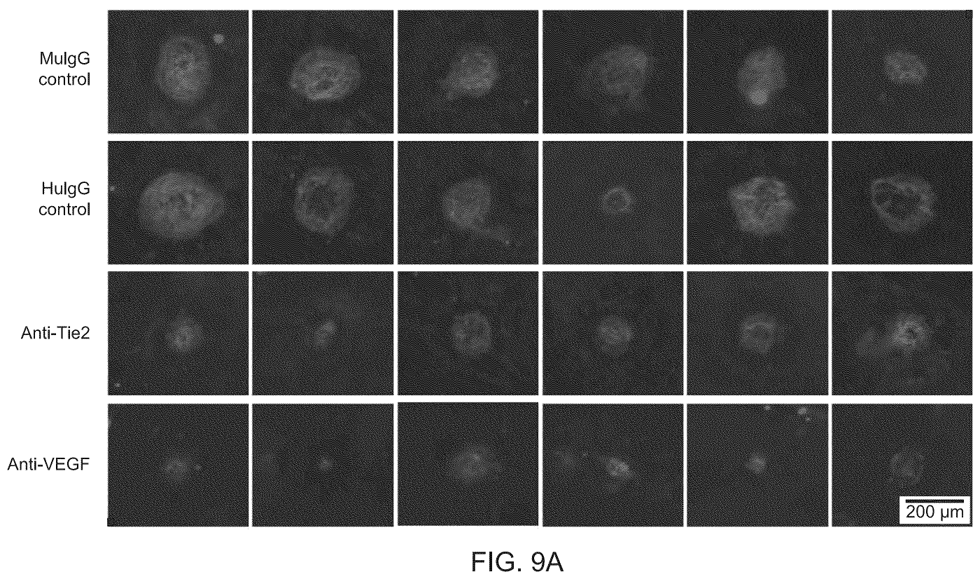

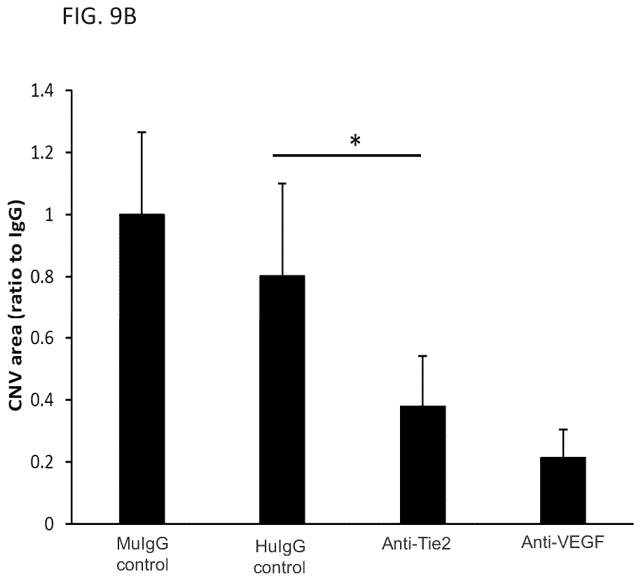

Choroid-sclera complexes and retinas were separated and anti-CD31 immunofluorescence (IF) was performed to evidence the vasculature by whole mount staining of both retina and choroidal tissues. For CD31 IF, rat anti-mouse antibody BD 550274 was diluted 1:100 and incubated overnight at 4° C. After 4-hour incubation with a secondary anti-rat antibody (Life Technologies, A11006) whole mounts were imaged at 488 nm. See FIG. 9A. Quantification of neovascularization in lesion area and vascular density in retina was carried out by Image J. P values were assessed by Student's t test (significant change, p<0.05). See FIG. 9B.

As shown in FIGS. 9A and 9B, the anti-Tie2 antibody, but not isotype control mAb, significantly inhibited choroidal neovascular lesion size following laser injury. This result indicates that the activation of Tie2 following administration of anti-Tie2 mAb has the potential to provide significant clinical benefit to humans afflicted with ophthalmological disorders such as AMD or diabetic retinopathy.

### Example 12: Streptozotocin-Induced (STZ) Diabetes Mouse Model

The administration of an agonistic Tie2 antibody of the invention may have utility for the treatment of diabetic retinopathy as well as other morbidities associated with diabetes, such as nephropathy. To explore a potential therapeutic benefit, the impact of anti-Tie2 antibodies of the invention will be tested on disease-relevant endpoints such as vascular leak in the eye, visual function as assessed by electroretinogram, production of cytokines believed to be involved with human disease, such as, for example, IL-1b, and kidney function as measured by proteinuria in a streptozotocin-induced diabetes mouse model. administered mAb via the intraperitoneal route.

C57BL/6J mice of 6- to 7-weeks in age can be weighed and their baseline glycemia measured (Accu-Chek®, Roche). Mice can be injected intraperitoneally with STZ (Sigma-Aldrich, St. Louis, MO) for 5 consecutive days at 55 mg/kg. Age-matched controls can be injected with buffer only. Glycemia can be measured again a week after the last STZ injection and mice are considered diabetic if their non-fasted glycemia is higher than 17 mM (300 mg/dL). STZ treated diabetic C57BL/6J mice can then be intravitreally (IVT) injected with appropriate amounts of an anti-Tie2 antibody of the invention, a control antibody, vehicle, or a comparator antibody, such as, for example, an anti-VEGF antibody or a recombinant VEGF fusion protein, at least 8 weeks after STZ administration.

Electroretinography (ERG) assesses global retinal cell function using the UTAS-E Visual Electrodiagnostic Test System. After overnight dark adaptation, treated mice are anesthetized with a subcutaneous injection of BW of a cocktail containing ketamine and xylazine in sterile water. One eye is proptosed and the pupil is dilated with tropicamide and phenylephrine HCL. Eyes are kept moist with Genteal® eye drops and core body temperature is maintained using a heating pad. The ERG can be recorded using ultra low impedance silver/nylon DTL Plus electrodes. Needle electrodes can be placed in the middle of the forehead and at the base of the tail. A gold contact lens electrode is used for recording ERG responses. Stimuli can consist of, for example, 50 msec flashes electronically in log steps. Responses are recorded from stimuli that range from below threshold to saturation. Analysis can include a-wave and b-wave maximum amplitudes and thresholds.

Optokinetic tracking (OKT) can also be recorded as follows. Mice can be fixed on a turntable surrounded by a collection of synchronized monitors displaying a vertical black and white grating (14°). Sinusoidal oscillation of the surrounding screens at 1 Hz and 10°/sec peak velocity is applied to induce OKT. Right eye movement is monitored with an infrared-sensitive CCD camera under illumination by ana infrared LED47. The image is sampled at 200 Hz, and the center of the pupil is calculated to estimate the eye position using Morita's Geteye software program. OKT can be recorded for 30 seconds, 3 times with intervals of about 30 seconds.

Retinal vasopermeability can be measured as followed. Mice are anesthetized and injected by tail vein with Evans blue dye dissolved in saline. Two hours after tail vein injection, mice are anesthetized with ketamine and xylazine and can be perfused through the left ventricle using saline. After perfusion, retinas are dissected, weighed and placed in formamide for 18 hours at 70° C. to extract Evans blue dye. On the next day, retinas are centrifuged for 45 minutes and removed from the formamide. Extravasation of Evans blue is measured using a plate reader at A620. A standard curve is used to convert to units of ng Evans blue/wet tissue weight.

Tissue collection for targeted transcriptome can be performed as follows. Mice are sacrificed under anesthesia, and both eyes are enucleated. Retinas are then dissected, placed into RNAlater, and processed for analysis by qRT-PCR.

### Example 13: Bispecific Biologics Constructs

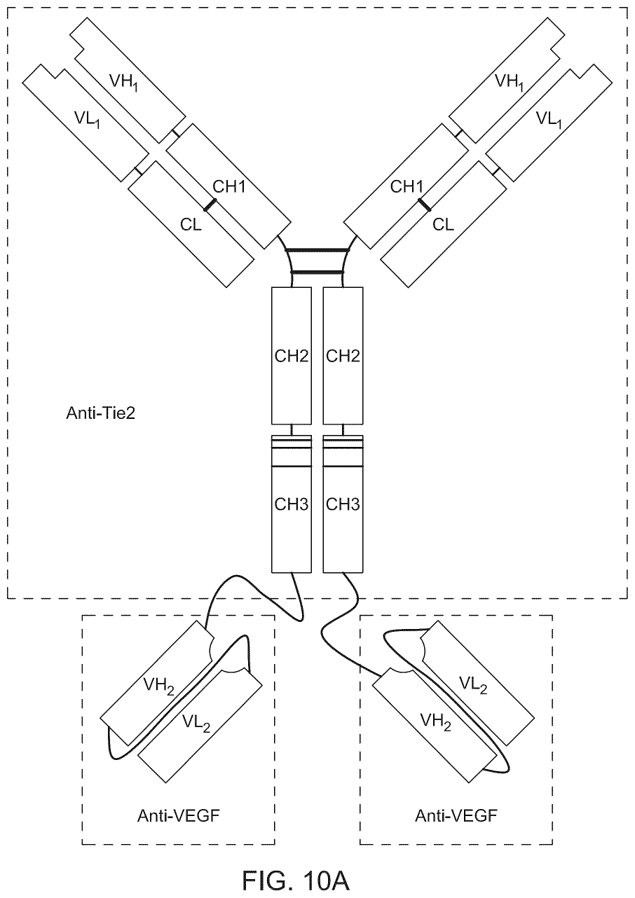

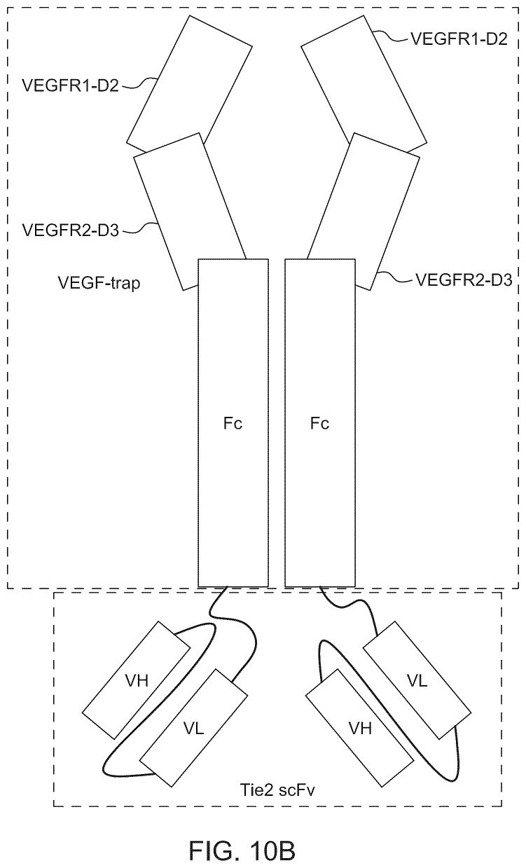

Impacting both the Tie2 and VEGFR-induced signaling pathways simultaneously may have enhanced benefit compared to modulating either pathway alone. To accomplish this, bispecific constructs designed to impact both Tie2 and VEGFR were designed, expressed and tested for activity. FIG. 10A shows an exemplary schematic of the bispecific antibodies of the invention having both Tie2 binding variable domains and VEGF binding variable domains. Antibody clone #54 was designed and generated utilizing standard cloning techniques and has the sequences of SEQ ID Nos:282 and 283. A bispecific construct comprised of the VEGFR R1D2 and R3D3 (VEGF trap proteins) and Tie2 binding domains was also generated (antibody clone #55) and has the sequence of SEQ ID No.284. Tie2 agonism was subsequently assessed for the two exemplary aTie2/VEGF bispecific constructs (antibody clone #54 and #55) using 20 nM of each in HUVEC cells using the AlphaLISA™ screening platform designed to detect antibodies-induced intracellular levels of pERK and pAkt as described in Example 3 above.

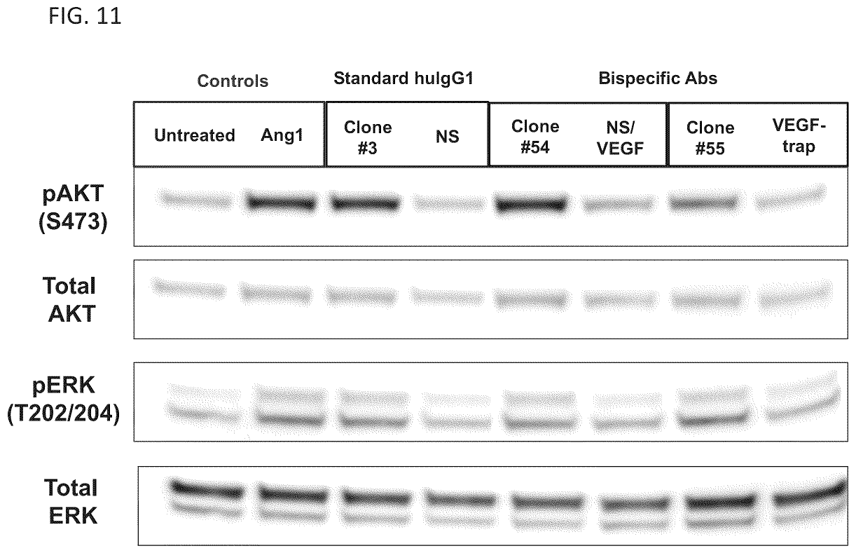

The aTie2/VEGF bispecific constructs tested were found to have the capacity to induce Tie2 signaling in vitro (FIG. 11). These bispecific molecules may allow a patient with ocular disease to receive a single drug to provide substantially more efficacy than VEGF-inhibiting or Tie2-activating monotherapies. The promotion of the regrowth of healthy vasculature provided by the Tie2 agonistic component of the bispecific molecule may also be beneficial to mitigate the potential adverse consequence of the anti-VEGF component of the molecule (i.e. geographic atrophy or capillary dropout, in patients with ocular diseases).

### Example 14: Anti-Tie2 Antibodies with Increased Valency and Biparatopic Anti-Tie2 Antibodies

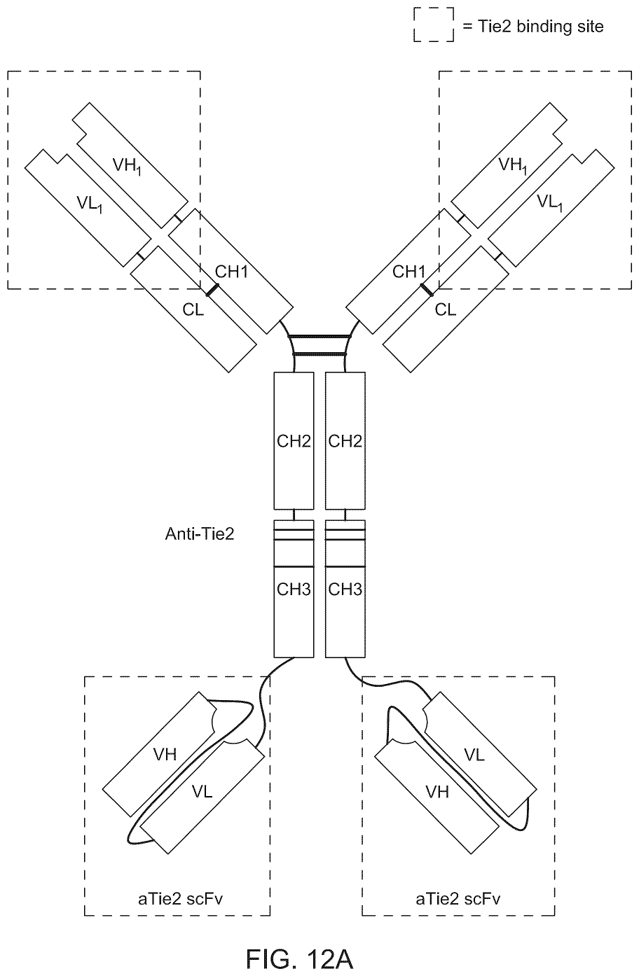

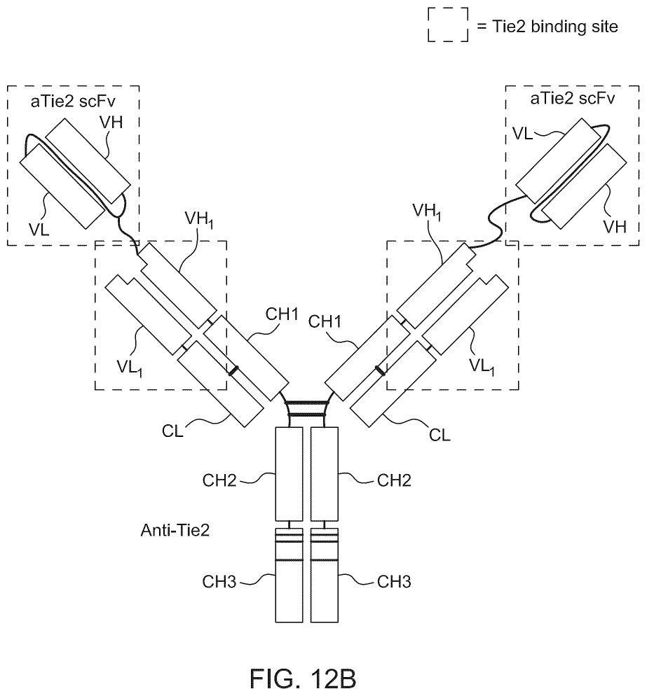

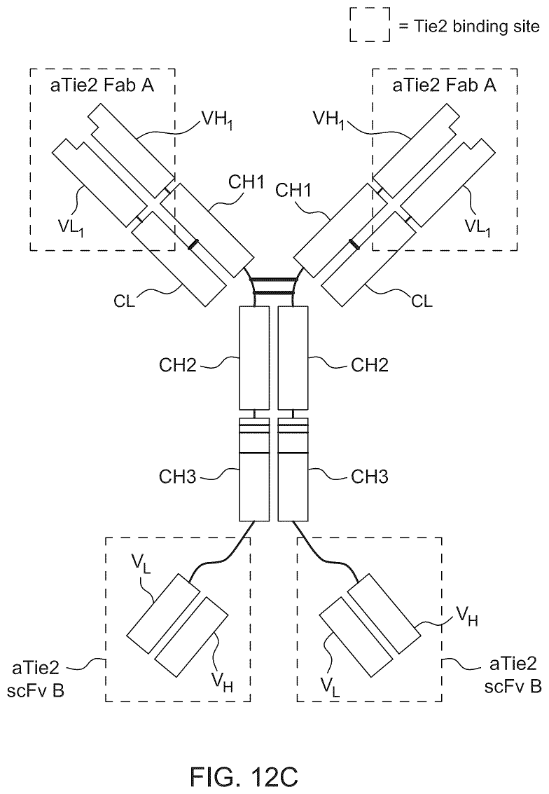

Antibodies with more than two Tie2 binding moieties, or those which may engage multiple epitopes on the Tie2 extracellular domain, may provide a means to enhance the activation of the Tie2 pathway in vivo to a greater degree than possible by divalent anti-Tie2 antibodies. An exemplary tetravalent anti-Tie2 construct having the sequences of the heavy and light chains of anti-Tie2 antibody clone #3 with the addition of a polypeptide linker and B12 scFv sequence on the C-terminus of the heavy chain (antibody clone #51) or the N-terminus of the heavy chain (antibody clone #52) were generated using standard techniques. See FIGS. 12A to 12C for exemplary schematics.

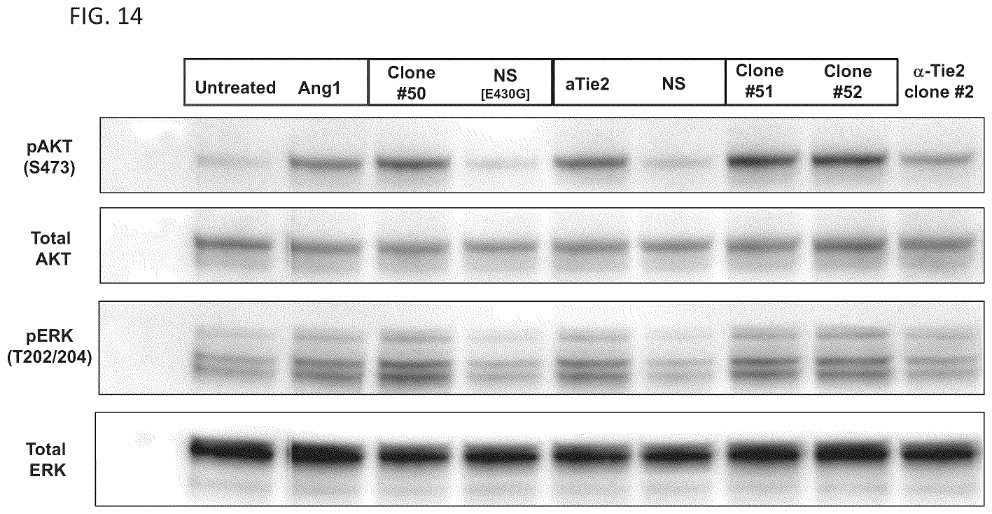

Tetravalent antibody clones #51 and #52 were assessed for their capacity to induce signal transduction through the measurement of pAkt, as described in Example 3. As shown in FIG. 14, both clones #51 and #52 demonstrate an enhanced ability to activate Tie2 signaling in HUVEC cells relative to a divalent anti-Tie2 construct (aTie2) or relative to angiopoietin 1 (Ang1). Thus, anti-Tie2 variants with additional Tie2 binding moieties may provide a means to induce higher order oligomerization of Tie2 on the cell surface and potentially provide therapeutic benefit in contexts where the levels of agonism mediated by a divalent anti-Tie2 antibody or the natural agonistic ligand Ang1 is not adequate to restore vascular homeostasis in diseased tissues.

### Example 15: Anti-Tie2 Antibodies with Fc Mutations Promoting the Ability to Hexamerize on the Cell Surface

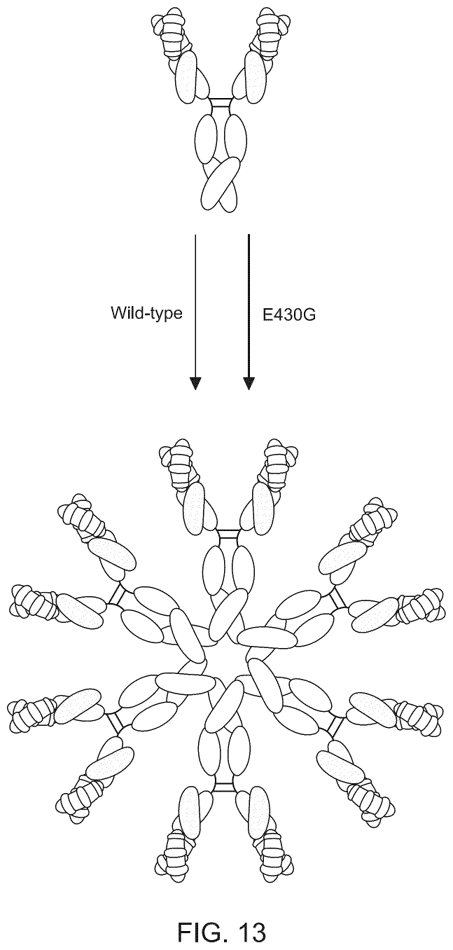

Antibodies containing specific mutations in the Fc domain of the heavy chain (i.e. E430G) have been demonstrated to enhance their capacity to form hexamers on the cell surface (M. Overdijk, Mol Cancer Ther 2020; 19:2126-38). An anti-Tie2 antibody containing an Fc domain with a E430G mutation was synthesized and compared to the same anti-Tie2 antibody with a matched Fc containing the native glutamic acid (E) at position 430. See FIG. 13 for a schematic of such a hexamerized anti-Tie2 antibody.

Tie2 agonism was assessed using the pAKT/pERK western blot as described in Example 3. As shown in FIG. 14, the anti-Tie2 E430G construct (antibody clone #50) demonstrated an enhanced ability to induce Tie2 signaling relative to the E430 construct in HUVEC cells. Thus, B12 variants with mutations increasing cell surface hexamerization potential may provide a means to induce higher order oligomerization of Tie2 on the cell surface and potentially provide therapeutic benefit in contexts where the levels of agonism mediated by an anti-Tie2 antibody or the natural agonistic ligand Ang1 is not adequate to restore vascular homeostasis in diseased tissues.

### Example 16: Determination of Anti-Tie2 Antibody Fab Binding Affinity by SPR

BIACORE™ (BIAcore, Inc., Piscataway, N.J.) was performed at 25° C. with immobilized human, rat or mouse Tie2 ECD antigens on carboxymethylated dextran biosensor chips (CM5) chips at about 10 response units (RU). CM5 chips were activated with N-ethyl-N′-(3-dimethylaminopropyl)-carbodiimide hydrochloride (EDC) and N-hydroxysuccinimide (NHS) according to the supplier's instructions. All Tie2 ECD antigens were diluted with 10 mM sodium acetate, pH 4.8, to 5 μg/ml (about 0.2 μM) before injection at a flow rate of 5 μl/minute to achieve approximately 10 RU of coupled protein. Following the injection of each Tie2 ECD, 1 M ethanolamine was injected to block unreacted groups. For kinetics measurements, two-fold serial dilutions of the antibody clone #3 Fab (0.78 nM to 500 nM) were injected in PBS with 0.05% polysorbate 20 (TWEEN™-20) surfactant (PBST) at 25° C. at a flow rate of approximately 25 μl/min. Association rates (kon) and dissociation rates (koff) were calculated using a simple one-to-one Langmuir binding model (BIACORE™ Evaluation Software version 3.2) by simultaneously fitting the association and dissociation sensorgrams. The equilibrium dissociation constant (KD) was calculated as the ratio koff/kon. See, for example, Chen et al., J. Mol. Biol. 293:865-881 (1999).

The table below shows the antibody clone #3 Fab binding affinity for each Tie2 ECD antigen tested.

| | | Ka | Kd | KD |
|---|---|---|---|---|
| Antibody | Antigen | (10⁵ M⁻¹s⁻¹) | (10⁻³s⁻¹) | (M) |
| Clone #3 | Human Tie2 ECD | 1.61E+05 | 3.77E−04 | 2.34E−09 |
| Fab | Rat Tie2 ECD | 2.54E+05 | 2.54E+05 | 2.56E−09 |
| | Mouse Tie2 ECD | 2.73E+05 | 8.76E−04 | 3.21E−09 |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1.-37. (canceled)

38. A method of treating a Tie2 dysregulated disease in a subject in need thereof, the method comprising administering to the subject an isolated anti-Tie2 antibody, or an antigen-binding fragment thereof, comprising three heavy chain complementarity-determining regions (CDR H1-3) and three light chain CDRs (CDR L1-3) as follows:

| | HCDR1 | HCDR2 | HCDR3 | LCDR1 | LCDR2 | LCDR3 |
|---|---|---|---|---|---|---|
| | SEQ | SEQ | SEQ | SEQ | SEQ | SEQ |
| | ID No. | ID No. | ID No. | ID No. | ID No. | ID No. |
| (i) | 1 | 41 | 81 | 121 | 161 | 201; |
| (ii) | 2 | 42 | 82 | 122 | 162 | 202; |
| (iii) | 3 | 43 | 83 | 123 | 163 | 203; |
| (iv) | 4 | 44 | 84 | 124 | 164 | 204; |
| (v) | 5 | 45 | 85 | 125 | 165 | 205; |
| (vi) | 6 | 46 | 86 | 126 | 166 | 206; |
| (vii) | 7 | 47 | 87 | 127 | 167 | 207; |
| (viii) | 8 | 48 | 88 | 128 | 168 | 208; |
| (ix) | 9 | 49 | 89 | 129 | 169 | 209; |
| (x) | 10 | 50 | 90 | 130 | 170 | 210; |
| (xi) | 11 | 51 | 91 | 131 | 171 | 211; |
| (xii) | 12 | 52 | 92 | 132 | 172 | 212; |
| (xiii) | 13 | 53 | 93 | 133 | 173 | 213; |
| (xiv) | 14 | 54 | 94 | 134 | 174 | 214; |
| (xv) | 15 | 55 | 95 | 135 | 175 | 215; |
| (xvi) | 16 | 56 | 96 | 136 | 176 | 216; |
| (xvii) | 17 | 57 | 97 | 137 | 177 | 217; |
| (xviii) | 18 | 58 | 98 | 138 | 178 | 218; |
| (xix) | 19 | 59 | 99 | 139 | 179 | 219; |
| (xx) | 20 | 60 | 100 | 140 | 180 | 220; |
| (xxi) | 21 | 61 | 101 | 141 | 181 | 221; |
| (xxii) | 22 | 62 | 102 | 142 | 182 | 222; |
| (xxiii) | 23 | 63 | 103 | 143 | 183 | 223; |
| (xxiv) | 24 | 64 | 104 | 144 | 184 | 224; |
| (xxv) | 25 | 65 | 105 | 145 | 185 | 225; |
| (xxvi) | 26 | 66 | 106 | 146 | 186 | 226; |
| (xxvii) | 27 | 67 | 107 | 147 | 187 | 227; |
| (xxviii) | 28 | 68 | 108 | 148 | 188 | 228; |
| (xxix) | 29 | 69 | 109 | 149 | 189 | 229; |
| (xxx) | 30 | 70 | 110 | 150 | 190 | 230; |
| (xxxi) | 31 | 71 | 111 | 151 | 191 | 231; |
| (xxxii) | 32 | 72 | 112 | 152 | 192 | 232; |
| (xxxiii) | 33 | 73 | 113 | 153 | 193 | 233; |
| (xxxiv) | 34 | 74 | 114 | 154 | 194 | 234; |
| (xxxv) | 35 | 75 | 115 | 155 | 195 | 235; |
| (xxxvi) | 36 | 76 | 116 | 156 | 196 | 236; |
| (xxxvii) | 37 | 77 | 117 | 157 | 197 | 237; |
| (xxxviii) | 38 | 78 | 118 | 158 | 198 | 238; |
| (xxxix) | 39 | 79 | 119 | 159 | 199 | 239; or |
| (xxxx) | 40 | 80 | 120 | 160 | 200 | 240. |

39. (canceled)

40. The method of claim 38, further comprising co-administering to the subject a pharmaceutical composition comprising an anti-VEGF antibody or a VEGF extracellular trap protein.

41. The method of claim 38, wherein the Tie2 dysregulated disease comprise infectious diseases, acute respiratory distress syndrome (ARDS), ischemic injuries, ocular disorders, radiation injury, cancer, systemic sclerosis, traumatic brain injury, neuroinflammation, radiation injury, wound healing, myocardial infarction, blood brain barrier compromise, cerebral cavernous malformations, Duchenne Muscular dystrophy (DMD) or Clarkson Disease.

42. The method of claim 38, wherein the Tie2 dysregulated infectious diseases comprise sepsis, dengue virus infection, tuberculosis, or influenza.

43. The method of claim 38, wherein the Tie2 dysregulated ischemic injuries comprise diabetic nephropathy, acute kidney injury, chronic kidney disease, organ transplant, critical limb ischemia, traumatic brain injury or stroke.

44. The method of claim 38, wherein the Tie2 dysregulated ocular disorders comprise diabetic retinopathy, diabetic macular edema (DME), proliferative diabetic retinopathy (PDR) age-related macular degeneration (AMD), retinopathy of prematurity (ROP), or glaucoma.

45.-46. (canceled)

47. A method of treating a Tie2 dysregulated disease in a subject in need thereof, the method comprising administering to the subject an isolated anti-Tie2 antibody, or an antigen-binding fragment thereof comprising:
a heavy chain variable (VH) domain comprising an amino acid sequence of SEQ ID NO.242 and a light chain variable (VL) domain comprising an amino acid sequence of SEQ ID NO.243;
a heavy chain variable (VH) domain comprising an amino acid sequence of SEQ ID NO.244 and a light chain variable (VL) domain comprising an amino acid sequence of SEQ ID NO.245;
a heavy chain variable (VH) domain comprising an amino acid sequence of SEQ ID NO.246 and a light chain variable (VL) domain comprising an amino acid sequence of SEQ ID NO.247;
a heavy chain variable (VH) domain comprising an amino acid sequence of SEQ ID NO.248 and a light chain variable (VL) domain comprising an amino acid sequence of SEQ ID NO.249;
a heavy chain variable (VH) domain comprising an amino acid sequence of SEQ ID NO.250 and a light chain variable (VL) domain comprising an amino acid sequence of SEQ ID NO.251;
a heavy chain variable (VH) domain comprising an amino acid sequence of SEQ ID NO.252 and a light chain variable (VL) domain comprising an amino acid sequence of SEQ ID NO.253;
a heavy chain variable (VH) domain comprising an amino acid sequence of SEQ ID NO.254 and a light chain variable (VL) domain comprising an amino acid sequence of SEQ ID NO.255;
a heavy chain variable (VH) domain comprising an amino acid sequence of SEQ ID NO.256 and a light chain variable (VL) domain comprising an amino acid sequence of SEQ ID NO.257;
a heavy chain variable (VH) domain comprising an amino acid sequence of SEQ ID NO.258 and a light chain variable (VL) domain comprising an amino acid sequence of SEQ ID NO.259;
a heavy chain variable (VH) domain comprising an amino acid sequence of SEQ ID NO.260 and a light chain variable (VL) domain comprising an amino acid sequence of SEQ ID NO.261;
a heavy chain variable (VH) domain comprising an amino acid sequence of SEQ ID NO.262 and a light chain variable (VL) domain comprising an amino acid sequence of SEQ ID NO.263;
a heavy chain variable (VH) domain comprising an amino acid sequence of SEQ ID NO.264 and a light chain variable (VL) domain comprising an amino acid sequence of SEQ ID NO.265;
a heavy chain variable (VH) domain comprising an amino acid sequence of SEQ ID NO.266 and a light chain variable (VL) domain comprising an amino acid sequence of SEQ ID NO.267;
a heavy chain variable (VH) domain comprising an amino acid sequence of SEQ ID NO.268 and a light chain variable (VL) domain comprising an amino acid sequence of SEQ ID NO.269; or
a heavy chain variable (VH) domain comprising an amino acid sequence of SEQ ID NO.270 and a light chain variable (VL) domain comprising an amino acid sequence of SEQ ID NO.271.

48. The method of claim 38, wherein the antibody is an allosteric activator of Tie2.

49. The method of claim 38, wherein the antibody is a non-ligand competitive binder of Tie2.

50. The method of claim 38, wherein the antibody is cross-reactive against human, mouse, rat, rabbit and monkey Tie2.

51. The method of claim 38, wherein said antibody is fully human, humanized, monoclonal, or chimeric.

52. The method of claim 38, wherein said antibody is monospecific.

53. The method of claim 38, wherein said antibody is multispecific.

54. The method of claim 53, wherein the multispecific antibody is bispecific.

55. The method of claim 54, wherein the bispecific antibody comprises one binding arm that specifically binds human Tie-2 of claim 8 and a second binding arm that specifically binds VEGF-A, VEGF-B, VEGF-C, VEGF variants, Ang-1, Ang-2, Ang-3, Ang-4, PDGF-β, interleukin-1β, VE-PTP, complement factor C3, integrin α5β1, amyloid beta, PD-1, PD-L1, or CTLA-4.

56. The method of claim 53, wherein the multispecific antibody is a biparatopic antibody.

57. The method of claim 56, wherein the biparatopic antibody comprises one binding arm that specifically binds a first epitope on the ECD of human Tie2 and the other binding arm that specifically binds to a second epitope on the ECD of human Tie2.

58. The method of claim 53, wherein the multispecific antibody is a trivalent, a tetravalent, a pentavalent, a hexavalent antibody, wherein the trivalent, tetravalent, pentavalent, or hexavalent antibody comprises at least one binding arm that specifically binds human Tie2 of claim 8 and other remaining binding arms that specifically binds VEGF-A, VEGF-B, VEGF-C, VEGF variants, Ang-1, Ang-2, Ang-3, Ang-4, PDGF-β, interleukin-1β, VE-PTP, complement factor C3, integrin α5β1, amyloid beta, PD-1, PD-L1, or CTLA-4.

59. The method of claim 38, wherein said antibody is an antibody fragment that specifically binds human Tie2.

60. The method of claim 59, wherein the antibody fragment is a Fab, a Fab′-SH, a Fv, a scFv, or a (Fab′)₂ fragment.

61. The method of claim 53, wherein the multispecific antibody is comprised of scFv antibody fragments linked together by a polypeptide linker.

62. The method of claim 38, wherein the antibody possesses reduced effector function.

63. The method of claim 62, wherein the antibody comprises at least one substitution mutation at amino acid residue N297, L234, L235, P329, D265, and E430 according to EU numbering as in Kabat.

64. The method of claim 63, wherein the at least one substitution mutation is selected from the group consisting of amino acid residue N297G, N297A, L234A, L235A, P329G, D265A, and E430G according to EU numbering as in Kabat.

65. The method of claim 64, wherein the antibody comprises the substitution mutation at residue N297A or N297G.

66. The method of claim 64, wherein the antibody comprises the substitution mutation at residues L234A, L235A and P329G.

67. The method of claim 64, wherein the antibody comprises the substitution mutation at residues D265A and N297G.

68. The method of claim 65, wherein the antibody further comprises the substitution mutation at residue E430G.

69. The method of claim 66, wherein the antibody further comprises the substitution mutation at residue E430G.

70. The method of claim 67, wherein the antibody further comprises the substitution mutation at residue E430G.

71. The antibody of claim 69, wherein the antibody comprises a heavy chain comprising an amino acid sequence of SEQ ID NO:174 and a light chain comprising an amino acid sequence of SEQ ID NO:175.

72. The method of claim 66, wherein the antibody comprises:
a heavy chain comprising an amino acid sequence of SEQ ID NO:276 and a light chain comprising an amino acid sequence of SEQ ID NO:277;
a heavy chain comprising an amino acid sequence of SEQ ID NO:278 and a light chain comprising an amino acid sequence of SEQ ID NO:279;
a heavy chain comprising an amino acid sequence of SEQ ID NO:280 and a light chain comprising an amino acid sequence of SEQ ID NO:281;
a heavy chain comprising an amino acid sequence of SEQ ID NO:282 and a light chain comprising an amino acid sequence of SEQ ID NO:283;
a heavy chain comprising an amino acid sequence of SEQ ID NO:286 and a light chain comprising an amino acid sequence of SEQ ID NO:287; or
a heavy chain comprising an amino acid sequence of SEQ ID NO:288 and a light chain comprising an amino acid sequence of SEQ ID NO:289.

73. The method of claim 54, wherein the antibody comprises:
an amino acid sequence of SEQ ID NO:284; or
an amino acid sequence of SEQ ID NO:285.
